SEQUENCE LISTING (35 sequences)

SEQ ID NO: 1 - protein, 619 aa, Pyrococcus abyssi
MDELVKALERAGYLLTPSAYYLLVDHFKEGKFSLVELVKFAKSKGVFIIDGDLAYEFLQFLGLGVPQEIKESYISTGEEAEKTVESQETRASELEEGGVSQVSSGELQELKEESPEISTTEEEIGGLELVQSSISTGSEVEYNNGENGESVVVLDKYGYPILYAPEEIGEEKEYSKYEDVVIEWNPSVTPVQIEKNYEVKFDVRQVKLRPPKVKNGSGKEGEIIVEAYASLFKSRLSKLKRILRENPEISNVVDIGKLNYVSGDEEVTIIGLVNSKRETNRGLIFEVEDKTGIVKVFLPKDSEDYREAFKVLPDAVVAFKGFYSKKGIFFANKFYLPDVPLYRKQKPPLEEKVYAILISDIHVGSREFCEKAFLKFLEWLNGHVESKEEEEIVSRVKYLIIAGDVVDGIGIYPGQYSDLVIPDIFDQYEALANLLANVPEHITMFIGPGNHDAARPAIPQPEFYKEYAKPIYKLKNAIIISNPAVIRLHGRDFLIAHGRGIEDVVSFVPGLTHHKPGLPMVELLKMRHLAPTFGGKVPIAPDPEDLLVIEEVPDLVQMGHVHVYDAVVYRGVQLVNSATWQAQTEFQKMVNIVPTPAKVPVVDVESARVVKVLDFSGWC

SEQ ID NO: 2 - protein, 1270 aa, Pyrococcus abyssi
MELPKEMEEYFEMLQREIDKAYEIAKKARAQGKDPSLDVEIPQATDMAGRVESLVGPPGVAKRIRELVKEYGKEIAALKIVDEIIEGKFGDLGSREKYAEQAVRTALAILTEGIVSAPIEGIANVKIKRNTWADNSEYLALYYAGPIRSSGGTAQALSVLVGDYVRRKLGLDRFKPSEKHIERMVEEVDLYHRAVTRLQYHPSPEEVRLAMRNIPIEITGEATDDVEVSHRDVPGVETNQLRGGAILVLAEGVLQKAKKLVKYIDKMGIEGWEWLKEFVEAKEKGEPKEEGKEESLAESTLEETKVEVDMGFYYSLYQKFKEEIAPSDKYAKEVIGGRPLFSDPSKPGGFRLRYGRSRASGFATWGINPATMILVDEFLAIGTQLKTERPGKGAVVTPVTTIEGPIVKLKDGSVLRVDDYNLALKVREDVEEILYLGDAVIAFGDFVENNQTLLPANYCEEWWILEFVKALKEIYEVHLEPFTENEEESIEEASDYLEIDPEFLKEMLRDPLRVKPPVELAIHFSEVLGIPLHPYYTLYWNSVEPKDVEKLWRLLKNYAEIEWSNFRGIKFAKKIVISQEKLGDSKRTLELLGLPHTVRDGNVIVDYPWAAALLTPLGNLNWEFMAKPLYATIDIINENNEIKLRDRGISWIGARMGRPEKAKERKMKPPVQVLFPIGLAGGSSRDIKKAAEEGKVAEVEIAFFKCPKCGHVGPEHLCPNCGTRKELLWVCPRCNAEYPESQAEGYNYTCPKCNVKLRPYAKRKIRPSELLNRAMENVKVYGVDKLKGVMGMTSGWKMPEPLEKGLLRAKNDVYVFKDGTIRFDATDAPITHFRPREIGVSVEKLRELGYTHDFEGKPLVSEDQIVELKPQDIILSKEAGRYLLKVAKFVDDLLEKFYGLPRFYNAEKMEDLIGHLVIGLAPHTSAGIVGRIIGFVDALVGYAHPYFHAAKRRNCDGDEDAVMLLLDALLNFSRYYLPEKRGGKMDAPLVITTRLDPREVDSEVHNMDIVRYYPLEFYEATYELKSPKELVGVIERVEDRLGKPEMYYGLKFTHDTDDIALGPKMSLYKQLGDMEEKVRRQLEVAKRIRAVDEHGVAEKILNSHLIPDLRGNLRSFTRQEFRCVKCNTKFRRPPLNGKCPVCGGKIVLTVSKGAIEKYLGTAKMLVTEYNVKNYTRQRICLTERDIDSLFENVFPETQLTLIVNPNDICQRLVMARTGEVNKSGLLENLSNGSKKTEKAEKAEKPRKKSDEKPKKKRVISLEEFFSRKSK

SEQ ID NO: 3 - protein, 725 aa, Thermococcus nautili
MLIEDLIKNKYLITPSAYYLLEPHYKRDFTLAELIKFAKARGTFVIDSSIAEAFLAEKGLFSTGELTEQTPLEVSEEEPLEASPEEIHGDFAESAEIPVSETVTASRPEEIPSSEDFESAEASGSISTGDVVESEVATASSGEESVEPAEIVGETSISTGTTLELEQSPASIPVEEPISSEISEGESFVSTGTPEHEELVDEAAGSLDSSLTEVVEDSLPVEAEPLNGNGYGNYADSEEYYENGENGNGVKPKIVYGDYGIPIAYVGEDVEGEKSYSTYEDVVIKPKEGFHYRAKEIPDEWELTFDVKNVKFEAPKVKNAASKEGEIIIQAYSSYFKSRLRKMRRIFRENPEIGTVIDIAKLSYVRDDEVTIIGLVNDKRETAKGYMFEVEDATGRVKVFLGRDKENASQAYNAIMPDSVVAFRGQPGRGIFFANRVFLPDVPKFRREKPPLEEKVYAILLSDIHVGSNKFCEKAFEKFLEWLNGEVNSRAEEELVSRIKYLIIGGDVVDGVGIYPGQYNELAIPDIFDQYEALANLLRNVPEHITMFIGPGNHDAARTALPQPGFYEEYAKPIYKLKNAVILSNPAVINLHGREFLIAHGRGIEDVVDFIPGRTHHKPAEAMVDLLKLRHLAPTFGNKVPIAPDPEDTLVIESVPDLFQAGHVHVMEYRIYNGVFVINSGTWQAQTEFQKMVNIIPTPARVPIIDVETARLRAVVSFDQFCEGV

SEQ ID NO: 4 - protein, 735 aa, Thermococcus kodakarensis
MLVEDLLKNNYLITPSAYYLLSDHYKKAFTLAELIKFAKNRGTFVVDSNLAREFLAEKGIISSGNGALDGSVLSGISVSESPVKPNGEAVLESESDESGAIESSLQEGVEDGVESIAEPSAASVEDVVVAGESSLASEGSSLEDEINTEPELSSSMDDGAEVVQSSEGDETSISTGNALGSVDYGGSDSESEALEEASSGGESFISTGTPEDEALLEESPEGEFPDENGFSDESLPVENGSENGYGDSEEYYENGDNGVKPKIVYGDYGVPIAYVADETPEEEKAYSTYSDLVIAPKEGFHYRAKEIPDEWELAFDVKNVKFEVPKVKNAQSKEGEVIIQAYSSYFKSRLKKMRRIFRENPEIGTIVDIAKLSYVREDDVTIIGLVNEKRETRKGYLFEIEDATGRIKVFIGSDKEGANEAYSTIMPDSVVAFRGTPGKGIFFANRVFLPDVPKFKRSKPPLEEKVYAILLSDIHVGSNKFCEEAFIKFLEWLNGEVNSRTEEELVSRIKYIIIGGDVVDGVGIYPGQYNELAIPDIFDQYEALANLLKQVPDHITMFIGPGNHDAARTALPQPGFYEEYAKPLYKLKNAVIISNPAVIRLHGRDFLVAHGRGIEDVVDFVPNRSHHRPAEAMVELLKLRHIAPTFGNKVPIAPDPEDTLVIESVPDLFQAGHVHVMQYKTYNGVFVINTGTWQAQTEFQKMVNIIPTPARVPIIDVETARLRAVVRFDQFCEGV

SEQ ID NO: 5 - protein, 644 aa, Palaeococcus ferrophilus
MKLVEDLIKNNYLITPPAYYTLAHHYENGSFTLAELVKFAKSRGTFVIDEGTAEEFIKWKGLISSGANGEHESVEEPSVEGMESGTEGAVPQEKELLMGESAGVENSSIEVTAVEESPAVESAPESPEFSNEDVQMEEPLTNGDETLVSNGTWAGSNGDYNGDEGAVKAVYSDYGVLVPLEEEPPEEIGAKSYSVYADFKVTPREGFRPRAAEMPSEARVEFDVRNVKLTPPKVKNAASKEGEIIIRSYELHFRSRLRKLRRILRDTNELTGVVDIGRLSYTAGYEDPVTIIGLVNSKRETRKGFMFELEDATGTVKVFIKRDNPDYSKVWEVLLDSVVAFSGYYSKRGLFFAEHIYLPDVPLYRREKPPLEEPVYAALISDIHVGSDTFCEKAFMKFMKWLNGYTASREEEEILDRLKYLIIAGDVVDGVGIYPGQYNELLIPDIFDQYEALANLLSNVPEHIQVFIGPGNHDAARTALPQPGFYEEYAYPLLKLKNTTIISNPAVLDLHGRKFLIAHGRGIEDIVGNVPNTSHHEPARPMVELLKLRHLAPTFGGKVPVAPDPEDLLVIEEVPDFVQMGHVHVLDYQVYRGVFLLNSATWQAQTEFQKMVNIVPTPAKVPIVDVGTARLKTVVDFSKWCEGM

SEQ ID NO: 6 - protein, 677 aa, Thermococcus barophilus
MNELVKDLLSNRYLITPPAYFILSEYYKKSFTLAELIKFARTKNTFVIDERLAKEFLESKGLLSPLESFTKYTQKVPVETKAETPPSIAEVPMEISISGGSKEISIQSAVSGEKTAKLGKMVEIPPQASDKTEIAHDIQELPSEKGTSISTGEPMENMVSETISTEIVEEEPTIEGGESFISTGAPSPEEYTGAGDEENGVKMKAVYGDYGVLVPLEEEEIPAEERRAYSIYEDFKVELNESFEFVAKRIKVEYEIKFDVRNVKLKPPKAKNANGKEGEIVVKVYENYFKSRLKKLRRILRENPEVSGVIDIGKLSYINPDGEVTIIGLVNSKRETKKGFIFEVEDMTGIVKVFLGRDNEDYKKAFEVLPDSVVAFRGYYSKRGILFASKLYLPDVPIYKREKPPLEEKVYAVLISDIHVGSTKFCEKAFMKFLEWLNGNVESREEEEIVSRIKYMIIAGDVVDGIGIYPGQYNELAIPDIFDQYEALANLLANVPDHITMFIGPGNHDAARTALPQPEFYREYAKPLYELKNAVIISNPAVINLHGRDFLIVHGRGIEDVVSYIPGKSHLHPVEPMIELLKLRHVAPTFGGKVPIAPDPEDLLVIEEVPDLVQMGHVHVLDYQIYRGVFLINSATWQAQTEFQKMVNIVPTPARVPIIDIETARLKTVVDFSRWCE

SEQ ID NO: 7 - protein, 613 aa, Pyrococcus furiosus
MDEFVKSLLKANYLITPSAYYLLREYYEKGEFSIVELVKFARSRESYIITDALATEFLKVKGLEPILPVETKGGFVSTGESQKEQSYEESFGTKEEISQEIKEGESFISTGSEPLEEELNSIGIEEIGANEELVSNGNDNGGEAIVFDKYGYPMVYAPEEIEVEEKEYSKYEDLTIPMNPDFNYVEIKEDYDVVFDVRNVKLKPPKVKNGNGKEGEIIVEAYASLFRSRLKKLRKILRENPELDNVVDIGKLKYVKEDETVTIIGLVNSKREVNKGLIFEIEDLTGKVKVFLPKDSEDYREAFKVLPDAVVAFKGVYSKRGILYANKFYLPDVPLYRRQKPPLEEKVYAILISDIHVGSKEFCENAFIKFLEWLNGNVETKEEEEIVSRVKYLIIAGDVVDGVGVYPGQYADLTIPDIFDQYEALANLLSHVPKHITMFIAPGNHDAARQAIPQPEFYKEYAKPIYKLKNAVIISNPAVIRLHGRDFLIAHGRGIEDVVGSVPGLTHHKPGLPMVELLKMRHVAPMFGGKVPIAPDPEDLLVIEEVPDVVHMGHVHVYDAVVYRGVQLVNSATWQAQTEFQKMVNIVPTPAKVPVVDIDTAKVVKVLDFSGWC

SEQ ID NO: 8 - protein, 1291 aa, Thermococcus nautili
MGEELYSPEMKAYFESLQREIDRAYEIARKARSQGKDPSLDVEVPQATDMAGRVESLVGPPGVAERIRELVKEYGKELAALKVVDEIIEGKFGDLGSKEKYAEQAVRTALAILTEGIVSAPLEGIADVKIKRNTWADNSEYLALYYAGPIRSSGGTAQALSVLVGDYVRKKLGLDRFKPSEEHIERMVEEIDLYHRAVTRLQYHPEADEVRLAMRNIPIEITGEETDKVEVSHRNVPGVETNHLRGGAILVLAEGVLQKAKKLVKYIDKMGVEGWDWIKEFVEAKEKGKSSEEPKVDESKAEDSGAREEVAEKVEKGFYYELYERFRANIAPNKKYTKEIIGGRPLFAEPSENGGFRLRYGRSRVSGFATWSVNPATMLILDEFIAIGTQMKTERPGKGCIVTPATTVEGPIVKLKDGSVVRVDDYETALRVKNEIEEILYVGDALVNFGDFVENNQTLLPANYVEEWWVQELVKAIEELYEVELKPFSDNPREAVEEAAEYLEVDPDFLESLLRDPLRVKPSVELAIHLSKVLDIPFHPYYTLYWNTLKPEEVEELQKALLNAQIEWDEFRKLKFAKRVILENDPQIKRYLELLGLPHRLERTEDRRKVIVIDYPWSSALLTPLGDLEWEFKAKPFFTVIDIINESNPIKLRDRGISWIGARMGRPEKAKERKMKPPVQVLFPIGLAGGQSRDIKKAAEEGKTAKVEIAFFKCPKCGHTGPEHLCPVCGTRKELLWHCPKCNVDYPESEAKEFGFRCPRCDVELKPYAEREIKPSELLRRAMDNVKVYGIDRLKGVKGMTSGYKMAEPLEKGLLRVKNDVYVFKDGTIRFDATDAPITHFKPKEIGVSVEKLRELGYTHDFEGKPLERDDQIVELKVQDIILSYEAGKYLLKVARFVDDLLEKFYGLPRFYNAEKMEDLIGHLVIGLAPHTSAGIIGRIIGFSDVLVGYAHPYYHAAKRRNCDGDEDAVMLLLDALLNFSKYYLPEKRGGKMDAPLVVTTRLDPREVDSEVHNMDVVRYYPLEFYEVTYDMKSPKEIKFIERVEDRLGKPEMYEGIKFTHDTDDIGLGPKMSLYKQLGDMVEKVERQLALAERIRAVDEHHVAETILNSHLIPDLRGNLRSFTRQEFRCVKCNTKYRRPPLIGRCPKCGGKIVLTVSKGAVEKYLPTAKMLVTRYKVKDYTRQRICLTEKDIKTLFETVLPEKQRTLLGFSADVCEKMIKERTGSSNGKNGYLDGFNGKNGKAKKAKVGSEAKKEKKPKTEKSKDLKKEVKKEKAKARRKRISLDEFFGG

SEQ ID NO: 9 - protein, 1324 aa, Thermococcus kodakarensis
MSEEIYSPEMKAYFESLQREIDRAYAIARKARAQGKDPSFDVEVPQATDMAGRVESLVGPPGVAERIRELVKEYGKEIAALKVVDEIIEGKFGDLGSKEKYAEQAVRTALAILTEGIVSAPLEGIADVKIKRNEWADGSEYLALYYAGPIRSSGGTAQALSVLVGDYVRRKLGLDRFKPSDEHIERMVEEVDLYHRAVTRLQYHPEADEVRLAMRNIPIEITGEETDKVEVSHRNVPGVETNHLRGGAILVLAEGVLQKAKKLVKYIDKMGIEGWDWIKEFVEAKEKGKSSEENKDESKAEDTGTESVAEKKENVEKGFYYELYEKFRANIAPNKKYTKEIIGGRPLFAEPSTNGGFRLRYGRSRVSGFATWSVNPATMLILDEFIAIGTQMKTERPGKGCIVTPATTVEGPIVRLKNGSVVRVDDYETALKVRNEVDEILYVGDALVNFGDFVENNQTLLPANYVEEWWVQELVQAIKDLYEVELQPFAENDREAVEEAAEYLEVDPDFLWNLLKDPLRVKPDVETAIHLSTVLDIPFHPYYTLYWNTLQPEEVEELQKALLGAQIEWAEFRKNRFAKKVVLENDKNIKRYLELLGLPHRLERVEKKRKVIVVEYPWSAALLTPLGNLEWEFKAKPFYTVIDIINENNRIKLRDRGISWIGARMGRPEKAKERKMKPPVQVLFPIGLAGGQSRDIKKAAEEGKTARVEIAFFKCPKCGHVGPEHLCPVCGTRKELLWHCPKCGADYPESDAKDFNYRCPKCDVELKPYAEREIKPADLLRQAMDNVKVYGIDRLKGVKGMTSGYKMAEPLEKGLLRVKNDVYVFKDGTIRFDATDAPITHFKPKEIGTSVEKLRELGYTHDFEGKPLERDDQILELKVQDVILPYEAGRYLLKVARFIDDLLEKFYGLPRFYNAEKMEDLVGHLVIGLAPHTSAGIIGRIIGFSDVLVGYAHPYYHAAKRRNCDGDEDAVMLLLDALLNFSKYYLPEKRGGKMDAPLVVTTRLDPREVDSEVHNMDVVRYYPLEFYKATYELKSPKEVKVIERVEDRLGKPEMYEGIKFTHDTDDIGLGPKMSLYKQLGDMEEKVARQLALAERIRAVDEHHVAETIINSHLVPDLRGNLRSFTRQEFRCVKCNTKYRRPPLTGKCPKCGGKIVLTVSKGAIEKYLPTAKMLVTKYRVKDYTRQRICITEKDIKTLFENVFPEKQRTLMGFSADICEKMVKERTGHSNGKNGYLDEFNGKNGKASKKSGSLASKLSGKGKEPSKKKESAKPKRSEKVKNLTSFEAAAKNEQARGTAGNAKKAESEKPKRKKRKGISLDEFFGS

SEQ ID NO: 10 - protein, 1262 aa, Palaeococcus_ferrophilus
MTELYSDEMKAYFESLQREIDRAYEIAEKARAQGKDPVPEIEVPQATDMAGRVESLVGPKGVAERIRALVKEHGKELAALKIVDEIIEGRFGELGDEETLAEQAVRTSLAVLTEGIVSAPLEGIAFVKVRQNFDGSRYLALYYAGPIRSSGGTAQALSVLVGDYVRKKLGLDRYKPTPEEIERYVEEVDLYHRAVSRLQYHPSADEVRLAIKSIPVEITGEATDQVEVSGYRNLPRVETNQLRGGAILVLAEGVLQKAKKLLKYIDKLEVEGWEWLREFVESKESGKGKEKQEVVEESAEEETTAEVEMGFYYRLYEEFRKKIAPNKKYTKEIIGGRPLFAEPSTNGGFRLRYGRSRTSGFATWSVHPATMLLLDEFIAVGTQMKTERPGKGCITTPATTIEGPIVKLRDGSVVRVEDYETALRVRDSIEEILYVGDALVNFGDFVENNQTLLPANYAEEWWIQEFVKAVKDVYEVELKPFEENDGEDVEEAAEYLDVEPDFLGRLLYDPLRVRPRVEEAIHFSRVLRIPLHPYYTLYWNTLGVEELLALQEGLVDASIEWDSFRGVKFAKRLTTRNFSIKRSLELLGLPHRVENGEIVVEYPWSAALLTPLLNLERRLEPKPFHTPIDIINAVSDVKLRDRGMSWIGARMGRPEKAKERKMKPPVHVLFPIGLAGGSSRDIYKAFQEGKSTTVEIARFKCPSCGHVDIFPRCPKCGSRTEHLYTCPSCGHTSTEERVCPKCKVEMKPYAKTTFKIGDYLRPAMDNVKVYTLDRLKGVLGMSSAFKLAEPLEKGLLRAKNDVYVFKDGTIRFDATDAPVTHFRPREIGTSVEKLRELGYTHDFEGKPLESEDQILELKVQDVILPKTAGEYLLKVANFIDDLLERFYGLPRFYNAEKMEDLVGHLVIGLAPHTSAGIMGRIIGFVDALVGYAHPYYHAAKRRNCDGDEDAVMLLLDALLNFSKYYLPEKRGGKMDAPLVVTTRLDPREVDSEVHNMDVVRYYPLEFYRATYELKSPKEVKVIERVEDRLGKPEMYEGIKFTHDSDDIALGPKMSLYKQLGDMEDKVRRQLDLAKRIRAVDEHHVAETIINSHLIPDLRGNLRSFTRQEFRCVKCNTKYRRPPMSGKCPKCGGKIVLTVSKGAIEKYLSTAKMLVTEFDVLDYTRQRICITEKDIDTLFTNVFPRVQTTLMVATPKDVCSTMIARRRGTLKEKPKKSNGKSWDPSKDFKKPNGEAPKNGESKPAKAPKKKKVIGLDEFFGKKS

SEQ ID NO: 11 - protein, 1281 aa, Thermococcus barophilus
MSELYSEEMKAYFEMLQKEIDKAYEIAKKARAQGKDPVRDVEIPQATDMAGRVESLVGPKGVAERIRELAREYGKELAALKIVDEIIEGKFGKFDSKEKLAEQAVRTALAILTEGIVSAPIEGIAQVKIKKNPDGTEYLALYYAGPIRSSGGTAQALSVLVGDYVRKKLGLDKFKPTEKHIERMVEEVDLYHRAVTRLQYHPSADEVRLAMQNIPVEITGEATDDVEVSHRDVPGVETNQLRGGAILVLAEGVLQKAKKLVKYIDKMGIEGWGWLKEFVEAKEKGKTEEGTEEKAEKSKGEEAEIQTSVKVKKGFYYELYEKFRANIAPNNKYTKEIIGGRPLFAEPSENGGFRLRYGRSRVSGFATWSINPAVMILVDEFLAVGTQMKTERPGKGCIVTPATTAEGPIVKLKDGSVIRVDDYYLALKIRDQVEEILYLGDAIIAFGDFVENNQTLLPANYVEEWWIQEFVKAVEDIYEVPLKPFAENDEETVEEAADYLDLKPGFLAELLKDPLKIRPKVEEAIHLSKVLDIPLHPYYTLYWNTLKPEEVAELQSLLVNAEIEWDKYLGTKYAKKIVINLEVLGREGKRYLELLGLPHRVEDKSVIIEYPWSAALLTPLANLEKKFEPKEFHTTIDIVNEVSEVKLRDRGISWIGARMGRPEKAKERKMKPPVHVLFPIGLAGGSSRDIYKAAQEGKLAEVEIAQFKCTNCGHIDIFPTCPVCGAEAKLLYRCPKCNYSSTEETICPKCGIEMRTYVKKTINPSHLVKQAMQNVRVNTLDKLKGVMGMTSAHKVPEPLEKGILRAKNDVYVFKDGTIRFDATDAPITHFKPREIGVSVEKLRELGYTHDFEGKPLVSEDQIVELKVQDVILSKEAGKYLVRVAKFIDDLLERFYGLPRFYNVEKMEDLIGHLVIGLAPHTSAGIIGRIIGFVDALVGYAHPYYHAAKRRNCDGDEDSVMLLMDALLNFSRYYLPEKRGGKMDAPLVVTTRLDPREVDSEVHNMDIARYYPLEFYEATYELKSPKELVGVVERVEDRLGKPEMYEGLKFTHDTDDIALGPKLSKYKQLGDMEEKVRQQLALAEKIRAVNEHHVAETIINSHLIPDLRGNLRSFTRQEFRCVKCNTKYRRPPLSGKCPKCGGKIVLTVSKGAIEKYLPTAKLLVTNYDVLTYTRQRICLTEKDIKSLFANVFPETQRTLLALNTNDICDRMIAERTGKIVTKNGYLDEFHGNGRKVQKKVEAPKKVEKRELTKMQAHKSEKPGKHKPKKKKKVVSLDEFFGA

SEQ ID NO: 12 - protein, 1263 aa, Pyrococcus furiosus
MELPKEIEEYFEMLQREIDKAYEIAKKARSQGKDPSTDVEIPQATDMAGRVESLVGPPGVAQRIRELLKEYDKEIVALKIVDEIIEGKFGDFGSKEKYAEQAVRTALAILTEGIVSAPLEGIADVKIKRNTWADNSEYLALYYAGPIRSSGGTAQALSVLVGDYVRRKLGLDRFKPSGKHIERMVEEVDLYHRAVSRLQYHPSPDEVRLAMRNIPIEITGEATDDVEVSHRDVEGVETNQLRGGAILVLAEGVLQKAKKLVKYIDKMGIDGWEWLKEFVEAKEKGEEIEESESKAEESKVETRVEVEKGFYYKLYEKFRAEIAPSEKYAKEIIGGRPLFAGPSENGGFRLRYGRSRVSGFATWSINPATMVLVDEFLAIGTQMKTERPGKGAVVTPATTAEGPIVKLKDGSVVRVDDYNLALKIRDEVEEILYLGDAIIAFGDFVENNQTLLPANYVEEWWIQEFVKAVNEAYEVELRPFEENPRESVEEAAEYLEVDPEFLAKMLYDPLRVKPPVELAIHFSEILEIPLHPYYTLYWNTVNPKDVERLWGVLKDKATIEWGTFRGIKFAKKIEISLDDLGSLKRTLELLGLPHTVREGIVVVDYPWSAALLTPLGNLEWEFKAKPFYTVIDIINENNQIKLRDRGISWIGARMGRPEKAKERKMKPPVQVLFPIGLAGGSSRDIKKAAEEGKIAEVEIAFFKCPKCGHVGPETLCPECGIRKELIWTCPKCGAEYTNSQAEGYSYSCPKCNVKLKPFTKRKIKPSELLNRAMENVKVYGVDKLKGVMGMTSGWKIAEPLEKGLLRAKNEVYVFKDGTIRFDATDAPITHFRPREIGVSVEKLRELGYTHDFEGKPLVSEDQIVELKPQDVILSKEAGKYLLRVARFVDDLLEKFYGLPRFYNAEKMEDLIGHLVIGLAPHTSAGIVGRIIGFVDALVGYAHPYFHAAKRRNCDGDEDSVMLLLDALLNFSRYYLPEKRGGKMDAPLVITTRLDPREVDSEVHNMDVVRYYPLEFYEATYELKSPKELVGVIERVEDRLGKPEMYYGIKFTHDTDDIALGPKMSLYKQLGDMEEKVKRQLTLAERIRAVDQHYVAETILNSHLIPDLRGNLRSFTRQEFRCVKCNTKYRRPPLDGKCPVCGGKIVLTVSKGAIEKYLGTAKMLVANYNVKPYTRQRICLTEKDIDSLFEYLFPEAQLTLIVDPNDICMKMIKERTGETVQGGLLENFNSSGNNGKKIEKKEKKAKEKPKKKKVISLDDFFSKR

SEQ ID NO: 13 - protein, 476 aa, synthetic construct
  REGION 1..476: note = Synthetic polypeptide (DP1- deltaN(1-144))
MGENGESVVVLDKYGYPILYAPEEIGEEKEYSKYEDVVIEWNPSVTPVQIEKNYEVKFDVRQVKLRPPKVKNGSGKEGEIIVEAYASLFKSRLSKLKRILRENPEISNVVDIGKLNYVSGDEEVTIIGLVNSKRETNRGLIFEVEDKTGIVKVFLPKDSEDYREAFKVLPDAVVAFKGFYSKKGIFFANKFYLPDVPLYRKQKPPLEEKVYAILISDIHVGSREFCEKAFLKFLEWLNGHVESKEEEEIVSRVKYLIIAGDVVDGIGIYPGQYSDLVIPDIFDQYEALANLLANVPEHITMFIGPGNHDAARPAIPQPEFYKEYAKPIYKLKNAIIISNPAVIRLHGRDFLIAHGRGIEDVVSFVPGLTHHKPGLPMVELLKMRHLAPTFGGKVPIAPDPEDLLVIEEVPDLVQMGHVHVYDAVVYRGVQLVNSATWQAQTEFQKMVNIVPTPAKVPVVDVESARVVKVLDFSGWC

SEQ ID NO: 14 - protein, 476 aa, synthetic construct
  REGION 1..476: note = synthetic polypeptide (DP1- delta N(1-144)-H451A)
MGENGESVVVLDKYGYPILYAPEEIGEEKEYSKYEDVVIEWNPSVTPVQIEKNYEVKFDVRQVKLRPPKVKNGSGKEGEIIVEAYASLFKSRLSKLKRILRENPEISNVVDIGKLNYVSGDEEVTIIGLVNSKRETNRGLIFEVEDKTGIVKVFLPKDSEDYREAFKVLPDAVVAFKGFYSKKGIFFANKFYLPDVPLYRKQKPPLEEKVYAILISDIHVGSREFCEKAFLKFLEWLNGHVESKEEEEIVSRVKYLIIAGDVVDGIGIYPGQYSDLVIPDIFDQYEALANLLANVPEHITMFIGPGNADAARPAIPQPEFYKEYAKPIYKLKNAIIISNPAVIRLHGRDFLIAHGRGIEDVVSFVPGLTHHKPGLPMVELLKMRHLAPTFGGKVPIAPDPEDLLVIEEVPDLVQMGHVHVYDAVVYRGVQLVNSATWQAQTEFQKMVNIVPTPAKVPVVDVESARVVKVLDFSGWC

SEQ ID NO: 15 - protein, 1216 aa, synthetic construct
  REGION 1..1216: note = synthetic polypeptide (DP2-delta C(1217-1270) construct)
MELPKEMEEYFEMLQREIDKAYEIAKKARAQGKDPSLDVEIPQATDMAGRVESLVGPPGVAKRIRELVKEYGKEIAALKIVDEIIEGKFGDLGSREKYAEQAVRTALAILTEGIVSAPIEGIANVKIKRNTWADNSEYLALYYAGPIRSSGGTAQALSVLVGDYVRRKLGLDRFKPSEKHIERMVEEVDLYHRAVTRLQYHPSPEEVRLAMRNIPIEITGEATDDVEVSHRDVPGVETNQLRGGAILVLAEGVLQKAKKLVKYIDKMGIEGWEWLKEFVEAKEKGEPKEEGKEESLAESTLEETKVEVDMGFYYSLYQKFKEEIAPSDKYAKEVIGGRPLFSDPSKPGGFRLRYGRSRASGFATWGINPATMILVDEFLAIGTQLKTERPGKGAVVTPVTTIEGPIVKLKDGSVLRVDDYNLALKVREDVEEILYLGDAVIAFGDFVENNQTLLPANYCEEWWILEFVKALKEIYEVHLEPFTENEEESIEEASDYLEIDPEFLKEMLRDPLRVKPPVELAIHFSEVLGIPLHPYYTLYWNSVEPKDVEKLWRLLKNYAEIEWSNFRGIKFAKKIVISQEKLGDSKRTLELLGLPHTVRDGNVIVDYPWAAALLTPLGNLNWEFMAKPLYATIDIINENNEIKLRDRGISWIGARMGRPEKAKERKMKPPVQVLFPIGLAGGSSRDIKKAAEEGKVAEVEIAFFKCPKCGHVGPEHLCPNCGTRKELLWVCPRCNAEYPESQAEGYNYTCPKCNVKLRPYAKRKIRPSELLNRAMENVKVYGVDKLKGVMGMTSGWKMPEPLEKGLLRAKNDVYVFKDGTIRFDATDAPITHFRPREIGVSVEKLRELGYTHDFEGKPLVSEDQIVELKPQDIILSKEAGRYLLKVAKFVDDLLEKFYGLPRFYNAEKMEDLIGHLVIGLAPHTSAGIVGRIIGFVDALVGYAHPYFHAAKRRNCDGDEDAVMLLLDALLNFSRYYLPEKRGGKMDAPLVITTRLDPREVDSEVHNMDIVRYYPLEFYEATYELKSPKELVGVIERVEDRLGKPEMYYGLKFTHDTDDIALGPKMSLYKQLGDMEEKVRRQLEVAKRIRAVDEHGVAEKILNSHLIPDLRGNLRSFTRQEFRCVKCNTKFRRPPLNGKCPVCGGKIVLTVSKGAIEKYLGTAKMLVTEYNVKNYTRQRICLTERDIDSLFENVFPETQLTLIVNPNDICQRLVMAR

SEQ ID NO: 16 - protein, 662 aa, synthetic construct
  REGION 1..662: note = synthetic polypeptide (DP1 wt construct)
MGKHHHHSGHHHTGHHHHSGSHHHTSSSASTGENLYFQGTGDGSDELVKALERAGYLLTPSAYYLLVDHFKEGKFSLVELVKFAKSKGVFIIDGDLAYEFLQFLGLGVPQEIKESYISTGEEAEKTVESQETRASELEEGGVSQVSSGELQELKEESPEISTTEEEIGGLELVQSSISTGSEVEYNNGENGESVVVLDKYGYPILYAPEEIGEEKEYSKYEDVVIEWNPSVTPVQIEKNYEVKFDVRQVKLRPPKVKNGSGKEGEIIVEAYASLFKSRLSKLKRILRENPEISNVVDIGKLNYVSGDEEVTIIGLVNSKRETNRGLIFEVEDKTGIVKVFLPKDSEDYREAFKVLPDAVVAFKGFYSKKGIFFANKFYLPDVPLYRKQKPPLEEKVYAILISDIHVGSREFCEKAFLKFLEWLNGHVESKEEEEIVSRVKYLIIAGDVVDGIGIYPGQYSDLVIPDIFDQYEALANLLANVPEHITMFIGPGNHDAARPAIPQPEFYKEYAKPIYKLKNAIIISNPAVIRLHGRDFLIAHGRGIEDVVSFVPGLTHHKPGLPMVELLKMRHLAPTFGGKVPIAPDPEDLLVIEEVPDLVQMGHVHVYDAVVYRGVQLVNSATWQAQTEFQKMVNIVPTPAKVPVVDVESARVVKVLDFSGWC

SEQ ID NO: 17 - protein, 662 aa, synthetic construct
  REGION 1..662: note = synthetic polypeptide (DP1-H451A construct)
MGKHHHHSGHHHTGHHHHSGSHHHTSSSASTGENLYFQGTGDGSDELVKALERAGYLLTPSAYYLLVDHFKEGKFSLVELVKFAKSKGVFIIDGDLAYEFLQFLGLGVPQEIKESYISTGEEAEKTVESQETRASELEEGGVSQVSSGELQELKEESPEISTTEEEIGGLELVQSSISTGSEVEYNNGENGESVVVLDKYGYPILYAPEEIGEEKEYSKYEDVVIEWNPSVTPVQIEKNYEVKFDVRQVKLRPPKVKNGSGKEGEIIVEAYASLFKSRLSKLKRILRENPEISNVVDIGKLNYVSGDEEVTIIGLVNSKRETNRGLIFEVEDKTGIVKVFLPKDSEDYREAFKVLPDAVVAFKGFYSKKGIFFANKFYLPDVPLYRKQKPPLEEKVYAILISDIHVGSREFCEKAFLKFLEWLNGHVESKEEEEIVSRVKYLIIAGDVVDGIGIYPGQYSDLVIPDIFDQYEALANLLANVPEHITMFIGPGNADAARPAIPQPEFYKEYAKPIYKLKNAIIISNPAVIRLHGRDFLIAHGRGIEDVVSFVPGLTHHKPGLPMVELLKMRHLAPTFGGKVPIAPDPEDLLVIEEVPDLVQMGHVHVYDAVVYRGVQLVNSATWQAQTEFQKMVNIVPTPAKVPVVDVESARVVKVLDFSGWC

SEQ ID NO: 18 - protein, 519 aa, synthetic construct
  REGION 1..519: note = synthetic polypeptide (DP1- deltaN(1-144) construct)
MGKHHHHSGHHHTGHHHHSGSHHHTSSSASTGENLYFQGTGDGSGENGESVVVLDKYGYPILYAPEEIGEEKEYSKYEDVVIEWNPSVTPVQIEKNYEVKFDVRQVKLRPPKVKNGSGKEGEIIVEAYASLFKSRLSKLKRILRENPEISNVVDIGKLNYVSGDEEVTIIGLVNSKRETNRGLIFEVEDKTGIVKVFLPKDSEDYREAFKVLPDAVVAFKGFYSKKGIFFANKFYLPDVPLYRKQKPPLEEKVYAILISDIHVGSREFCEKAFLKFLEWLNGHVESKEEEEIVSRVKYLIIAGDVVDGIGIYPGQYSDLVIPDIFDQYEALANLLANVPEHITMFIGPGNHDAARPAIPQPEFYKEYAKPIYKLKNAIIISNPAVIRLHGRDFLIAHGRGIEDVVSFVPGLTHHKPGLPMVELLKMRHLAPTFGGKVPIAPDPEDLLVIEEVPDLVQMGHVHVYDAVVYRGVQLVNSATWQAQTEFQKMVNIVPTPAKVPVVDVESARVVKVLDFSGWC

SEQ ID NO: 19 - protein, 519 aa, synthetic construct
  REGION 1..519: note = synthetic polypeptide (DP1- deltaN(1-144)-H451A construct)
MGKHHHHSGHHHTGHHHHSGSHHHTSSSASTGENLYFQGTGDGSGENGESVVVLDKYGYPILYAPEEIGEEKEYSKYEDVVIEWNPSVTPVQIEKNYEVKFDVRQVKLRPPKVKNGSGKEGEIIVEAYASLFKSRLSKLKRILRENPEISNVVDIGKLNYVSGDEEVTIIGLVNSKRETNRGLIFEVEDKTGIVKVFLPKDSEDYREAFKVLPDAVVAFKGFYSKKGIFFANKFYLPDVPLYRKQKPPLEEKVYAILISDIHVGSREFCEKAFLKFLEWLNGHVESKEEEEIVSRVKYLIIAGDVVDGIGIYPGQYSDLVIPDIFDQYEALANLLANVPEHITMFIGPGNADAARPAIPQPEFYKEYAKPIYKLKNAIIISNPAVIRLHGRDFLIAHGRGIEDVVSFVPGLTHHKPGLPMVELLKMRHLAPTFGGKVPIAPDPEDLLVIEEVPDLVQMGHVHVYDAVVYRGVQLVNSATWQAQTEFQKMVNIVPTPAKVPVVDVESARVVKVLDFSGWC

SEQ ID NO: 20 - DNA, 1999 bp, synthetic construct
  misc_feature 1..1999: note = synthetic polynucleotide (DP1 wt construct)
  source 1..1999: mol_type = other DNA
ccatgggcaagcatcaccatcattcaggccatcaccataccggacaccaccatcattcaggcagtcatcaccatactagtagcagcgctagcaccggcgagaacctgtattttcaagggacgggggatggatccgatgaactggttaaagcactggaacgtgcaggttatctgctgaccccgagcgcatattacctgctggttgatcattttaaagagggcaaattttcactggtggaactggtgaaatttgccaaaagcaaaggcgtgtttattatcgatggtgatctggcctatgaattcctgcagtttttaggtctgggtgttccgcaagaaatcaaagaaagctatattagcaccggtgaagaagcagagaaaaccgttgaaagccaagaaacccgtgcaagcgaactggaagaaggtggcgttagccaggtgagcagcggtgaactgcaagaactgaaagaagaaagtccggaaattagcaccaccgaagaagaaattggcggtctggaattagttcagagcagcattagtaccggtagcgaagttgaatacaataatggtgaaaatggcgaaagcgttgtggtgctggataaatatggttatccgattctgtatgcaccggaagaaatcggcgaagaaaaagagtatagcaaatacgaagatgtggtgattgaatggaatccgagcgttacaccggttcagattgaaaagaattacgaggtgaaattcgatgtgcgtcaggttaaactgcgtccgcctaaagttaaaaatggtagcggtaaagaaggcgagattatcgttgaagcctatgcaagcctgtttaaaagccgtctgagcaaactgaaacgtattctgcgtgaaaacccggaaatttcaaatgtggtggatattggcaagctgaattatgttagcggtgatgaagaagtgacgattattggtctggttaatagcaaacgcgaaaccaatcgtggtctgatttttgaggttgaagataaaaccggcatcgtgaaagtttttctgccgaaagatagcgaagattatcgcgaagcatttaaagttctgccggatgcagttgttgcgtttaaaggtttttatagcaagaaaggcatcttcttcgccaacaaattttacctgcctgatgttccgctgtatcgtaaacagaaaccgcctctggaagagaaagtttatgcaattctgatcagcgatattcatgttggcagccgtgaattttgtgaaaaagcctttctgaaattcctggaatggctgaatggtcatgtggaatctaaagaagaagaggaaattgtcagtcgcgtcaagtatctgattattgccggtgatgttgttgatggcattggtatttatccgggtcagtatagcgatctggttatcccggatatttttgatcagtatgaagcactggcaaatctgctggcaaatgtgccggaacatatcaccatgtttattggtccgggtaatcatgatgcagcacgtccggcaattccgcagccggaattctataaagaatatgccaaaccgatctataaactgaaaaacgccatcatcattagcaatccggcagttattcgtctgcatggtcgtgattttctgattgcacatggtcgcggtattgaagatgttgttagctttgttccgggtctgacccatcataaaccgggtctgccgatggttgaactgctgaaaatgcgtcatctggcaccgacctttggtggtaaagttccgattgcacctgatccggaagatctgctggttattgaagaagttccggatctggttcagatgggtcatgttcatgtgtatgatgccgttgtttatcgtggtgttcagctggtgaatagcgcaacctggcaggcacagaccgaattccagaaaatggttaatattgttccgacaccggcaaaagttccggttgtggatgttgaaagcgcacgtgttgttaaagtgctggattttagcggttggtgctaagcggccgc

SEQ ID NO: 21 - DNA, 3822 bp, synthetic construct
  misc_feature 1..3822: note = synthetic polynucleotide (DP2 wt construct)
  source 1..3822: mol_type = other DNA
catatggagcttccaaaggaaatggaagagtatttcgaaatgctgcagcgtgagattgataaggcctatgagattgctaaaaaggcgcgtgctcagggtaaagatccgagcttggatgttgagattcctcaagctaccgatatggccggacgtgttgaaagcttagttggcccgccaggagttgctaaacgtattcgtgagttagttaaagagtatggaaaggagattgccgccctcaaaattgttgacgagattattgaaggtaaattcggcgaccttggaagtcgtgagaagtacgctgagcaggccgtgcgtactgcattggcaattctgaccgagggaattgtttccgctccgattgagggtattgctaacgtcaagattaagcgtaacacttgggctgacaactccgagtatctggccctctactatgcgggcccaattcgtagctcaggtggaaccgctcaggcccttagcgttttggttggtgattacgttcgtcgtaagctcggcctggaccgttttaagccaagcgagaagcacattgagcgtatggttgaagaggtggatctttatcatcgtgccgtcactcgtttgcagtatcatccgtctccagaggaagttcgtcttgctatgcgtaacattccaatcgagattacaggtgaggcaacggatgatgttgaagtctcccatcgtgacgttcctggggtcgaaactaatcagcttcgtggcggtgcaattctggttctggctgaaggtgtcctccaaaaggctaagaagcttgttaaatatattgacaagatggggattgagggttgggaatggctcaaggaattcgttgaggctaaggagaagggagaacctaaggaggaaggtaaagaggaaagcctggctgaatcaacccttgaggaaacgaaggtcgaagttgatatgggcttctactactctctttatcagaagttcaaggaggatatcgctccaagcgacaagtacgctaaggaagttattgggggccgtcctttattctctgatccatctaaaccaggtggatttcgtttgcgttacgggcgtagtcgtgcgagcgggttcgcaacttggggaattaaccctgcaaccatgattttagttgacgagttcttggcaattggaactcaactgaaaaccgaacgtccagggaagggtgcggtggttactccagtaacgacgattgagggcccaattgtcaagctcaaggatggtagcgttctgcgtgttgatgattataatttggccctcaaggttcgtgaagatgttgaggagattctttaccttggtgacgcggtgattgctttcggagatttcgttgagaacaaccaaaccctccttccagctaattattgcgaggagtggtggattcttgagttcgtcaaggccctcaaagagatttatgaggtccaccttgagccttttaccgaaaatgaggaagagagcattgaggaagcttctgattacctggagattgacccagaatttttaaaggaaatgctccgtgatccgttgcgtgtaaaacctccggttgagcttgccattcacttctccgaggttcttggaattccgttgcatccgtattacaccttgtactggaattccgtcgagccgaaagacgttgaaaagctgtggcgtttgctcaaaaattatgccgagattgaatggagcaactttcgtggaattaaatttgcgaagaaaattgtcatttcccaagaaaagcttggagattctaagcgtactttagagctcctgggattgcctcacaccgttcgtgatgggaacgttattgtggactatccgtgggctgccgccctcttaactccgctcggcaacttgaattgggagttcatggcaaagccgctttacgccaccattgatattatcaacgagaataacgagattaagcttcgtgatcgtggaattagctggattggcgcgcgtatgggtcgtccagagaaggcgaaagagcgtaagatgaagcctcctgttcaagttctgttcccaattggtctggccggcgggagcagtcgtgacattaagaaagccgctgaagagggtaaagtggccgaggttgagattgctttcttcaagtgcccgaagtgcggtcacgttggcccagagcatttatgtccaaactgtggaacgcgtaaagaacttctctgggtctgtccacgttgtaacgcggagtaccctgagagccaggccgagggctataattacacttgtccaaagtgcaacgtcaagctgcgtccgtacgccaagcgtaagattcgtccttccgaattgctgaatcgtgcgatggagaacgtcaaagtctacggagttgacaagttaaagggagtcatgggaatgacttctggctggaagatgccggagccacttgagaaaggtcttctgcgtgctaaaaacgatgtttacgtgttcaaggatggtaccattcgttttgacgcaacagatgccccaattacccacttccgtccgcgtgaaattggagtttccgttgagaagttacgtgaacttggttatactcatgacttcgagggtaaaccgctggttagcgaggatcaaattgttgagctcaagccgcaggatattatcctgtcaaaggaagccgggcgttatctcctgaaggtggccaagtttgttgatgacctccttgagaaattctacggcttgccgcgtttttacaacgccgaaaagatggaagatttaattggccatctcgtcattggcttggctccacacacctccgctggtattgtgggtcgtattattggctttgtcgatgccctggttggttacgctcacccgtacttccacgctgcgaagcgtcgtaactgtgatggagatgaagacgctgttatgctcctcctggatgccctcctgaacttctctcgctattaccttccggaaaagcgtggtggaaagatggacgctcctttggtaattacaacgcgtcttgatccgcgtgaagttgatagcgaagtccataacatggacattgtccgttattatccgctggagttctacgaggcaacttatgagcttaaatccccgaaggaactcgttggggtaattgaacgtgttgaggatcgcctgggaaagcctgagatgtactatggcctgaagttcacccacgatacggatgacattgctttgggtccaaagatgagtctgtacaagcagttaggggacatggaagagaaggtccgtcgtcagcttgaagttgctaagcgtattcgtgctgtggatgaacatggagttgccgagaagattttgaattcccacctgattcctgacttacgtggtaatctgcgtagctttacgcgtcaagagttccgttgcgtcaagtgcaacactaagttccgtcgtccgccactgaatggaaagtgcccggtttgcggaggtaaaattgtccttaccgtgagcaagggggcaatcgagaaatacttgggaactgccaagatgctcgttaccgaatacaacgtgaagaattacacgcgtcagcgtatttgcttgactgaacgtgacattgattcactgttcgaaaacgtgttcccagagacccagttaactttaatcgttaatccaaatgatatttgccagcgtctcgtgatggcgcgtactggagaagttaataagtcaggcctccttgaaaatctttctaacggttcgaagaagactgagaaagctgaaaaggccgagaagccgcgtaagaaatcagatgagaagccaaagaagaaacgtgttatcagcctcgaagagttcttttcacgtaaatcaaagtagctcgag

SEQ ID NO: 22 - DNA, 1999 bp, synthetic construct
  misc_feature 1..1999: note = synthetic polynucleotide (DP1-H451A construct)
  source 1..1999: mol_type = other DNA
ccatgggcaagcatcaccatcattcaggccatcaccataccggacaccaccatcattcaggcagtcatcaccatactagtagcagcgctagcaccggcgagaacctgtattttcaagggacgggggatggatccgatgaactggttaaagcactggaacgtgcaggttatctgctgaccccgagcgcatattacctgctggttgatcattttaaagagggcaaattttcactggtggaactggtgaaatttgccaaaagcaaaggcgtgtttattatcgatggtgatctggcctatgaattcctgcagtttttaggtctgggtgttccgcaagaaatcaaagaaagctatattagcaccggtgaagaagcagagaaaaccgttgaaagccaagaaacccgtgcaagcgaactggaagaaggtggcgttagccaggtgagcagcggtgaactgcaagaactgaaagaagaaagtccggaaattagcaccaccgaagaagaaattggcggtctggaattagttcagagcagcattagtaccggtagcgaagttgaatacaataatggtgaaaatggcgaaagcgttgtggtgctggataaatatggttatccgattctgtatgcaccggaagaaatcggcgaagaaaaagagtatagcaaatacgaagatgtggtgattgaatggaatccgagcgttacaccggttcagattgaaaagaattacgaggtgaaattcgatgtgcgtcaggttaaactgcgtccgcctaaagttaaaaatggtagcggtaaagaaggcgagattatcgttgaagcctatgcaagcctgtttaaaagccgtctgagcaaactgaaacgtattctgcgtgaaaacccggaaatttcaaatgtggtggatattggcaagctgaattatgttagcggtgatgaagaagtgacgattattggtctggttaatagcaaacgcgaaaccaatcgtggtctgatttttgaggttgaagataaaaccggcatcgtgaaagtttttctgccgaaagatagcgaagattatcgcgaagcatttaaagttctgccggatgcagttgttgcgtttaaaggtttttatagcaagaaaggcatcttcttcgccaacaaattttacctgcctgatgttccgctgtatcgtaaacagaaaccgcctctggaagagaaagtttatgcaattctgatcagcgatattcatgttggcagccgtgaattttgtgaaaaagcctttctgaaattcctggaatggctgaatggtcatgtggaatctaaagaagaagaggaaattgtcagtcgcgtcaagtatctgattattgccggtgatgttgttgatggcattggtatttatccgggtcagtatagcgatctggttatcccggatatttttgatcagtatgaagcactggcaaatctgctggcaaatgtgccggaacatatcaccatgtttattggtccgggtaatgcggatgcagcacgtccggcaattccgcagccggaattctataaagaatatgccaaaccgatctataaactgaaaaacgccatcatcattagcaatccggcagttattcgtctgcatggtcgtgattttctgattgcacatggtcgcggtattgaagatgttgttagctttgttccgggtctgacccatcataaaccgggtctgccgatggttgaactgctgaaaatgcgtcatctggcaccgacctttggtggtaaagttccgattgcacctgatccggaagatctgctggttattgaagaagttccggatctggttcagatgggtcatgttcatgtgtatgatgccgttgtttatcgtggtgttcagctggtgaatagcgcaacctggcaggcacagaccgaattccagaaaatggttaatattgttccgacaccggcaaaagttccggttgtggatgttgaaagcgcacgtgttgttaaagtgctggattttagcggttggtgctaagcggccgc

SEQ ID NO: 23 - DNA, 1570 bp, synthetic construct
  misc_feature 1..1570: note = synthetic polynucleotide (DP1- deltaN(1-144) construct)
  source 1..1570: mol_type = other DNA
ccatgggcaagcatcaccatcattcaggccatcaccataccggacaccaccatcattcaggcagtcatcaccatactagtagcagcgctagcaccggcgagaacctgtattttcaagggacgggggatggatccggtgaaaatggcgaaagcgttgtggtgctggataaatatggttatccgattctgtatgcaccggaagaaatcggcgaagaaaaagagtatagcaaatacgaagatgtggtgattgaatggaatccgagcgttacaccggttcagattgaaaagaattacgaggtgaaattcgatgtgcgtcaggttaaactgcgtccgcctaaagttaaaaatggtagcggtaaagaaggcgagattatcgttgaagcctatgcaagcctgtttaaaagccgtctgagcaaactgaaacgtattctgcgtgaaaacccggaaatttcaaatgtggtggatattggcaagctgaattatgttagcggtgatgaagaagtgacgattattggtctggttaatagcaaacgcgaaaccaatcgtggtctgatttttgaggttgaagataaaaccggcatcgtgaaagtttttctgccgaaagatagcgaagattatcgcgaagcatttaaagttctgccggatgcagttgttgcgtttaaaggtttttatagcaagaaaggcatcttcttcgccaacaaattttacctgcctgatgttccgctgtatcgtaaacagaaaccgcctctggaagagaaagtttatgcaattctgatcagcgatattcatgttggcagccgtgaattttgtgaaaaagcctttctgaaattcctggaatggctgaatggtcatgtggaatctaaagaagaagaggaaattgtcagtcgcgtcaagtatctgattattgccggtgatgttgttgatggcattggtatttatccgggtcagtatagcgatctggttatcccggatatttttgatcagtatgaagcactggcaaatctgctggcaaatgtgccggaacatatcaccatgtttattggtccgggtaatcatgatgcagcacgtccggcaattccgcagccggaattctataaagaatatgccaaaccgatctataaactgaaaaacgccatcatcattagcaatccggcagttattcgtctgcatggtcgtgattttctgattgcacatggtcgcggtattgaagatgttgttagctttgttccgggtctgacccatcataaaccgggtctgccgatggttgaactgctgaaaatgcgtcatctggcaccgacctttggtggtaaagttccgattgcacctgatccggaagatctgctggttattgaagaagttccggatctggttcagatgggtcatgttcatgtgtatgatgccgttgtttatcgtggtgttcagctggtgaatagcgcaacctggcaggcacagaccgaattccagaaaatggttaatattgttccgacaccggcaaaagttccggttgtggatgttgaaagcgcacgtgttgttaaagtgctggattttagcggttggtgctaagcggccgc

SEQ ID NO: 24 - DNA, 1570 bp, synthetic construct
  misc_feature 1..1570: note = synthetic polynucleotide (DP1- deltaN(1-144)-H451A construct)
  source 1..1570: mol_type = other DNA
ccatgggcaagcatcaccatcattcaggccatcaccataccggacaccaccatcattcaggcagtcatcaccatactagtagcagcgctagcaccggcgagaacctgtattttcaagggacgggggatggatccggtgaaaatggcgaaagcgttgtggtgctggataaatatggttatccgattctgtatgcaccggaagaaatcggcgaagaaaaagagtatagcaaatacgaagatgtggtgattgaatggaatccgagcgttacaccggttcagattgaaaagaattacgaggtgaaattcgatgtgcgtcaggttaaactgcgtccgcctaaagttaaaaatggtagcggtaaagaaggcgagattatcgttgaagcctatgcaagcctgtttaaaagccgtctgagcaaactgaaacgtattctgcgtgaaaacccggaaatttcaaatgtggtggatattggcaagctgaattatgttagcggtgatgaagaagtgacgattattggtctggttaatagcaaacgcgaaaccaatcgtggtctgatttttgaggttgaagataaaaccggcatcgtgaaagtttttctgccgaaagatagcgaagattatcgcgaagcatttaaagttctgccggatgcagttgttgcgtttaaaggtttttatagcaagaaaggcatcttcttcgccaacaaattttacctgcctgatgttccgctgtatcgtaaacagaaaccgcctctggaagagaaagtttatgcaattctgatcagcgatattcatgttggcagccgtgaattttgtgaaaaagcctttctgaaattcctggaatggctgaatggtcatgtggaatctaaagaagaagaggaaattgtcagtcgcgtcaagtatctgattattgccggtgatgttgttgatggcattggtatttatccgggtcagtatagcgatctggttatcccggatatttttgatcagtatgaagcactggcaaatctgctggcaaatgtgccggaacatatcaccatgtttattggtccgggtaatgcggatgcagcacgtccggcaattccgcagccggaattctataaagaatatgccaaaccgatctataaactgaaaaacgccatcatcattagcaatccggcagttattcgtctgcatggtcgtgattttctgattgcacatggtcgcggtattgaagatgttgttagctttgttccgggtctgacccatcataaaccgggtctgccgatggttgaactgctgaaaatgcgtcatctggcaccgacctttggtggtaaagttccgattgcacctgatccggaagatctgctggttattgaagaagttccggatctggttcagatgggtcatgttcatgtgtatgatgccgttgtttatcgtggtgttcagctggtgaatagcgcaacctggcaggcacagaccgaattccagaaaatggttaatattgttccgacaccggcaaaagttccggttgtggatgttgaaagcgcacgtgttgttaaagtgctggattttagcggttggtgctaagcggccgc

SEQ ID NO: 25 - DNA, 3660 bp, synthetic construct
  misc_feature 1..3660: note = synthetic polynucleotide (DP2- deltaC(1217-1270) construct)
  source 1..3660: mol_type = other DNA
catatggagcttccaaaggaaatggaagagtatttcgaaatgctgcagcgtgagattgataaggcctatgagattgctaaaaaggcgcgtgctcagggtaaagatccgagcttggatgttgagattcctcaagctaccgatatggccggacgtgttgaaagcttagttggcccgccaggagttgctaaacgtattcgtgagttagttaaagagtatggaaaggagattgccgccctcaaaattgttgacgagattattgaaggtaaattcggcgaccttggaagtcgtgagaagtacgctgagcaggccgtgcgtactgcattggcaattctgaccgagggaattgtttccgctccgattgagggtattgctaacgtcaagattaagcgtaacacttgggctgacaactccgagtatctggccctctactatgcgggcccaattcgtagctcaggtggaaccgctcaggcccttagcgttttggttggtgattacgttcgtcgtaagctcggcctggaccgttttaagccaagcgagaagcacattgagcgtatggttgaagaggtggatctttatcatcgtgccgtcactcgtttgcagtatcatccgtctccagaggaagttcgtcttgctatgcgtaacattccaatcgagattacaggtgaggcaacggatgatgttgaagtctcccatcgtgacgttcctggggtcgaaactaatcagcttcgtggcggtgcaattctggttctggctgaaggtgtcctccaaaaggctaagaagcttgttaaatatattgacaagatggggattgagggttgggaatggctcaaggaattcgttgaggctaaggagaagggagaacctaaggaggaaggtaaagaggaaagcctggctgaatcaacccttgaggaaacgaaggtcgaagttgatatgggcttctactactctctttatcagaagttcaaggaggatatcgctccaagcgacaagtacgctaaggaagttattgggggccgtcctttattctctgatccatctaaaccaggtggatttcgtttgcgttacgggcgtagtcgtgcgagcgggttcgcaacttggggaattaaccctgcaaccatgattttagttgacgagttcttggcaattggaactcaactgaaaaccgaacgtccagggaagggtgcggtggttactccagtaacgacgattgagggcccaattgtcaagctcaaggatggtagcgttctgcgtgttgatgattataatttggccctcaaggttcgtgaagatgttgaggagattctttaccttggtgacgcggtgattgctttcggagatttcgttgagaacaaccaaaccctccttccagctaattattgcgaggagtggtggattcttgagttcgtcaaggccctcaaagagatttatgaggtccaccttgagccttttaccgaaaatgaggaagagagcattgaggaagcttctgattacctggagattgacccagaatttttaaaggaaatgctccgtgatccgttgcgtgtaaaacctccggttgagcttgccattcacttctccgaggttcttggaattccgttgcatccgtattacaccttgtactggaattccgtcgagccgaaagacgttgaaaagctgtggcgtttgctcaaaaattatgccgagattgaatggagcaactttcgtggaattaaatttgcgaagaaaattgtcatttcccaagaaaagcttggagattctaagcgtactttagagctcctgggattgcctcacaccgttcgtgatgggaacgttattgtggactatccgtgggctgccgccctcttaactccgctcggcaacttgaattgggagttcatggcaaagccgctttacgccaccattgatattatcaacgagaataacgagattaagcttcgtgatcgtggaattagctggattggcgcgcgtatgggtcgtccagagaaggcgaaagagcgtaagatgaagcctcctgttcaagttctgttcccaattggtctggccggcgggagcagtcgtgacattaagaaagccgctgaagagggtaaagtggccgaggttgagattgctttcttcaagtgcccgaagtgcggtcacgttggcccagagcatttatgtccaaactgtggaacgcgtaaagaacttctctgggtctgtccacgttgtaacgcggagtaccctgagagccaggccgagggctataattacacttgtccaaagtgcaacgtcaagctgcgtccgtacgccaagcgtaagattcgtccttccgaattgctgaatcgtgcgatggagaacgtcaaagtctacggagttgacaagttaaagggagtcatgggaatgacttctggctggaagatgccggagccacttgagaaaggtcttctgcgtgctaaaaacgatgtttacgtgttcaaggatggtaccattcgttttgacgcaacagatgccccaattacccacttccgtccgcgtgaaattggagtttccgttgagaagttacgtgaacttggttatactcatgacttcgagggtaaaccgctggttagcgaggatcaaattgttgagctcaagccgcaggatattatcctgtcaaaggaagccgggcgttatctcctgaaggtggccaagtttgttgatgacctccttgagaaattctacggcttgccgcgtttttacaacgccgaaaagatggaagatttaattggccatctcgtcattggcttggctccacacacctccgctggtattgtgggtcgtattattggctttgtcgatgccctggttggttacgctcacccgtacttccacgctgcgaagcgtcgtaactgtgatggagatgaagacgctgttatgctcctcctggatgccctcctgaacttctctcgctattaccttccggaaaagcgtggtggaaagatggacgctcctttggtaattacaacgcgtcttgatccgcgtgaagttgatagcgaagtccataacatggacattgtccgttattatccgctggagttctacgaggcaacttatgagcttaaatccccgaaggaactcgttggggtaattgaacgtgttgaggatcgcctgggaaagcctgagatgtactatggcctgaagttcacccacgatacggatgacattgctttgggtccaaagatgagtctgtacaagcagttaggggacatggaagagaaggtccgtcgtcagcttgaagttgctaagcgtattcgtgctgtggatgaacatggagttgccgagaagattttgaattcccacctgattcctgacttacgtggtaatctgcgtagctttacgcgtcaagagttccgttgcgtcaagtgcaacactaagttccgtcgtccgccactgaatggaaagtgcccggtttgcggaggtaaaattgtccttaccgtgagcaagggggcaatcgagaaatacttgggaactgccaagatgctcgttaccgaatacaacgtgaagaattacacgcgtcagcgtatttgcttgactgaacgtgacattgattcactgttcgaaaacgtgttcccagagacccagttaactttaatcgttaatccaaatgatatttgccagcgtctcgtgatggcgcgttaactcgag

SEQ ID NO: 26 - protein, 44 aa, synthetic construct
  REGION 1..44: note = synthetic peptide (polyhistidine tag+TEV cleavage site)
MGKHHHHSGHHHTGHHHHSGSHHHTSSSASTGENLYFQGTGDGS

SEQ ID NO: 27 - protein, 7 aa, synthetic construct
  REGION 1..7: note = synthetic peptide (TEV cleavage site)
ENLYFQG

SEQ ID NO: 28 - DNA, 34 bp, synthetic construct
  misc_feature 1..34: note = synthetic oligonucleotide (primer P2.6kb_Fwd)
  source 1..34: mol_type = other DNA
ctactactctctttatcagaagttcaaggaggat

SEQ ID NO: 29 - DNA, 28 bp, synthetic construct
  misc_feature 1..28: note = synthetic oligonucleotide (Primer P2.6kb_rev)
  source 1..28: mol_type = other DNA
cgattaaagttaactgggtctctgggaa

SEQ ID NO: 30 - DNA, 24 bp, synthetic construct
  misc_feature 1..24: note = synthetic oligonucleotide (primer 5'-6FAM)
  source 1..24: mol_type = other DNA
gaggtctcgctccgaccgctcccg

SEQ ID NO: 31 - DNA, 38 bp, synthetic construct
  misc_feature 1..38: note = synthetic oligonucleotide (Template DNA12)
  source 1..38: mol_type = other DNA
agtgcctaacgatgcgggagcggtcggagcgagacctc

SEQ ID NO: 32 - DNA, 38 bp, synthetic construct; identical to SEQ ID NO: 31
  misc_feature 1..38: note = synthetic oligonucleotide (Template RNA12)
  source 1..38: mol_type = other DNA

SEQ ID NO: 33 - DNA, 38 bp, synthetic construct; identical to SEQ ID NO: 31
  misc_feature 1..38: note = synthetic oligonucleotide (Template 2'-O-MeRNA12)
  source 1..38: mol_type = other DNA

SEQ ID NO: 34 - DNA, 62 bp, synthetic construct
  misc_feature 1..62: note = synthetic oligonucleotide (Template DNA36)
  source 1..62: mol_type = other DNA
agtgcctaaccaagtgcctaaccaagtgcctaacgatgcgggagcggtcggagcgagacctc

SEQ ID NO: 35 - DNA, 62 bp, synthetic construct; identical to SEQ ID NO: 34
  misc_feature 1..62: note = synthetic oligonucleotide (Template RNA36)
  source 1..62: mol_type = other DNA

(19) United States
(12) Patent Application Publication
(10) Pub. No.: US 2025/0297236 A1
(43) Pub. Date: Sep. 25, 2025

(54) IDENTIFYING THE MINIMAL CATALYTIC CORE OF DNA POLYMERASE D AND APPLICATIONS THEREOF

(71) Applicants: INSTITUT PASTEUR, PARIS CEDEX 15 (FR); CENTRE NATIONAL DE LA RECHERCHE SCIENTIFIQUE, PARIS (FR)

(72) Inventors: Ludovic SAUGUET, KREMLIN BICETRE (FR); Marc DELARUE, VERSAILLES (FR); Remi SIESKIND, CACHAN (FR); Clement MADRU, PARIS (FR)

(21) Appl. No.: 18/861,827

(22) PCT Filed: May 19, 2023
(86) PCT No.: PCT/EP2023/063452

§ 371 Date: Oct. 30, 2024

(30) Foreign Application Priority Data
May 19, 2022 (EP) 22305743

(51) Int. Cl. C12N 9/12; C12P 19/34; C12Q 1/6844

(52) U.S. Cl. CPC C12N 9/1252; C12P 19/34; C12Q 1/6844; C12Y 207/07007

(57) ABSTRACT

The invention relates to an engineered DNA polymerase D (PolD) and its use for nucleic acid amplification including reverse transcription of RNA.

### FIELD OF THE INVENTION

The invention relates to an engineered DNA polymerase D (PolD) and its use for nucleic acid amplification including reverse transcription of RNA.

### BACKGROUND OF THE INVENTION

DNA polymerases (DNAPs) are molecular motors directing the synthesis of DNA from nucleotides and a DNA template. On the basis of their amino acid sequence and structural analysis, DNAPs have been classified into seven families, A, B, C, D, X, Y and reverse transcriptases (Raia et al., Biochem. Soc. Trans., 2019, 28, 239-49). In addition to their fundamental biological functions, DNAPs are versatile tools used in important molecular biology core technologies. The best known DNAP-based biotechnology application is the polymerization chain reaction (PCR). The PCR reaction consists of an exponential amplification of a DNA template through multiple cycles (generally 20-30) of denaturation, primer annealing, and elongation by a polymerase. Performing PCR requires highly thermostable polymerase that display a sufficiently high specificity, processivity, fidelity and resistance to contaminants, thereby strongly restricting the repertoire of polymerases that are capable of PCR activity. As nucleic acid analysis by PCR moves toward clinical diagnostics and forensics, there is a constant need for DNAPs capable of amplifying DNA from more difficult clinical samples such as tissue, blood, body fluids.

Thermostable DNAPs marketed for PCR invariably are either family-A DNAPs from thermophilic and hyperthermophilic Bacteria, family-B and family-Y DNAPs from the hyperthermophilic Archaea. Recently, a novel family (D-family) of archaeal thermostable DNAP, named PolD, was discovered and shown to have significant commercial value in PCR technology (Killelea et al., Front. Microbiol., 2014, 5, 195). In particular, PolD from Pyrococcus abyssi showed not only greater resistance to high denaturation temperatures than the popular Taq during cycling, but also superior tolerance to the presence of potential inhibitors (including ions and detergents) and is completely resistant to haemoglobin. In addition, PolD shows among the highest tolerance to calcium ions compared to other thermostable DNAPs.

PolD is a major replicative DNA polymerase and is found in most Archaea. It is composed of a large catalytic subunit (DP2) with 5′-3′ DNA polymerase activity and a smaller subunit (DP1) with 3′-5′ proofreading exonuclease activity. The crystal and cryo-EM structures of PolD have been determined (Sauguet et al., Nature communications, 2016, 7, 12227; Raia et al., PLOS Biology, 2019, 18, 17 (1) e3000122; Madru et al., Nature communications, 2020, 27, 11 (1), 1591). DP1 structure shows a large calcineurin-like phosphodiesterase (PDE) domain which forms the nuclease catalytic core and a N-terminal region that is not needed for exonuclease activity. The PDE domain includes the insertion of an oligonucleotide/oligosaccharide (OB) binding domain in the N-terminal part and contains five conserved phosphodiesterase motifs, which form the nuclease active site. The N-terminal region is a HSH (helix-strand-helix or helix-span-helix) domain that interacts, in the cell, with other partners of the DNA replication machinery, including the replicative helicase. This domain is connected to the phosphodiesterase domain of DP1 by a flexible linker-domain. DP2 comprises three domains which form the polymerase catalytic core (N-terminal domain, central domain, and catalytic domain) and a C-terminal domain which interacts with other replication factors, including the DNA primase and the Proliferating Cell Nuclear Antigen (PCNA). DP1 and DP2 subunits are conserved, in particular in hyperthermophilic Archaea of the order Thermococcales, which include Pyrococcus, Thermococcus, and Palaeococcus. It was found that PolD is an atypical DNA polymerase whose catalytic core is structurally distinct from the Klenow-like catalytic core, which is shared by all other thermostable DNAPs marketed for PCR. Unlike other DNAPs used in PCR, which are all monomeric, PolD is heterodimeric and thus substantially larger than other DNAPs marketed for PCR.

Reverse transcriptase are specialized DNA polymerases, which are able to incorporate dNTPs into a DNA polymer by using a RNA template molecule. During the long process of natural evolution, most DNA polymerases acquired a very high specificity regarding both the templates and the substrates. Most DNA polymerases specifically polymerases dNTPs and use DNA templates. Polymerases present nevertheless a variable tolerance to substrate and template changes. Previous studies reported the capacity of PolD to incorporate up to 4 NTPs in a DNA polymer using a DNA template (Zatopek et al., Nucleic acids Research, 2020, 48, 12204-12218) and to incorporate a dNTP when encountering a template that contains a single RNA base (Lemor et al., J. Mol. Biol., 2018, 430, 4908-4924).

There is a need for more robust DNA polymerases that can be used in wide ranges of PCR applications. In addition, RNA amplification by PCR requires two different enzymes, a reverse transcriptase (RT) and a DNA polymerase. Therefore, a DNA polymerase having reverse transcriptase activity would be most advantageous.

### SUMMARY OF THE INVENTION

The inventors have identified and deleted domains of PolD, which are non-essential for the catalytic activity, resulting in a shorter version of the PolD polymerase, named PolD-catalytic-core (FIGS. 1 and 2). They have shown that this construct is expressed readily in E. coli and is a fully active DNA polymerase compared to full-length PolD (FIG. 4). Furthermore, they have shown that at higher concentrations of polymerase, the engineered PolD remains active while the activity of full-length PolD is inhibited (FIG. 5). Therefore, the PolD-catalytic-core constructions remain active in a wider range of PCR conditions and can therefore be used for a wider range of PCR applications. Furthermore, the inventors have discovered that PolD is capable of reverse-transcriptase activity, meaning that it is capable of polymerizing DNA by using RNA as a template (FIG. 6). This finding was unexpected as PolD is a replicative DNA-dependent DNA polymerase. This novel activity is very important as PolD can be used to amplify a specific DNA sequence by starting from an RNA template, which has interesting applications, in particular for the detection of RNA viruses such as SARS-COV2 and others. Finally, they have found that PolD exonuclease-deficient variants show a more efficient reverse-transcriptase activity than the wild-type (FIG. 6). Due to the high degree of conservation of PolD (FIGS. 1 and 2), new PolD constructs with improved activities can be obtained from various Archaea, in particular thermostable PolD from hyperthermophilic Archaea of the order Thermococcales.

One aspect of the invention relates to an engineered DNA polymerase of the family D (PolD) comprising: (i) a truncated subunit DP1 comprising a N-terminal deletion of at least the helix-strand-helix (HSH) domain and (ii) a truncated subunit DP2 comprising a C-terminal deletion of at least 50 amino acids.

In some embodiments of the engineered PolD according to the invention, the N-terminal deletion of DP1 is at least from positions 1 to 67; preferably from position 1 to any one of positions 67 to 196; more preferably from positions 1 to 144 or 1 to 196, the indicated positions being determined by alignment with SEQ ID NO: 1.

In some embodiments of the engineered PolD according to the invention, the C-terminal deletion of DP2 is at least from positions 1220 to 1270; preferably from any one of positions 1191 to 1220 to position 1270, more preferably from positions 1194 to 1270, 1195 to 1270 or 1217 to 1270, the indicated positions being determined by alignment with SEQ ID NO: 2.

In some embodiments of the engineered PolD according to the invention, the truncated subunits are from DP1 and DP2 of a Thermococcales archaea; preferably chosen from Pyrococcus abyssi, Pyrococcus furiosus, Thermococcus nautili, Thermococcus kodakarensis, Thermococcus barophilus and Palaeococcus ferrophilus or a functional variant thereof; more preferably Pyrococcus abyssi or a functional variant thereof. Preferably, the truncated subunits are from DP1 of any one of SEQ ID NO: 1 and 3 to 7 and DP2 of any one of SEQ ID NO: 2 and 8 to 12 or a functional variant thereof.

In some embodiments of the engineered PolD according to the invention, the truncated DP1 subunit comprises a truncated DP1 amino acid sequence having at least 70% identity with the sequence from any one of positions 68 to 197 to position 619 of SEQ ID NO: 1; preferably having at least 70% identity with the sequence from positions 145 to 619 or 197 to 619 of SEQ ID NO: 1.

In some embodiments of the engineered PolD according to the invention, the truncated DP2 subunit comprises a truncated DP2 amino acid sequence having at least 70% identity with the sequence from position 1 to any one of positions 1190 to 1219 of SEQ ID NO: 2; preferably having at least 70% identity with the sequence from positions 1 to 1193, 1 to 1194 or 1 to 1216 of SEQ ID NO: 2.

In some embodiments, the engineered PolD according to the invention is an exonuclease deficient variant; preferably comprising a DP1 subunit having at least one mutation which inactivates PolD exonuclease activity situated at any one of positions D360, H362, D404, Y412, N450, H451, H497, K536, H560, H562, F586 and V590, the indicated positions being determined by alignment with SEQ ID NO: 1; more preferably comprising a DP1 variant chosen from: H451A; D360A and H362A; or N450A, H560A and H562A.

In some embodiments of the engineered PolD according to the invention, the truncated DP1 or DP2 subunit further comprises a tag at the N- or C-terminus; preferably the truncated DP1 comprises a polyhistidine tag at the N-terminus; more preferably a tag 5 comprising the sequence SEQ ID NO: 26.

Another aspect of the invention relates to an expression vector for the recombinant production of an engineered PolD according to any one of claims 1 to 9 in a host cell, comprising a nucleic acid encoding said engineered PolD; preferably comprising the pair of sequences SEQ ID NO: 23 and 25 or SEQ ID NO: 24 and 25.

Another aspect of the invention relates to a method for amplifying a nucleic acid comprising incubating the engineered PolD of according to the present disclosure with a nucleic acid template, at least one oligonucleotide primer and nucleotides under conditions that allow amplification of the nucleic acid template. In some embodiments of the method according to the invention, the amplification is polymerase chain reaction (PCR). In some embodiments of the method according to the invention, the engineered PolD is at a concentration of up to 1 mg/mL; in particular wherein the concentration of the engineered PolD is up to 50 times higher than the maximum effective concentration of wild-type PolD used in the same conditions.

The present invention also encompasses a kit for nucleic acid amplification, in particular polymerase chain reaction (PCR), comprising a least an engineered PolD according to the present disclosure, and optionally, nucleotides, reaction buffer, and/or oligonucleotide primer(s).

The invention relates also to a method for reverse transcription (RT), comprising incubating a polymerase of the family D (PolD) or a functional variant thereof with an RNA template, an oligonucleotide primer and nucleotides under conditions that allow reverse transcription of the RNA template, thereby obtaining a cDNA. In some embodiments, the method of the invention is a method for reverse transcription (RT) and polymerase chain reaction (PCR), further comprising amplifying the obtained cDNA by PCR using said PolD or functional variant thereof. In some embodiments of the method of the invention, the PolD is a thermostable PolD of a hyperthermophilic Thermococcales archaea, in particular chosen from Pyrococcus abyssi, Pyrococcus furiosus, Thermococcus nautili, Thermococcus kodakarensis, Thermococcus barophilus and Palaeococcus ferrophilus or a variant thereof; more particularly Pyrococcus abyssi or a variant thereof. In some embodiments of the method of the invention, the PolD is an engineered PolD according to the present disclosure. In some embodiments of the method of the invention, the PolD is exonuclease deficient, preferably comprising a DP1 subunit having at least one mutation which inactivates PolD exonuclease activity situated at any one of positions D360, H362, D404, Y412, N450, H451, H497, K536, H560, H562, F586 and V590, the indicated positions being determined by alignment with SEQ ID NO: 1; more preferably comprising a DP1 variant chosen from: H451A; D360A and H362A; or N450A, H560A and H562A.

Another aspect of the invention relates to a kit for reverse transcription (RT) or for reverse transcription and polymerase chain reaction (RT-PCR) comprising a polymerase of the family D (PolD) or a functional variant as defined in the present disclosure, wherein the kit does not comprise a reverse transcriptase.

### DETAILED DESCRIPTION OF THE INVENTION

The invention relates to an engineered DNA polymerase of the family D (PolD) and its use for nucleic acid amplification including reverse transcription of RNA.

### Engineered DNA Polymerase PolD

In some embodiments, the invention provides an engineered DNA polymerase of the family D (PolD) comprising: (i) a truncated subunit DP1 comprising a deletion of at least the N-terminal helix-strand-helix (HSH or helix-span-helix) domain and (ii) a truncated subunit DP2 comprising a C-terminal deletion of at least 50 amino acids.

The engineered DNA polymerase D or PolD according to the invention is also named herein PolD-catalytic-core or PolD-catalytic-core construct. The engineered PolD has the following properties compared to the full-length (wild-type) PolD. It is expressed readily in E. coli and is a fully active DNA polymerase as compared to wild-type PolD. It remains active in a wider range of PCR conditions and can therefore be used for a wider range of PCR applications than wild-type PolD. In particular, at higher concentrations of polymerase, the engineered PolD remains active while the activity of wild-type PolD is inhibited. Unexpectedly, PolD, either wild-type PolD or engineered PolD is capable of reverse-transcriptase activity, meaning that it is capable of polymerizing DNA by using RNA as a template. Furthermore, PolD exonuclease-deficient variants show a more efficient reverse-transcriptase activity than the wild-type.

DNA polymerase D (PolD) is the representative member of the D family of DNA polymerases. PolD is a heterodimer composed of a large catalytic subunit (DP2) with 5′-3′ DNA polymerase activity and a smaller subunit (DP1) with 3′-5′ proofreading exonuclease activity. PolD exist in all Archaea except Crenarchea. Representative examples are shown in FIGS. 1 and 2 and include without limitation PolD of Pyrococcus abyssi (DP1 of SEQ ID NO: 1; DP2 of SEQ ID NO: 2); Thermococcus nautili (DP1 of SEQ ID NO: 3; DP2 of SEQ ID NO: 8); Thermococcus kodakarensis (DP1 of SEQ ID NO: 4; DP2 of SEQ ID NO: 9); Palaeococcus ferrophilus (DP1 of SEQ ID NO: 5; DP2 of SEQ ID NO: 10); Thermococcus barophilus (DP1 of SEQ ID NO: 6; DP2 of SEQ ID NO: 11), and Pyrococcus furiosus (DP1 of SEQ ID NO: 7; DP2 of SEQ ID NO: 12).

In the following description, the residues are designated by the standard one letter amino acid code and the indicated positions are determined by alignment with SEQ ID NO: 1 for DP1 or SEQ ID NO: 2 for DP2. One skilled in the art can easily determine the positions in another PolD, by alignment with the reference sequence using appropriate software available in the art such as BLAST, CLUSTALW and others.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

As used herein a C-terminal or N-terminal deletion of a domain, refers to the deletion of consecutive amino acids starting from the N-terminal amino acid (N-terminal deletion) or the C-terminal amino acid (C-terminal deletion).

The N-terminal helix-strand-helix (HSH or helix-span-helix) domain correspond to the sequence from positions 1 to 67 of SEQ ID NO: 1 and the linker domain (or flexible-linker domain) correspond to the sequence from positions 68 to 196 of SEQ ID NO: 1 (FIG. 1). The end of the HSH domain and the start of the linker domain may vary from the indicated positions 67 and 68 by one amino acid (positions 66 and 67) depending on the model used (FIG. 7).

In some embodiments, the truncated subunit DP1 comprises a deletion of the N-terminal helix-strand-helix (HSH) domain. In some embodiments, the truncated subunit DP1 comprises a deletion of the N-terminal helix-strand-helix (HSH) domain and part of the linker domain. In some embodiments, the truncated subunit DP1 comprises a deletion of the N-terminal helix-strand-helix (HSH) domain and all the linker domain. In some embodiments, the deletion is at least from positions 1 to 67 of SEQ ID NO: 1; preferably from position 1 to any one of positions 67 to 196; more preferably from positions 1 to 144 or 1 to 196 of SEQ ID NO: 1.

The C-terminal replication factor interacting domain corresponds to the sequence from positions 1194 to 1270 in SEQ ID NO: 2 (FIG. 2). The start of the C-terminal replication factor interacting domain may vary from the above-indicated position 1194 by one amino acid (position 1195) depending on the model used (FIG. 7). It consists of a basic tail comprising a proliferation cell nuclear antigen (PCNA) interacting domain from positions 1254 to 1265 and a DNA primase interacting domain. In some embodiments, the truncated subunit DP2 comprises a deletion of at least the last 50 amino acids of the C-terminal replicating factor interacting domain. In some embodiments, the truncated subunit DP2 comprises a deletion of all the C-terminal replicating factor interacting domain. In some embodiments, the deletion is at least from positions 1220 to 1270 of SEQ ID NO: 2; preferably from any one of positions 1191 to 1220 to position 1270 of SEQ ID NO: 2; from any one of positions 1194 to 1220 to position 1270 of SEQ ID NO: 2; or from any one of positions 1195 to 1220 to position 1270 of SEQ ID NO: 2; more preferably from positions 1194 to 1270, 1195 to 1270 or 1217 to 1270 of SEQ ID NO: 2.

The engineered PolD according to the invention may be derived from PolD of any Euryarchaeota. In some embodiments, the engineered PolD according to the invention is derived from a thermostable PolD of a hyperthermophilic Thermococcales archaea or a variant thereof. The order Thermococcales includes Pyrococcus, Thermococcus, and Palaeococcus species. In particular embodiments, the engineered PolD is derived from PolD of a Thermococcales archaea chosen from Pyrococcus abyssi, Pyrococcus furiosus, Thermococcus nautili, Thermococcus kodakarensis, Thermococcus barophilus and Palaeococcus ferrophilus or a variant thereof; particularly, Pyrococcus abyssi or a variant thereof. The engineered PolD may be derived from DP1 of any one of SEQ ID NO: 1 and 3 to 7 and DP2 of any one of SEQ ID NO: 2 and 8 to 12 or a variant thereof.

The boundaries of the DP1 N-terminal HSH and linker domains and DP2 C-terminal replication factor interacting-domain have been determined by generating 3D models for each PolD homolog using AlphaFold2 (Mirdita et al., Nature Methods, 19, June 2022, 679-682) as illustrated in FIG. 7. The boundaries for the DP1 HSH and linker domains determined using this model are the following: Pyrococcus furiosus of SEQ ID NO: 7 (HSH 1-68; linker-domain 69-190), Thermococcus barophilus of SEQ ID NO: 6 (HSH 1-65; linker-domain 66-253), Thermococcus kodakarensis of SEQ ID NO: 4 (HSH 1-62; linker-domain 63-310), Thermococcus nautili of SEQ ID NO: 3 (HSH 1-62, linker-domain 63-300) and Paleococcus ferrophilus of SEQ ID NO: 5 (HSH 1-61, linker-domain 62-217). The boundaries for the DP2 C-terminal replication factor interacting-domain determined using this model are the following: Pyrococcus furiosus of SEQ ID NO: 12 (1193-1263), Thermococcus barophilus of SEQ ID NO: 11 (1188-1281), Thermococcus kodakarensis of SEQ ID NO: 9 (1203-1324), Thermococcus nautili of SEQ ID NO: 8 (1197-1291) and Paleococcus ferrophilus of SEQ ID NO: 10 (1182-1262).

As used herein, the term “variant” refers to a polypeptide comprising an amino acid sequence having at least 70% sequence identity with the native sequence. The term “variant” refers to a functional variant having the activity of the native sequence. Functional fragments of the native sequence or variant thereof are also encompassed by the present disclosure. The activity of a variant or fragment may be assessed using methods well-known by the skilled person such as those disclosed herein.

As used herein, the term “functional variant”, refers to a DP1 or DP2 variant that forms a functional heterodimer having DNA polymerase activity in PCR reaction (PCR activity). PCR activity may be assayed using standard assay, in the presence of a nucleic acid template, a pair of complementary forward and reverse oligonucleotide primers, nucleotides, and an appropriate reaction buffer as known in the art (Killelea et al., Front. Microbiol., 2014, 5, 195) and disclosed in the examples of the present application.

The truncated DP1 comprises or consists of a N-terminally truncated DP1 amino acid sequence. In some embodiments, the truncated DP1 amino acid sequence consists of the sequence from any one of positions 68 to 197 to position 619 of SEQ ID NO: 1 or a variant thereof, preferably from positions 145 to 619 or 197 to 619 of SEQ ID NO: 1 or a variant thereof. For example, the N-terminally truncated DP1 amino acid sequence derived from Pyrococcus abyssi consists of 423 to 552 amino acids, preferably 475 amino acids. In some embodiments, the truncated DP1 subunit comprises a N-terminally truncated DP1 amino acid sequence having at least 70%, 75%, 80% or more identity with the sequence from any one of positions 68 to 197 to position 619 of SEQ ID NO: 1; preferably at least 85%, 86%, 87%, 88%, 89%, 90%, 91%, 92%, 93%, 94%, 95%, 96%, 97%, 98%, 99% or 100% identity with the sequence from any one of positions 68 to 197 to position 619 of SEQ ID NO: 1. In some particular embodiments, the truncated DP1 subunit comprises a N-terminally truncated DP1 amino acid sequence having at least 70%, 75%, 80% or more identity with the sequence from position 145 to position 619 or from position 197 to position 619 of SEQ ID NO: 1; preferably at least 85%, 86%, 87%, 88%, 89%, 90%, 91%, 92%, 93%, 94%, 95%, 96%, 97%, 98%, 99% or 100% identity with the sequence from position 145 to position 619 or from position 197 to position 619 of SEQ ID NO: 1. In some preferred embodiments, the truncated DP1 is selected from the group consisting of the sequences SEQ ID NO: 13, 14, 18 or 19.

The truncated DP2 comprises or consists of a C-terminally truncated DP2 amino acid sequence. In some embodiments, the truncated DP2 amino acid sequence consists of the sequence from position 1 to any one of positions 1190 to 1219 of SEQ ID NO: 2 or a variant thereof, from position 1 to any one of positions 1193 to 1219 of SEQ ID NO: 2 or a variant thereof, or from position 1 to any one of positions 1194 to 1219 of SEQ ID NO: 2 or a variant thereof; preferably from positions 1 to 1193, 1 to 1194 or 1 to 1216 of SEQ ID NO: 2 or a variant thereof. For example, the C-terminally truncated DP1 amino acid sequence derived from Pyrococcus abyssi consists of 1190 to 1219 amino acids, preferably 1193, 1194 or 1216 amino acids. In some embodiments, the truncated DP2 subunit comprises a C-terminally truncated DP2 amino acid sequence having at least 70%, 75%, 80% or more identity with the sequence from position 1 to any one of positions 1190 to 1219 of SEQ ID NO: 2; preferably at least 85%, 86%, 87%, 88%, 89%, 90%, 91%, 92%, 93%, 94%, 95%, 96%, 97%, 98%, 99% or 100% identity with the sequence from position 1 to any one of positions 1190 to 1219 of SEQ ID NO: 2. In some embodiments, the truncated DP2 subunit comprises a C-terminally truncated DP2 amino acid sequence having at least 70%, 75%, 80% or more identity with the sequence from position 1 to any one of positions 1193 or 1194 to 1219 of SEQ ID NO: 2; preferably at least 85%, 86%, 87%, 88%, 89%, 90%, 91%, 92%, 93%, 94%, 95%, 96%, 97%, 98%, 99% or 100% identity with the sequence from position 1 to any one of positions 1193 or 1194 to 1219 of SEQ ID NO: 2. In some particular embodiments, the truncated DP2 subunit comprises a C-terminally truncated DP2 amino acid sequence having at least 70%, 75%, 80% or more identity with the sequence from position 1 to position 1193, from position 1 to position 1194 or from position 1 to position 1216 of SEQ ID NO: 2; preferably at least 85%, 86%, 87%, 88%, 89%, 90%, 91%, 92%, 93%, 94%, 95%, 96%, 97%, 98%, 99% or 100% identity with the sequence from position 1 to position 1193, from position 1 to position 1194 or from position 1 to position 1216 of SEQ ID NO: 2. In some preferred embodiments, the truncated DP2 is SEQ ID NO: 15.

The percent amino acid sequence or nucleotide sequence identity is defined as the percent of amino acid residues or nucleotides in a Compared Sequence that are identical to the Reference Sequence after aligning the sequences and introducing gaps if necessary, to achieve the maximum sequence identity and not considering any conservative substitutions for amino acid sequences as part of the sequence identity. Alignment for purposes of determining percent amino acid sequence identity can be achieved in various ways known to a person of skill in the art, for instance using publicly available computer software such as the GCG (Genetics Computer Group, Program Manual for the GCG Package, Version 7, Madison, Wisconsin) pileup program, or any of sequence comparison algorithms such as BLAST (Altschul et al., J. Mol. Biol., 1990, 215, 403-), FASTA or CLUSTALW. When using such software, the default parameters, are preferably used.

In some embodiments, the term “variant” refers to a polypeptide having an amino acid sequence that differs from a native sequence by the substitution, insertion and/or deletion of less than 100, 90, 80, 70, 60, 50, 40, 30, 25, 20, 15, 10 or 5 amino acids. In a preferred embodiment, the variant differs from the native sequence by one or more conservative substitutions, preferably by less than 50, 40, 30, 25, 20, 15, 10 or 5 conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine), and small amino acids (glycine, alanine, serine and threonine).

In some embodiments, the engineered PolD is exonuclease deficient. Exonuclease deficient PolD variant comprises at least one mutation that inactivates the nuclease proof-reading activity of DP1 subunit. These mutations are located in the nuclease active site of DP1 (FIG. 3), preferably at any one of positions D360, H362, D404, Y412, N450, H451, H497, K536, H560, H562 F586 and V590. Exonuclease deficient PolD variant may be identified by standard 3′-5′ exonuclease assay that are well-known in the art (Sauguet et al., 2016, precited). In some embodiments, the mutation is a substitution, in particular by a different amino acid such as alanine or other amino acid. In particular embodiments the substitution is an alanine substitution. In some preferred embodiments, the DP1 variant is chosen from H451A; D360A and H362A; or N450A, H560A and H562A. In particular, the DP1 variant H451A may be chosen from the sequences SEQ ID NO: 14 and 19.

The truncated DP1 and DP2 may further comprise a heterologous sequence, which means a sequence different from the sequence naturally present in the native DP1 and DP2 sequence. The heterologous sequence is usually of up to 50 amino acids. The heterologous sequence may be added at the N-terminus and/or C-terminus of the truncated DP1 or DP2 sequence. The truncated DP1 comprises a N-terminal methionine for translation initiation. In some embodiments, the heterologous sequence is added at the N-terminus of the truncated DP1 sequence. In some embodiments, the added heterologous sequence is a tag, in particular a purification tag suitable for affinity purification such as polyhistidine tag or streptavidine tag. Polyhistidine tag usually comprises at least 5 histidines which bind to metal matrices comprising nickel or cobalt. The tag may be removable by chemical agents or by enzymatic means such as proteases (TEV protease, Thrombin, Factor Xa or Enteropeptidase). In some particular embodiments, the tag comprises or consists of the sequence: MGKHHHHSGHHHTGHHHHSGSHHHTSSSASTGENLYFQGTGDGS (SEQ ID NO: 26); the polyhistidine tag is removable by TEV protease which recognizes the cleavage site ENLYFQG (SEQ ID NO: 27).

### Nucleic Acid, Vector, Cell

The invention relates also to an isolated nucleic acid comprising a nucleotide sequence encoding the engineered DNA polymerase PolD in expressible form; preferably comprising nucleotide sequences encoding the truncated DP1 and DP2 subunits.

The nucleic acid encoding the engineered PolD in expressible form refers to a nucleic acid molecule which, upon expression in a cell or a cell-free system, results in a functional protein.

The nucleic acid may be recombinant, synthetic or semi-synthetic nucleic acid which is expressible in the recombinant cell. The nucleic acid may be DNA, RNA, or mixed molecule, either single- and/or double-stranded which may further be modified and/or included in any suitable expression vector. The nucleic acid may comprise a coding sequence which is optimized for the host in which the PolD construct is expressed.

In some embodiments said nucleic acid comprises at least a sequence selected from the group consisting of: SEQ ID NO: 23 to 25.

The coding sequence is operably linked to appropriate regulatory sequence(s) for its expression in the host cell (recombinant cell). Such sequences which are well-known in the art include in particular a promoter, and further regulatory sequences capable of further controlling the expression of a transgene, such as without limitation, enhancer or activator, terminator, kozak sequence and intron (in eukaryote), ribosome-binding site (RBS) (in prokaryote). In some particular embodiments, the coding sequence is operably linked to a promoter. The promoter may be a ubiquitous, constitutive or inducible promoter that is functional in the recombinant cell.

As used herein, the terms “vector” and “expression vector” mean the vehicle by which a DNA or RNA sequence (e.g. a foreign gene) can be introduced and maintained into a host cell, so as to transform the host and promote expression (e.g. transcription and translation) of the introduced sequence. The recombinant vector can be a vector for eukaryotic or prokaryotic expression, such as a plasmid, a phage for bacterium introduction, a YAC able to transform yeast, a transposon, a mini-circle, a viral vector, or any other expression vector. The vector may be a replicating vector such as a replicating plasmid. The replicating vector such as replicating plasmid may be a low-copy or high-copy number vector or plasmid.

Another aspect of the invention relates to an expression vector for the recombinant production of an engineered PolD according to the present disclosure in a host cell, comprising a nucleic acid encoding said engineered PolD according to the present disclosure.

In some particular embodiments, the expression vector according to the present disclosure comprises a pair of nucleic acid sequences selected from: a sequence having at least 90% identity with SEQ ID NO: 23 and a sequence having at least 90% identity with SEQ ID NO: 25; a sequence having at least 90% identity with SEQ ID NO: 24 and a sequence having at least 90% identity with SEQ ID NO: 25. In some embodiments, the nucleic acid sequence is DNA. In some particular embodiments, the expression vector is a prokaryote expression vector, particularly a plasmid.

The nucleic acid according to the invention is prepared by the conventional methods known in the art. For example, it is produced by amplification of a nucleic sequence by PCR or RT-PCR, by screening genomic DNA libraries by hybridization with a homologous probe, or else by total or partial chemical synthesis. The recombinant vectors are constructed and introduced into host cells by the conventional recombinant DNA techniques, which are known in the art.

A further aspect of the invention provides a host cell comprising the nucleic acid or recombinant vector. Prokaryote cell is in particular bacteria. In some embodiments, the prokaryotic cell is a bacterial cell, in particular an E. coli cell.

Another aspect of the invention relates to a method of production of the engineered PolD according to the present disclosure, comprising: (i) culturing the host cell of the present disclosure for expression of said engineered PolD by the host cell; (ii) recovering the engineered PolD from the culture medium or host cells; and (iii) purifying said engineered PolD.

### Use of Engineered PolD for Nucleic Acid Amplification

The invention also encompasses the use of the engineered DNA polymerase PolD according to the present disclosure for nucleic acid amplification, as well as methods of using the same and kits thereof.

In one embodiment, the invention relates to a method for amplifying a nucleic acid comprising incubating the engineered PolD of the invention with a nucleic acid template, at least one oligonucleotide primer and nucleotides under conditions that allow amplification of the nucleic acid template. Such conditions are well-known in the art (Killelea et al., Front. Microbiol., 2014, 5, 195) and disclosed in the examples of the present application.

The nucleic acid template is any target nucleic acid of interest. The nucleic acid template may be DNA or mixed nucleic acid. The nucleic acid template, oligonucleotide primers and nucleotides may comprise natural deoxy-ribonucleotides (dATP, dGTP, dCTP, dTTP, dUTP), modified deoxy-ribonucleotides or any combination of natural deoxy-ribonucleotides and modified deoxy-ribonucleotides, in addition they may include some natural ribonucleotides (ATP, GTP, CTP, UTP) or modified ribonucleotides. The oligonucleotide primer(s) hybridizes to the 3′-end(s) of the nucleic acid.

In particular embodiments, said nucleic acid amplification is polymerase chain reaction (PCR). PCR uses a pair (forward and reverse) of oligonucleotide primers. PCR uses a thermocycler to perform cycles of a denaturation step, a primer annealing step and an elongation step. Exemplary conditions are set forth in the examples. In various embodiments, the time for the elongation step is 1 min/kb or less.

In particular embodiments, the engineered PolD is at a concentration of up to 1000 μg/mL, in particular from 4 μg/mL to 400 μg/mL, more particularly 4, 10, 20, 40, 100, 200, 400 μg/mL. In particular embodiments, the engineered PolD is at a concentration which is at least 2 times higher, preferably at least 5, 10, 20 or 50 times or more higher than the maximum effective concentration of wild-type PolD used in the same conditions.

The present invention also encompasses a kit for nucleic acid amplification, preferably by PCR, comprising a least an engineered PolD according to the present disclosure, and optionally, nucleotides, reaction buffer, and/or oligonucleotide primer(s).

The engineered PolD may be used in a wide variety of protocols and technologies which use PCR and has numerous applications, in particular in research and diagnostics.

### Use of PolD for Reverse Transcription

The invention also encompasses the use of PolD for reverse transcription, as well as methods of using the same and kits thereof.

In one embodiment, the invention relates to a method for reverse transcription (RT), comprising incubating a polymerase of the family D (PolD) or a functional variant thereof with an RNA template, an oligonucleotide primer and nucleotides under conditions that allow reverse transcription of the RNA template. Exemplary conditions are disclosed in the Examples. The reverse transcription may be performed at a temperature of about 55° C. to about 72° C.; preferably about 72° C. The buffer is the usual buffer used for PCR reaction. The PolD is at an appropriate concentration for reverse transcription, in particular about 200 μg/mL.

The RNA template is any target nucleic acid of interest. The nucleic acid template may comprise natural ribonucleotides (ATP, GTP, CTP, UTP), modified ribonucleotides or mixture thereof. The oligonucleotide primers and nucleotides may comprise natural deoxy-ribonucleotides (dATP, dGTP, dCTP, dTTP, dUTP), modified nucleotides or any combination of natural deoxy-ribonucleotides and modified nucleotides.

In one embodiment, the invention relates to a method for reverse transcription (RT) and polymerase chain reaction (PCR), comprising: a) incubating a polymerase of the family D (PolD) or a functional variant thereof with an RNA template, an oligonucleotide primer and nucleotides under conditions that allow reverse transcription of the RNA template, thereby obtaining a cDNA and b) amplifying the obtained cDNA by PCR using said PolD or functional variant thereof.

Conditions to perform PCR with PolD are well-known in the art (Killelea et al., Front. Microbiol., 2014, 5, 195) and disclosed in the examples of the present application. PCR reaction is performed in the presence of a pair of primers (forward and reverse primer), nucleotides and suitable buffer. The reverse primer may be the same as the primer for the reverse transcription or a different primer.

The PolD may PolD of any Euryarchaeota or a functional variant thereof. In some embodiments, the PolD is a thermostable PolD of a hyperthermophilic Thermococcales archaea, in particular chosen from Pyrococcus abyssi, Pyrococcus furiosus, Thermococcus nautili, Thermococcus kodakarensis, Thermococcus barophilus and Palaeococcus ferrophilus or a variant thereof; more particularly Pyrococcus abyssi or a variant thereof. In some embodiments the PolD comprises DP1 and DP2 chosen from: SEQ ID NO: 1 and 2; SEQ ID NO: 3 and 8; SEQ ID NO: 4 and 9; SEQ ID NO: 5 and 10; SEQ ID NO: 6 and 11; SEQ ID NO: 7 and 12. of any one of SEQ ID NO: 1 and 3 to 7 and DP2 of any one of SEQ ID NO: 2 and 8 to 12 or a variant thereof.

In some embodiments, the PolD is an engineered PolD according to the present disclosure.

In some embodiments, the PolD is exonuclease deficient. Exonuclease deficient PolD have an increased reverse transcriptase activity compared to wild-type PolD. Exonuclease deficient PolD variant comprises at least one mutation that inactivates the nuclease proof-reading activity of DP1 subunit. These mutations are located in the nuclease active site of DP1 (FIG. 3), preferably at any one of positions D360, H362, D404, Y412, N450, H451, H497, K536, H560, H562, F586 and V590. Exonuclease deficient PolD variant may be identified by standard 3′-5′ exonuclease assay that are well-known in the art (Sauguet et al., 2016, precited). In some embodiments, the mutation is a substitution, in particular by a different amino acid such as alanine or other amino acid. In particular embodiments, the substitution is an alanine substitution. In more particular embodiments, the substitution(s) is chosen from H451A; D360A and H362A; or N450A, H560A and H562A. In particular, the DP1 variant H451A may be chosen from the sequences SEQ ID NO: 14, 17 and 19.

The present invention also encompasses a kit for reverse transcription (RT), comprising a polymerase of the family D (PolD) or a functional variant thereof according to the present disclosure, and optionally, nucleotides, reaction buffer, and/or oligonucleotide primer. The kit does not comprise a reverse transcriptase. In some particular embodiments, the kit comprises an engineered PolD according to the present disclosure.

In some embodiments, the kit is for reverse transcription and polymerase chain reaction (PCR); optionally further comprising a forward primer.

The practice of the present invention will employ, unless otherwise indicated, conventional techniques which are within the skill of the art. Such techniques are explained fully in the literature.

The invention will now be exemplified with the following examples, which are not limitative, with reference to the attached drawings in which:

### FIGURE LEGENDS

FIG. 1: Multiple-sequence alignment showing the conservation of the DP1 subunit in a representative set of Thermococalles archaea: Pyrococcus abyssi (SEQ ID NO: 1), Thermococcus nautili (SEQ ID NO: 3), Thermococcus kodakarensis (SEQ ID NO: 4), Palaeococcus ferrophilus (SEQ ID NO: 5), Thermococcus barophilus (SEQ ID NO: 6), and Pyrococcus furiosus (SEQ ID NO: 7).

FIG. 2: Multiple-sequence alignment showing the conservation of the DP2 subunit in a representative set of Thermococalles archaea: Pyrococcus abyssi (SEQ ID NO: 2), Thermococcus nautili (SEQ ID NO: 8), Thermococcus kodakarensis (SEQ ID NO: 9), Palaeococcus ferrophilus (SEQ ID NO: 10), Thermococcus barophilus (SEQ ID NO: 11), and Pyrococcus furiosus (SEQ ID NO: 12).

FIG. 3: Active site residues important for the nuclease activity of DP1 from Sauguet et al., Nature communications, 2016, 7, 12227.

FIG. 4: The four PolD constructs are able to perform PCR on a 2.6 kb-long amplicon at a concentration of 20 μg/mL and with 1 min/kb of elongation time in the cycling conditions.

FIG. 5: PCR activities of PolD-exo- and PolD-catalytic-core-exo-mut1 at different concentrations of 4 μg/mL, 10 μg/mL, 20 μg/mL, 40 μg/mL, 100 μg/mL, 200 μg/mL 400 μg/mL and 1000 μg/mL.

FIG. 6: Reverse transcriptase activities of PolD constructs (PolD wild-type, PolD-exo-(mut1, mut2 and mut3), PolD-catalytic-core-exo-(mut1, mu2 and mut3). Reaction was performed at 72° C. with different templates and a fluorescence-labeled DNA primer for different incubation times (in min) as indicated.

FIG. 7: 3D models for each PolD homolog using AlphaFold2 showing the boundaries of the DP1 N-ter HSH and linker domains and DP2 C-ter replication factor interacting domain.

### EXAMPLES

### Example 1: Design, Expression and Purification of Engineered PolD Constructs

1. Identification and Deletion of Domains which are not Mandatory for Enzymatic Activity

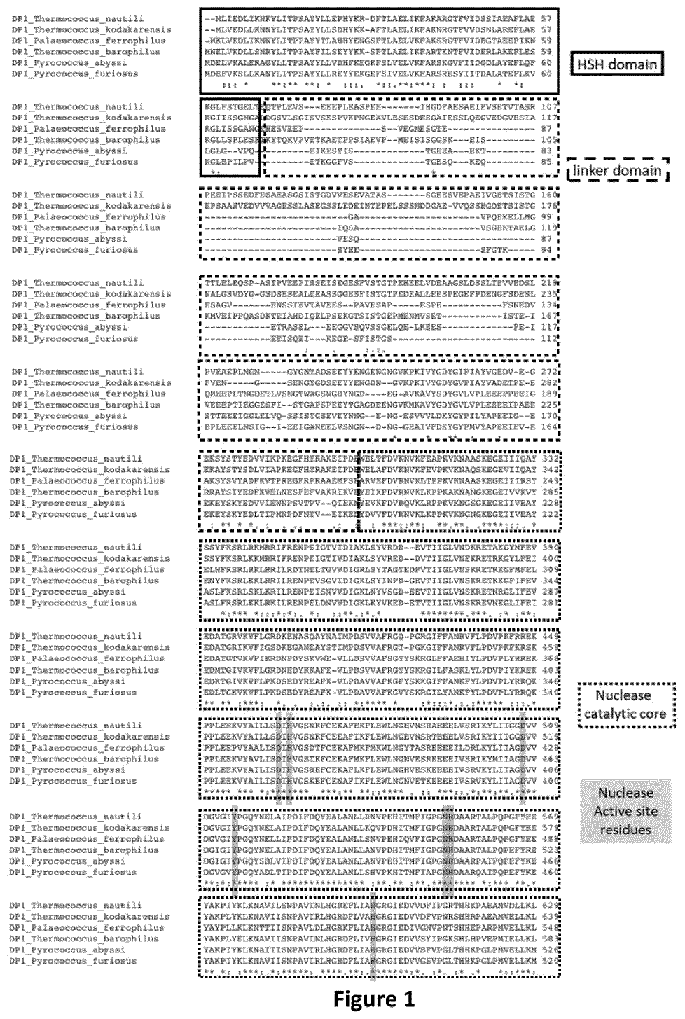

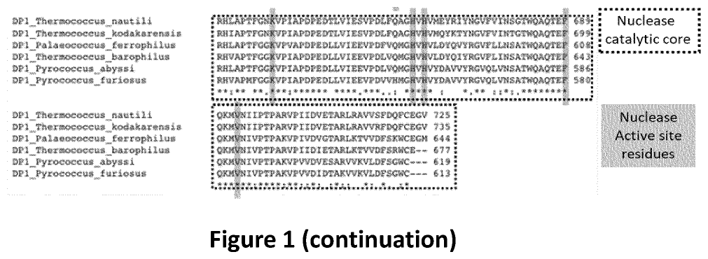

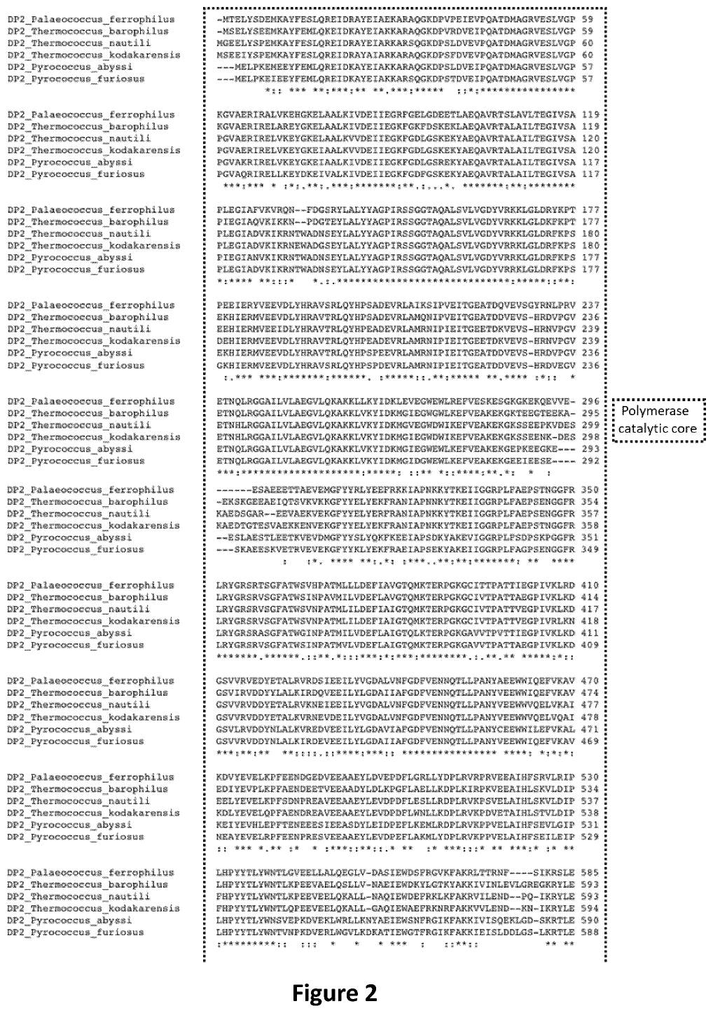

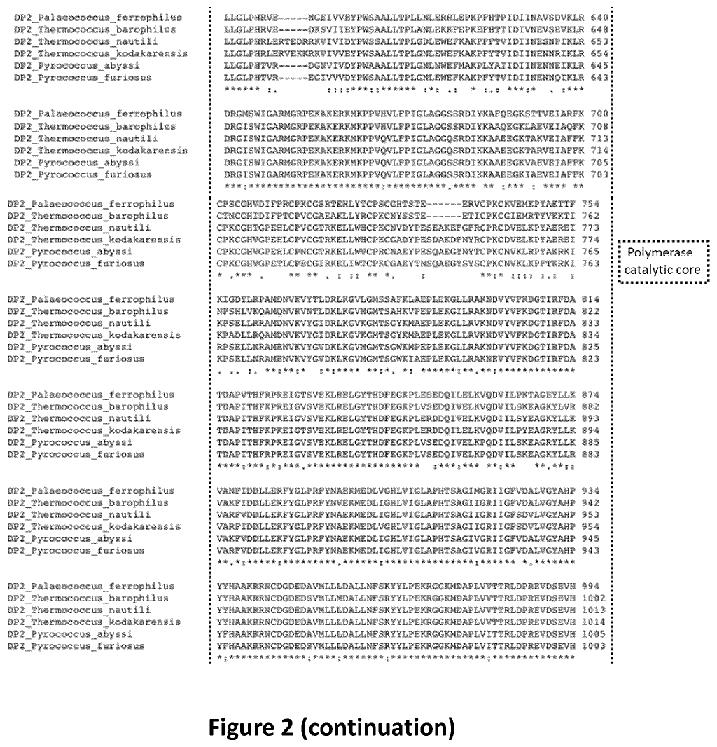

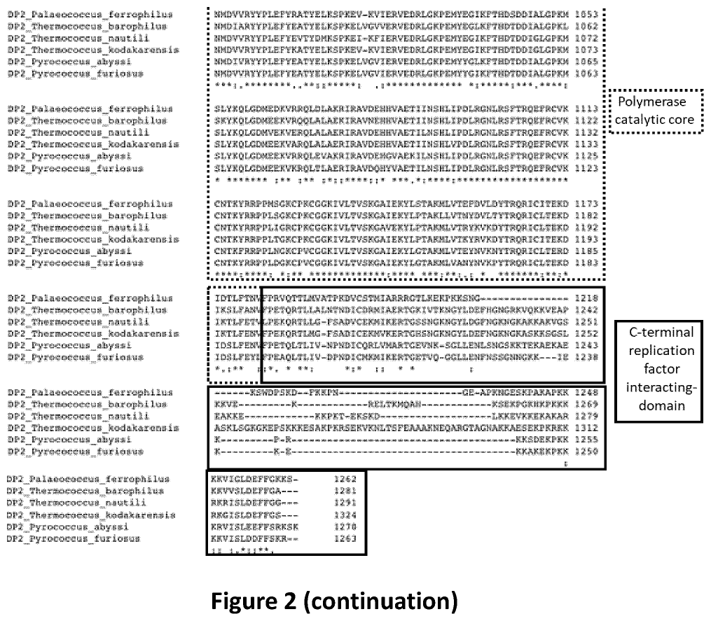

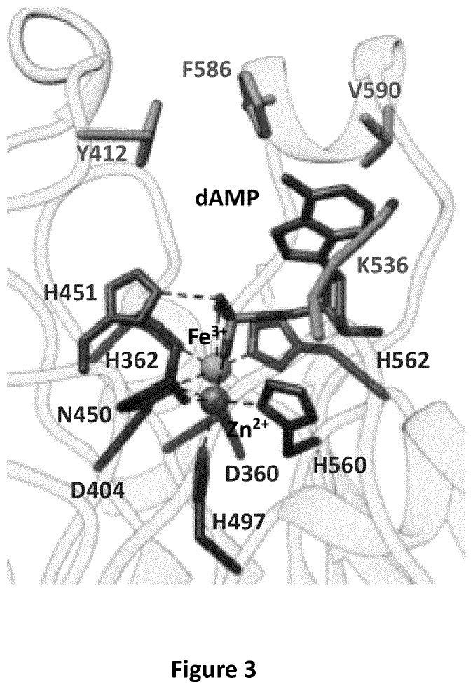

Based on the structures of PolD that were solved previously (Sauguet et al., Nature communications, 2016, 7, 12227; Raia et al., PLOS Biology, 2019, 18, 17 (1) e3000122; Madru et al., Nature communications, 2020, 27, 11 (1), 1591), the inventors have identified and deleted two domains, which are non-essential for PolD's catalytic activity. The first domain that was deleted is located in the N-terminal region of the DP1 subunit (FIG. 1). This domain is a HSH (helix-span-helix) domain (positions 1 to 67 of SEQ ID NO: 1) that interacts, in the cell, with other partners of the DNA replication machinery, including the replicative helicase. This domain is connected to the phosphodiesterase domain of DP1 by a flexible linker-domain (positions 68 to 196 of SEQ ID NO: 1) that was also deleted in part. The truncated DP1 subunit thus comprises a N-terminal deletion up to position 144 of DP1 amino acid sequence (DP1-ΔN(1-144) construct). The second domain is located in the C-terminal region of the DP2 subunit (FIG. 2). In the living cell, this domain interacts with other replication factors, including the DNA primase and the Proliferating Cell Nuclear Antigen (PCNA). The truncated DP2 subunit comprises a C-terminal deletion starting from position 1217 of DP2 amino acid sequence (DP2-ΔC(1217-1270) construct). This new construct comprising the truncated DP1 and DP2 subunits was named the PolD-catalytic-core. Three exonuclease deficient (exo-) PolD-catalytic-core were also constructed: PolDexo-mut1, derived from the DP1 variant H451A (DP1-H451A) previously disclosed in Palud et al. (Mol. Microbiol., 2008, 70, 746-761); PolDexo-mut2 comprising the substitutions D360A and H362A and; PolDexo-mut3 comprising the substitutions N450A, H560A and H562A. Other constructs containing a truncated DP1 having a deletion of either only the HSH domain (deletion from positions 1 to 67 of SEQ ID NO: 1) or the HSH domain and all the linker domain (deletion from positions 1 to 196 of SEQ ID NO: 1) were also tested and found able to form a functional polymerase in association with truncated DP2 subunit. However, DP1-ΔN(1-144) construct was found optimal in terms of protein solubility. Other constructs containing a truncated DP2 having a deletion of all the C-terminal replication factor interacting domain (positions 1194 to 1220) were also tested and found able to form a functional polymerase in association with truncated DP1 subunit.

### 2. Cloning of PolD Constructs

Both DP1 and DP2 genes were cloned into a pRSF-Duet™ vector (Novagen), which is designed for the coexpression of two target proteins. The vector encodes two multiple cloning sites (MCS) each of which is preceded by a T7 promoter, lac operator, and ribosome binding site (rbs). The vector also carries the pRSF1030 replicon (also known as NTP1), lacI gene, and kanamycin resistance marker. The DP1 construct contains an N-terminal poly-histidines expression tag and was cloned within the NcoI and NotI cloning sites. The DP2 construct was cloned within the NdeI and XhoI cloning sites. Nine constructs derived from PolD of Pyrococcus abyssi were generated:
1) PolD wild-type: DP1-DP2
   DP1 construct: nucleotide (nt) sequence SEQ ID NO: 20; amino acid (aa) sequence SEQ ID NO: 16; and
   DP2 construct: nt sequence SEQ ID NO: 21; aa sequence SEQ ID NO: 2.
2) PolD-exo-(PolDexo-mut1): DP1-H451A-DP2
   DP1-H451A construct: nt sequence SEQ ID NO:22; aa sequence SEQ ID NO: 17; and
   DP2 construct: nt sequence SEQ ID NO: 21; aa sequence SEQ ID NO: 2.
3) PolDexo-mut2:
   DP1-D360A and H362A construct; and
   DP2 construct: nt sequence SEQ ID NO: 21; aa sequence SEQ ID NO: 2.
4) PolDexo-mut2:
   DP1-D360A and H362A construct; and
   DP2 construct: nt sequence SEQ ID NO: 21; aa sequence SEQ ID NO: 2.
5) PolDexo-mut3:
   DP1-N450A, H560A and H562A construct; and
   DP2 construct: nt sequence SEQ ID NO: 21; aa sequence SEQ ID NO: 2.
6) PolD-catalytic-core: DP1-ΔN(1-144)-DP2-ΔC(1217-1270)
   DP1-ΔN(1-144) construct: nt sequence SEQ ID NO: 23; aa sequence SEQ ID NO: 18; and
   DP2-ΔC(1217-1270) construct: nt sequence SEQ ID NO: 25; aa sequence SEQ ID NO: 15.
7) PolD-catalytic-core-exo-(PolD-catalytic-core-exo-mut1): DP1-ΔN(1-144)-H451A-DP2-ΔC(1217-1270)
   DP1-ΔN(1-144)-H451A construct: nt sequence SEQ ID NO:24; aa sequence SEQ ID NO: 19; and
   DP2-ΔC(1217-1270) construct: nt sequence SEQ ID NO: 25; aa sequence SEQ ID NO: 15.
8) PolD-catalytic-core-exo-mut 2
   DP1-ΔN(1-144)-D360A and H362A construct; and
   DP2-ΔC(1217-1270) construct: nt sequence SEQ ID NO: 25; aa sequence SEQ ID NO: 15.
9) PolD-catalytic-core-exo-mut 3
   DP1-ΔN(1-144)-N450A, H560A and H562A construct; and
   DP2-ΔC(1217-1270) construct: nt sequence SEQ ID NO: 25; aa sequence SEQ ID NO: 15.
      3. Recombinant Expression of PolD in E. coli

### Bacterial Transformation

Two different strains of Escherichia coli competent cells were tested, the BL21-DE3 star (Thermofischer) and KXR (Promega). Competent cells were transformed by using 500 ng of plasmid. The mixture was kept on ice for 15-30 minutes. Cells were heat shocked at 42° C. for 30 seconds, and shaked for one hour at 37° C. in 1 ml SOC medium. Finally, cells were spread on LB-Agar (Lysogeny broth medium) plates+50 ng/ul kanamycine and incubated at 37° C. overnight.

### Bacterial Culture & Recombinant Protein Expression

For each construct, a 100 ml culture of LB+50 ng/ul kanamycine was inoculated using several colonies and incubated at 37° C. overnight, 180 rpm. A fresh culture was then inoculated (starter OD600) and incubated at 37° C., 180 rpm. When its optical density at 600 nm (OD600) reached 0.6, the culture were chilled at 4° C. for 20 minutes. Protein expression was induced by adding 0.5 mM isopropyl-β-D-1-thiogalactopyranoside (IPTG) or 0.1% L-Rhamnose for BL21-DE3 star cells and KRX cells, respectively. After induction, cells were incubated at 20° C., 180 rpm, for 20 hours. Cells were harvested by centrifugation, washed once with fresh LB and stored at −20° C.

### 4. Purification of PolD

### Buffers Used for Purification

The following buffers were used for protein purification:

TABLE 1
Buffers for protein purification

| Buffer | A | B | C | D | E | F |
|---|---|---|---|---|---|---|
| Tris- | 50 mM | 50 mM | 50 mM | 50 mM | 50 mM | 50 mM |
| HCl | | | | | | |
| (pH 8) | | | | | | |
| NaCl | 300 mM | 300 mM | / | 50 mM | 2M | 200 mM |
| Imida- | 10 mM | 500 mM | / | / | / | / |
| zole | | | | | | |
| MgCl2 | / | / | / | / | / | 3 mM |

### Purification Procedure for PolD

Cells were resuspended in Buffer supplemented with complete EDTA-free protease inhibitors (Thermo Fisher) and 500 units of benzonase (Sigma). Resuspended cells were then lysed by mechanical disruption with 3 passes through a pre-cooled cell disruptor (Constant System Limited) at 1.4 kPa, and the lysate was centrifuged at 20 000 g for 30 minutes at 4° C. All the following steps described below were performed with chromatography columns from GE Healthcare connected to an ÄKTA pure system (GE Healthcare) at room temperature. After centrifugation, the clear supernatant containing PolD was loaded onto a 5 mL HisTrap nickel affinity column (GE Healthcare). The column was then washed with 5 column volumes of buffer A. The complex was finally eluted using a 50 mL linear gradient of imidazole (0%-100% HisTrap Buffer B). Fractions were analyzed by SDS-PAGE 4-20%. PolD-containing HisTrap fractions were combined and 5-fold diluted in Buffer C before being loaded onto a 5 ml Heparin column (GE Healthcare), pre-equilibrated in Buffer D. The column was washed with 25 mL of Buffer D while PolD was eluted with a 50 mL linear gradient of NaCl realized by mixing Buffer D with Buffer E. The purest fractions containing PolD complex were dialized against Buffer F. PolD was concentrated up-to 20 mg/mL. 20% glycerol were added to the concentrated PolD before it was flash-frozen in liquid nitrogen and stored at −80° C. Final yield: 3-4 mg of purified and concentrated PolD were obtained from 1 liter of culture.

The nine PolD constructs were readily expressed in different E. coli cell lines and purified to homogeneity.

### Example 2: PCR Amplification Activity

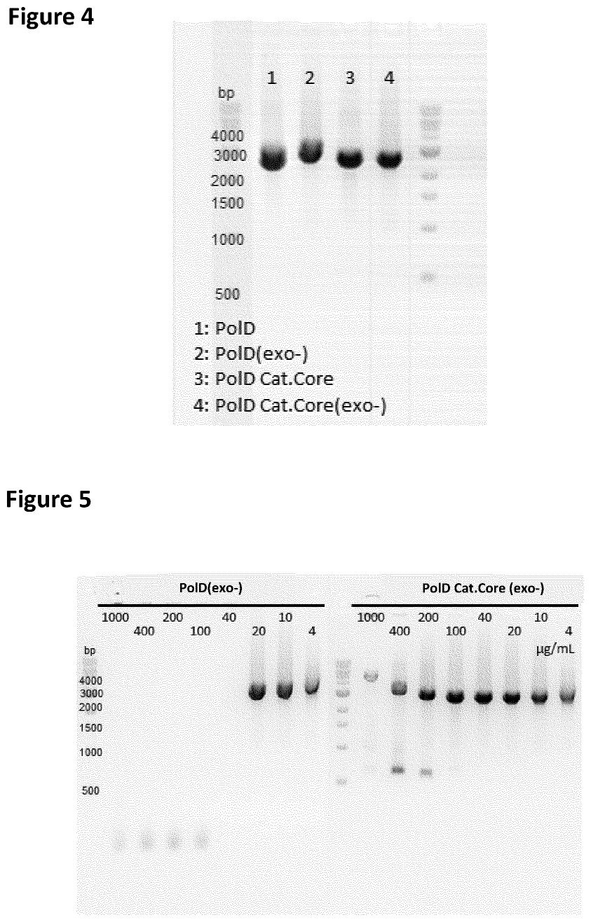

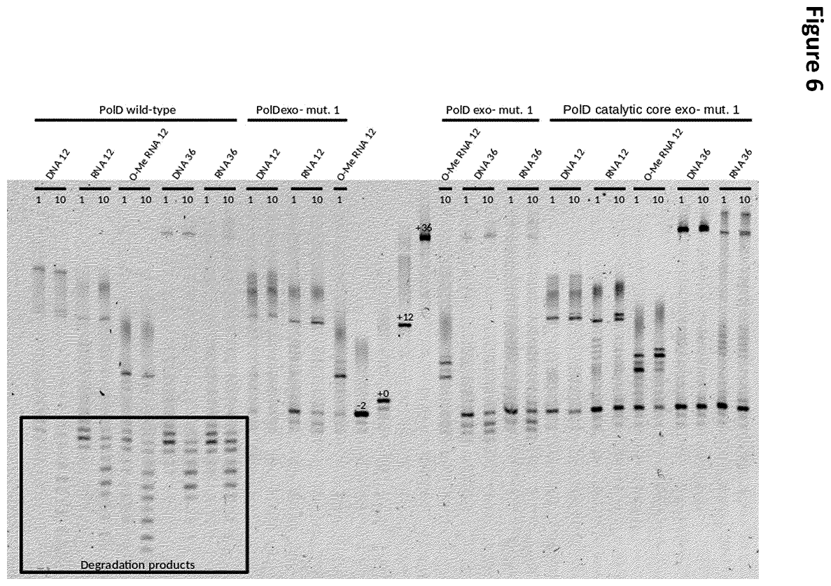

The ability of PolD to perform PCR reactions had only been studied once (Killelea et al., Front. Microbiol., 2014, 5, 195). The found PCR conditions were unusually limiting (small fragments, long elongation time . . . ). The inventors investigated the reaction conditions to obtain PCR of larger fragments in more usual conditions (FIG. 4). The reaction mixture was composed of PolD Reaction Buffer 1× (20 mM TrisHCl, pH9, 25 mM KCl, 10 mM (NH₄)₂SO₄, 2 mM MgCl₂, 1 mg/mL BSA, 0.1% v/v Tween20), 200 μM dNTPs, 200 nM each primer (P2,6 kb_Fwd: ctactactctctttatcagaagttcaaggaggat (SEQ ID NO: 28); P2,6 kb_rev: cgattaaagttaactgggtctctgggaa (SEQ ID NO: 29), DNA target (DP2 gene cloned in plasmid; 2.6 kb amplicon) in the fM to pM range and the polymerase in various concentrations. The mixes were incubated in a thermocycler (94° C., 30 s for denaturation; 55 to 72° C., 30 s for primers annealing; 72° C. 1 to 4 min/kb for elongation; 30 cycles) and the PCR products were analyzed on a 0.8% agarose gel. After several experiments, it was shown that, after purification, the four PolD constructs are able to perform PCR on a 2.6 kb-long amplicon with 1 min/kb of elongation time in the cycling conditions (FIG. 4) instead of 4 min/kb reported once in the literature (Killelea et al., 2014, precited). Furthermore, it was shown that PolD-exo-construct, at a concentration of 200 μg/mL, is capable of performing PCR on a DNA fragment of 4 kb, 4 folds longer than previously reported in the literature (Killelea et al., 2014, precited). Finally, the PCR activities of the PolD constructs PolD-exo- and PolD-catalytic-core-exo-were compared on a 2.5 kb-long target DNA amplicon (FIG. 5). It was shown that the PolD-catalytic-core constructs, in particular the exo-version, are active in PCR at a wider range of polymerase concentrations than full-length PolD. While the PCR activity of full-length PolD is inhibited at higher concentrations of polymerase (40 μg/mL and above), the PolD-catalytic-core construct, in particular the exo-version, is still active at these higher concentrations of polymerase (from 40 μg/mL up to 1000 μg/mL) which are up to 50 times higher than the maximum effective concentration of PolD wild-type in the PCR conditions. These results suggest that deleting both the HTH and linker domains of DP1, as well the C-terminal domain of DP2, results in a substantially shorter enzyme, which remains active at a wider range of PCR conditions. The PolD-catalytic-core construct can therefore be used for a wider range of PCR applications. As the structure of DP1 and DP2 is highly conserved in archaea (FIGS. 1 and 2), similar results could be obtained using other PolD polymerases, in particular from hyperthermophilic Archaea of the order Thermococalles.

### Example 3: Reverse Transcriptase Activity

Previous studies reported the capacity of PolD to incorporate up to 4 NTPs in a DNA polymer using a DNA template (Zatopek et al., Nucleic acids Research, 2020, 48, 12204-12218) and to incorporate a dNTP when encountering a template that contains a single RNA base (Lemor et al., J. Mol. Biol., 2018, 430, 4908-4924).

The inventors have investigated the reverse transcriptase activity of the PolD constructs. To this end, fluorescent probes composed of a chimeric template strand and a fluorescence-labeled DNA primer were designed.

Primer: SEQ ID NO: 30:
5′-6FAM-GAGGTCTCGCTCCGACCGCTCCCG-3′;
Template DNA12: SEQ ID NO: 31:
5′-AGTGCCTAACGA-TG-CGGGAGCGGTCGGAGCGAGACCTC-3′;
Template RNA12: SEQ ID NO: 32:
5′-(AGUGCCUAACGA)-TG-CGGGAGCGGTCGGAGCGAGACCTC-3′
Template 2′-O-MeRNA12: SEQ ID NO: 33:
5′-[AGUGCCUAACGA]-TG-CGGGAGCGGTCGGAGCGAGACCTC-3′(;
Template DNA36: SEQ ID NO: 34:
5′-AGTGCCTAACCAAGTGCCTAACCAAGTGCCTAACGA-TG-
CGGGAGCGGTCGGAGCGAGACCTC-3′
Template RNA36: SEQ ID NO: 35:
5′-(AGUGCCUAACCAAGUGCCUAACCAAGUGCCUAACGA)-TG-
CGGGAGCGGTCGGAGCGAGACCTC-3′.

The template strand contains a 3′-primer-complementary-end made of DNA and a 5′-end presenting a various number of RNA or 2′-O-Methyl-RNA bases. If the tested polymerase presents a reverse transcription activity, it starts complementing the probes starting from the 3′-end of the primer and adds dNTPs corresponding to the RNA bases of the template strand. The presence of an enzymatic activity can be determined by visualization of the length of the primer on an acrylamide gel. The reaction mixture contains the PolD Reaction Buffer 1× (20 mM TrisHCl, pH9, 25 mM KCl, 10 mM (NH₄) 2SO₄, 2 mM MgCl₂, 1 mg/mL BSA, 0.1% v/v Tween20), 200 μM dNTPs, 100 nM fluorescent probe and the polymerase at 200 μg/mL. Mixes were incubated at 55° C. or 72° C. for 1 to 30 minutes and the reactions were stopped by adding 2 reaction volumes of loading buffer (10 mM EDTA, 1 mg/mL Bromophenol Blue, 90% deionized Formamide) on ice. Samples were Incubated subsequently at 95° C. for 5 minutes and loaded on an acrylamide gel.

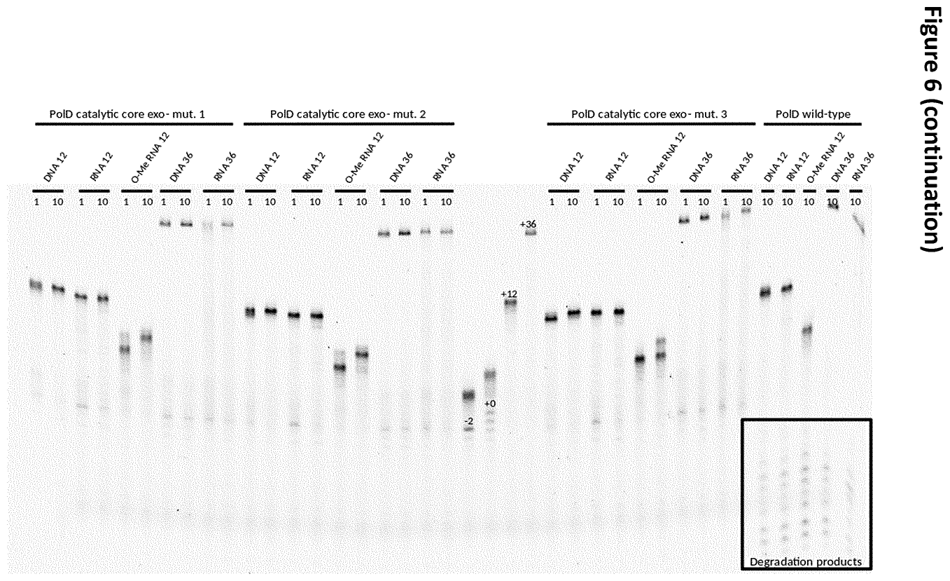

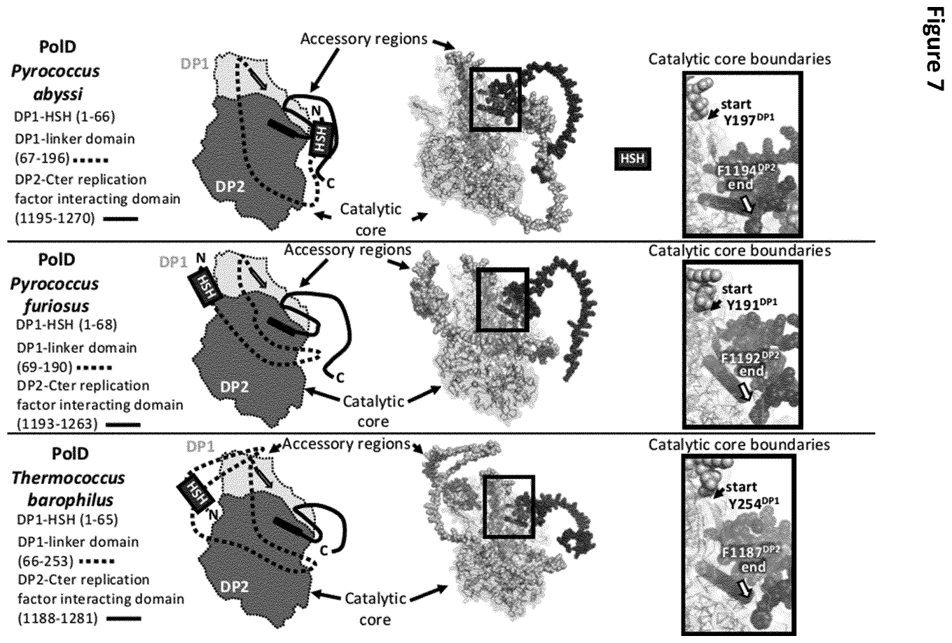

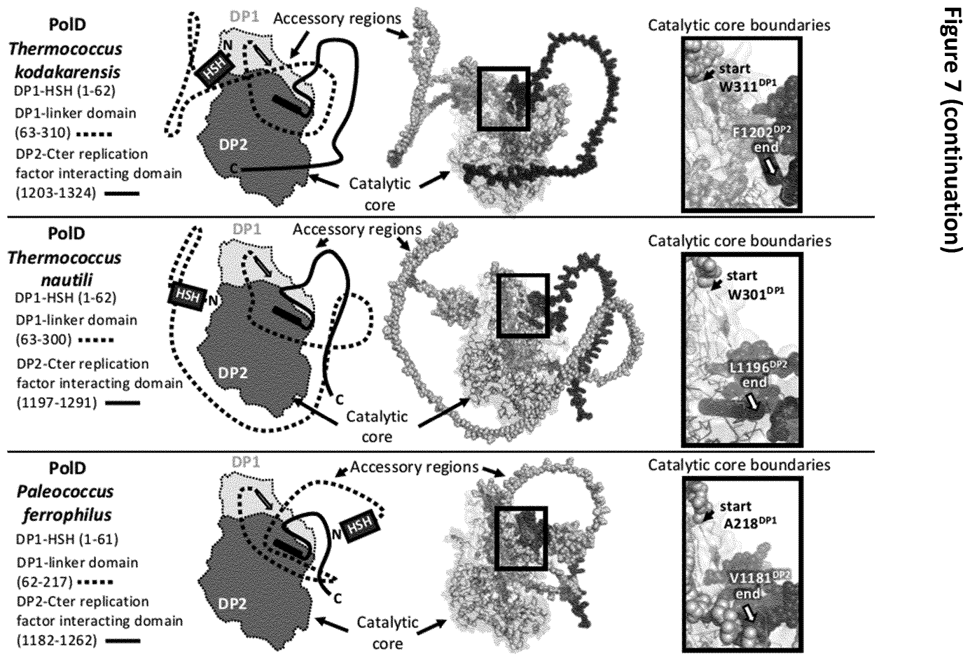

The reverse transcriptase activity assays show that PolD wild-type, PolD-exo-mut1, PolD-exo-mut2, PolD-exo-mut3 and the catalytic core PolD-catalytic-core-exo-mut1, PolD-catalytic-core-exo-mut2 and PolD-catalytic-core-exo-mut3 are able to fully reverse transcribe RNA 12-mers and 36-mers (FIG. 6). The three PolD constructs are also all able to incorporate up to 6 dNTPs for a 2′-O-Methyl-RNA template. On the other hand, a strong difference can be observed between the exo+ and exo− versions of the polymerases. The wild-type PolD degrades a lot the primer that it is supposed to elongate, the longer incubation the more, reducing the amount of fully elongated products compared with its three exo-versions. In conclusion, it was found that all PolD constructs have an unexpected reverse transcriptase activity that is more efficient in all three PolD exonuclease-deficient variants than in wild-type.

TABLE 1
Sequences disclosed in the present application

| SEQ | | | |
|---|---|---|---|
| ID | | | |
| NO: | Name | Type | Sequence |
| 1 | DP1 | PRT | MDELVKALERAGYLLTPSAYYLLVDHFKEGKFSLVELVKFAKSKGV |
| | Pyrococcus | | FIIDGDLAYEFLQFLGLGVPQEIKESYISTGEEAEKTVESQETRAS |
| | abyssi | | ELEEGGVSQVSSGELQELKEESPEISTTEEEIGGLELVQSSISTGS |
| | | | EVEYNNGENGESVVVLDKYGYPILYAPEEIGEEKEYSKYEDVVIEW |
| | | | NPSVTPVQIEKNYEVKFDVRQVKLRPPKVKNGSGKEGEIIVEAYAS |
| | | | LFKSRLSKLKRILRENPEISNVVDIGKLNYVSGDEEVTIIGLVNSK |
| | | | RETNRGLIFEVEDKTGIVKVFLPKDSEDYREAFKVLPDAVVAFKGF |
| | | | YSKKGIFFANKFYLPDVPLYRKQKPPLEEKVYAILISDIHVGSREF |
| | | | CEKAFLKFLEWLNGHVESKEEEEIVSRVKYLIIAGDVVDGIGIYPG |
| | | | QYSDLVIPDIFDQYEALANLLANVPEHITMFIGPGNHDAARPAIPQ |
| | | | PEFYKEYAKPIYKLKNAIIISNPAVIRLHGRDFLIAHGRGIEDVVS |
| | | | FVPGLTHHKPGLPMVELLKMRHLAPTFGGKVPIAPDPEDLLVIEEV |
| | | | PDLVQMGHVHVYDAVVYRGVQLVNSATWQAQTEFQKMVNIVPTPAK |
| | | | VPVVDVESARVVKVLDFSGWC |
| 2 | DP2 | PRT | MELPKEMEEYFEMLQREIDKAYEIAKKARAQGKDPSLDVEIPQATD |
| | Pyrococcus | | MAGRVESLVGPPGVAKRIRELVKEYGKEIAALKIVDEIIEGKFGDL |
| | abyssi | | GSREKYAEQAVRTALAILTEGIVSAPIEGIANVKIKRNTWADNSEY |
| | | | LALYYAGPIRSSGGTAQALSVLVGDYVRRKLGLDRFKPSEKHIERM |
| | | | VEEVDLYHRAVTRLQYHPSPEEVRLAMRNIPIEITGEATDDVEVSH |
| | | | RDVPGVETNQLRGGAILVLAEGVLQKAKKLVKYIDKMGIEGWEWLK |
| | | | EFVEAKEKGEPKEEGKEESLAESTLEETKVEVDMGFYYSLYQKFKE |
| | | | EIAPSDKYAKEVIGGRPLFSDPSKPGGFRLRYGRSRASGFATWGIN |
| | | | PATMILVDEFLAIGTQLKTERPGKGAVVTPVTTIEGPIVKLKDGSV |
| | | | LRVDDYNLALKVREDVEEILYLGDAVIAFGDFVENNQTLLPANYCE |
| | | | EWWILEFVKALKEIYEVHLEPFTENEEESIEEASDYLEIDPEF |
| | | | LKEMLRDPLRVKPPVELAIHFSEVLGIPLHPYYTLYWNSVEP |
| | | | KDVEKLWRLLKNYAEIEWSNFRGIKFAKKIVISQEKLGDSKR |
| | | | TLELLGLPHTVRDGNVIVDYPWAAALLTPLGNLNWEFMAKPL |
| | | | YATIDIINENNEIKLRDRGISWIGARMGRPEKAKERKMKPPV |
| | | | QVLFPIGLAGGSSRDIKKAAEEGKVAEVEIAFFKCPKCGHVG |
| | | | PEHLCPNCGTRKELLWVCPRCNAEYPESQAEGYNYTCPKCNVKLR |
| | | | PYAKRKIRPSELLNRAMENVKVYGVDKLKGVMGMTSGWKMPEPLEK |
| | | | GLLRAKNDVYVFKDGTIRFDATDAPITHFRPREIGVSVEKLRELGY |
| | | | THDFEGKPLVSEDQIVELKPQDIILSKEAGRYLLKVAKFVDDLLEK |
| | | | FYGLPRFYNAEKMEDLIGHLVIGLAPHTSAGIVGRIIGFVDALVGY |
| | | | AHPYFHAAKRRNCDGDEDAVMLLLDALLNFSRYYLPEKRGGKM |
| | | | DAPLVITTRLDPREVDSEVHNMDIVRYYPLEFYEATYELKSP |
| | | | KELVGVIERVEDRLGKPEMYYGLKFTHDTDDIALGPKMSLYK |
| | | | QLGDMEEKVRRQLEVAKRIRAVDEHGVAEKILNSHLIPDLRG |
| | | | NLRSFTRQEFRCVKCNTKFRRPPLNGKCPVCGGKIVLTVSKG |
| | | | AIEKYLGTAKMLVTEYNVKNYTRQRICLTERDIDSLFENVFP |
| | | | ETQLTLIVNPNDICQRLVMARTGEVNKSGLLENLSNGSKKTE |
| | | | KAEKAEKPRKKSDEKPKKKRVISLEEFFSRKSK |
| 3 | DP1 | PRT | MLIEDLIKNKYLITPSAYYLLEPHYKRDFTLAELIKFAKARGTFVI |
| | Thermococcus | | DSSIAEAFLAEKGLFSTGELTEQTPLEVSEEEPLEASPEEIHGDFA |
| | nautili | | ESAEIPVSETVTASRPEEIPSSEDFESAEASGSISTGDVVESEVAT |
| | | | ASSGEESVEPAEIVGETSISTGTTLELEQSPASIPVEEPISSEISE |
| | | | GESFVSTGTPEHEELVDEAAGSLDSSLTEVVEDSLPVEAEPLNGNG |
| | | | YGNYADSEEYYENGENGNGVKPKIVYGDYGIPIAYVGEDVEGEKSY |
| | | | STYEDVVIKPKEGFHYRAKEIPDEWELTFDVKNVKFEAPKVKNAAS |
| | | | KEGEIIIQAYSSYFKSRLRKMRRIFRENPEIGTVIDIAKLSYVRDD |
| | | | EVTIIGLVNDKRETAKGYMFEVEDATGRVKVFLGRDKENASQAYNA |
| | | | IMPDSVVAFRGQPGRGIFFANRVFLPDVPKFRREKPPLEEKVYAIL |
| | | | LSDIHVGSNKFCEKAFEKFLEWLNGEVNSRAEEELVSRIKYLIIGG |
| | | | DVVDGVGIYPGQYNELAIPDIFDQYEALANLLRNVPEHITMFIGPG |
| | | | NHDAARTALPQPGFYEEYAKPIYKLKNAVILSNPAVINLHGREFLI |
| | | | AHGRGIEDVVDFIPGRTHHKPAEAMVDLLKLRHLAPTFGNKVPIAP |
| | | | DPEDTLVIESVPDLFQAGHVHVMEYRIYNGVFVINSGTWQAQTEFQ |
| | | | KMVNIIPTPARVPIIDVETARLRAVVSFDQFCEGV |
| 4 | DP1 | PRT | MLVEDLLKNNYLITPSAYYLLSDHYKKAFTLAELIKFAKNRGTFVV |
| | Thermococcus | | DSNLAREFLAEKGIISSGNGALDGSVLSGISVSESPVKPNGEAVLE |
| | kodakarensis | | SESDESGAIESSLQEGVEDGVESIAEPSAASVEDVVVAGESSLASE |
| | | | GSSLEDEINTEPELSSSMDDGAEVVQSSEGDETSISTGNALGSVDY |
| | | | GGSDSESEALEEASSGGESFISTGTPEDEALLEESPEGEFPDENGF |
| | | | SDESLPVENGSENGYGDSEEYYENGDNGVKPKIVYGDYGVPIAYVA |
| | | | DETPEEEKAYSTYSDLVIAPKEGFHYRAKEIPDEWELAFDVKNVKF |
| | | | EVPKVKNAQSKEGEVIIQAYSSYFKSRLKKMRRIFRENPEIGTIVD |
| | | | IAKLSYVREDDVTIIGLVNEKRETRKGYLFEIEDATGRIKVFIGSD |
| | | | KEGANEAYSTIMPDSVVAFRGTPGKGIFFANRVFLPDVPKFKRSKP |
| | | | PLEEKVYAILLSDIHVGSNKFCEEAFIKFLEWLNGEVNSRTEEE |
| | | | LVSRIKYIIIGGDVVDGVGIYPGQYNELAIPDIFDQYEALAN |
| | | | LLKQVPDHITMFIGPGNHDAARTALPQPGFYEEYAKPLYKLK |
| | | | NAVIISNPAVIRLHGRDFLVAHGRGIEDVVDFVPNRSHHRPA |
| | | | EAMVELLKLRHIAPTFGNKVPIAPDPEDTLVIESVPDLFQAG |
| | | | HVHVMQYKTYNGVFVINTGTWQAQTEFQKMVNIIPTPARVPI |
| | | | IDVETA |
| | | | RLRAVVREDQFCEGV |
| 5 | DP1 | PRT | MKLVEDLIKNNYLITPPAYYTLAHHYENGSFTLAELVKFAKSRGTF |
| | Palaeococcus | | VIDEGTAEEFIKWKGLISSGANGEHESVEEPSVEGMESGTEGAVPQ |
| | ferrophilus | | EKELLMGESAGVENSSIEVTAVEESPAVESAPESPEFSNEDVQMEE |
| | | | PLTNGDETLVSNGTWAGSNGDYNGDEGAVKAVYSDYGVLVPLEEEP |
| | | | PEEIGAKSYSVYADFKVTPREGFRPRAAEMPSEARVEFDVRNVKLT |
| | | | PPKVKNAASKEGEIIIRSYELHFRSRLRKLRRILRDTNELTGVVDI |
| | | | GRLSYTAGYEDPVTIIGLVNSKRETRKGFMFELEDATGTVKVFIKR |
| | | | DNPDYSKVWEVLLDSVVAFSGYYSKRGLFFAEHIYLPDVPLYRREK |
| | | | PPLEEPVYAALISDIHVGSDTFCEKAFMKFMKWLNGYTASREEEEI |
| | | | LDRLKYLIIAGDVVDGVGIYPGQYNELLIPDIFDQYEALANLLSNV |
| | | | PEHIQVFIGPGNHDAARTALPQPGFYEEYAYPLLKLKNTTIIS |
| | | | NPAVLDLHGRKFLIAHGRGIEDIVGNVPNTSHHEPARPMVEL |
| | | | LKLRHLAPTFGGKVPVAPDPEDLLVIEEVPDFVQMGHVHVLD |
| | | | YQVYRGVFLLNSATWQAQTEFQKMVNIVPTPAKVPIVDVGTA |
| | | | RLKTVVDFSKWCEGM |
| 6 | DP1 | PRT | MNELVKDLLSNRYLITPPAYFILSEYYKKSFTLAELIKFARTKNTF |
| | Thermococcus | | VIDERLAKEFLESKGLLSPLESFTKYTQKVPVETKAETPPSIAEVP |
| | barophilus | | MEISISGGSKEISIQSAVSGEKTAKLGKMVEIPPQASDKTEIAHDI |
| | | | QELPSEKGTSISTGEPMENMVSETISTEIVEEEPTIEGGESFISTG |
| | | | APSPEEYTGAGDEENGVKMKAVYGDYGVLVPLEEEEIPAEERRAYS |
| | | | IYEDFKVELNESFEFVAKRIKVEYEIKFDVRNVKLKPPKAKNANGK |
| | | | EGEIVVKVYENYFKSRLKKLRRILRENPEVSGVIDIGKLSYINPDG |
| | | | EVTIIGLVNSKRETKKGFIFEVEDMTGIVKVFLGRDNEDYKKAFEV |
| | | | LPDSVVAFRGYYSKRGILFASKLYLPDVPIYKREKPPLEEKVYAVL |
| | | | ISDIHVGSTKFCEKAFMKFLEWLNGNVESREEEEIVSRIKYMIIAG |
| | | | DVVDGIGIYPGQYNELAIPDIFDQYEALANLLANVPDHITMFI |
| | | | GPGNHDAARTALPQPEFYREYAKPLYELKNAVIISNPAVINL |
| | | | HGRDFLIVHGRGIEDVVSYIPGKSHLHPVEPMIELLKLRHVA |
| | | | PTFGGKVPIAPDPEDLLVIEEVPDLVQMGHVHVLDYQIYRGV |
| | | | FLINSATWQAQTEFQKMVNIVPTPARVPIIDIETARLKTVVD |
| | | | FSRWCE |
| 7 | DP1 | PRT | MDEFVKSLLKANYLITPSAYYLLREYYEKGEFSIVELVKFARSRES |
| | Pyrococcus | | YIITDALATEFLKVKGLEPILPVETKGGFVSTGESQKEQSYEESFG |
| | furiosus | | TKEEISQEIKEGESFISTGSEPLEEELNSIGIEEIGANEELVSNGN |
| | | | DNGGEAIVFDKYGYPMVYAPEEIEVEEKEYSKYEDLTIPMNPDFNY |
| | | | VEIKEDYDVVFDVRNVKLKPPKVKNGNGKEGEIIVEAYASLFRSRL |
| | | | KKLRKILRENPELDNVVDIGKLKYVKEDETVTIIGLVNSKREVNKG |
| | | | LIFEIEDLTGKVKVFLPKDSEDYREAFKVLPDAVVAFKGVYSKRGI |
| | | | LYANKFYLPDVPLYRRQKPPLEEKVYAILISDIHVGSKEFCENAFI |
| | | | KFLEWLNGNVETKEEEEIVSRVKYLIIAGDVVDGVGVYPGQYADLT |
| | | | IPDIFDQYEALANLLSHVPKHITMFIAPGNHDAARQAIPQPEFYKE |
| | | | YAKPIYKLKNAVIISNPAVIRLHGRDFLIAHGRGIEDVVGSVP |
| | | | GLTHHKPGLPMVELLKMRHVAPMFGGKVPIAPDPEDLLVIEE |
| | | | VPDVVHMGHVHVYDAVVYRGVQLVNSATWQAQTEFQKMVNIV |
| | | | PTPAKVPVVDIDTAKVVKVLDFSGWC |
| 8 | DP2 | PRT | MGEELYSPEMKAYFESLQREIDRAYEIARKARSQGKDPSLDVEVPQ |
| | Thermococcus | | ATDMAGRVESLVGPPGVAERIRELVKEYGKELAALKVVDEIIEGKF |
| | nautili | | GDLGSKEKYAEQAVRTALAILTEGIVSAPLEGIADVKIKRNTWADN |
| | | | SEYLALYYAGPIRSSGGTAQALSVLVGDYVRKKLGLDRFKPSEEHI |
| | | | ERMVEEIDLYHRAVTRLQYHPEADEVRLAMRNIPIEITGEETDKVE |
| | | | VSHRNVPGVETNHLRGGAILVLAEGVLQKAKKLVKYIDKMGVEGWD |
| | | | WIKEFVEAKEKGKSSEEPKVDESKAEDSGAREEVAEKVEKGFYYEL |
| | | | YERFRANIAPNKKYTKEIIGGRPLFAEPSENGGFRLRYGRSRVSGF |
| | | | ATWSVNPATMLILDEFIAIGTQMKTERPGKGCIVTPATTVEGPIVK |
| | | | LKDGSVVRVDDYETALRVKNEIEEILYVGDALVNFGDFVENNQTLL |
| | | | PANYVEEWWVQELVKAIEELYEVELKPFSDNPREAVEEAAEYLEVD |
| | | | PDFLESLLRDPLRVKPSVELAIHLSKVLDIPFHPYYTLYWNTLKPE |
| | | | EVEELQKALLNAQIEWDEFRKLKFAKRVILENDPQIKRYLELLGLP |
| | | | HRLERTEDRRKVIVIDYPWSSALLTPLGDLEWEFKAKPFFTVIDII |
| | | | NESNPIKLRDRGISWIGARMGRPEKAKERKMKPPVQVLFPIGLAGG |
| | | | QSRDIKKAAEEGKTAKVEIAFFKCPKCGHTGPEHLCPVCGTRKELL |
| | | | WHCPKCNVDYPESEAKEFGFRCPRCDVELKPYAEREIKPSELLRRA |
| | | | MDNVKVYGIDRLKGVKGMTSGYKMAEPLEKGLLRVKNDVYVFKDGT |
| | | | IRFDATDAPITHFKPKEIGVSVEKLRELGYTHDFEGKPLERDDQIV |
| | | | ELKVQDIILSYEAGKYLLKVARFVDDLLEKFYGLPRFYNAEKMEDL |
| | | | IGHLVIGLAPHTSAGIIGRIIGFSDVLVGYAHPYYHAAKRRNCDGD |
| | | | EDAVMLLLDALLNFSKYYLPEKRGGKMDAPLVVTTRLDPREVDSEV |
| | | | HNMDVVRYYPLEFYEVTYDMKSPKEIKFIERVEDRLGKPEMYEGIK |
| | | | FTHDTDDIGLGPKMSLYKQLGDMVEKVERQLALAERIRAVDEHHVA |
| | | | ETILNSHLIPDLRGNLRSFTRQEFRCVKCNTKYRRPPLIGRCPKCG |
| | | | GKIVLTVSKGAVEKYLPTAKMLVTYKVKDYTRQRICLTEKDIKTLF |
| | | | ETVLPEKQRTLLGFSADVCEKMIKERTGSSNGKNGYLDGFNGKNGK |
| | | | AKKAKVGSEAKKEKKPKTEKSKDLKKEVKKEKAKARRKRISLDEFF |
| | | | GG |
| 9 | DP2 | PRT | MSEEIYSPEMKAYFESLQREIDRAYAIARKARAQGKDPSFDVEVPQ |
| | Thermococcus | | ATDMAGRVESLVGPPGVAERIRELVKEYGKEIAALKVVDEIIEGKF |
| | kodakarensis | | GDLGSKEKYAEQAVRTALAILTEGIVSAPLEGIADVKIKRNEWADG |
| | | | SEYLALYYAGPIRSSGGTAQALSVLVGDYVRRKLGLDRFKPSDEHI |
| | | | ERMVEEVDLYHRAVTRLQYHPEADEVRLAMRNIPIEITGEETDKVE |
| | | | VSHRNVPGVETNHLRGGAILVLAEGVLQKAKKLVKYIDKMGIEGWD |
| | | | WIKEFVEAKEKGKSSEENKDESKAEDTGTESVAEKKENVEKGFYYE |
| | | | LYEKFRANIAPNKKYTKEIIGGRPLFAEPSTNGGFRLRYGRSRVSG |
| | | | FATWSVNPATMLILDEFIAIGTQMKTERPGKGCIVTPATTVEGPIV |
| | | | RLKNGSVVRVDDYETALKVRNEVDEILYVGDALVNFGDFVENNQTL |
| | | | LPANYVEEWWVQELVQAIKDLYEVELQPFAENDREAVEEAAEYLEV |
| | | | DPDFLWNLLKDPLRVKPDVETAIHLSTVLDIPFHPYYTLYWNTLQP |
| | | | EEVEELQKALLGAQIEWAEFRKNRFAKKVVLENDKNIKRYLELLGL |
| | | | PHRLERVEKKRKVIVVEYPWSAALLTPLGNLEWEFKAKPFYTVIDI |
| | | | INENNRIKLRDRGISWIGARMGRPEKAKERKMKPPVQVLFPIGLAG |
| | | | GQSRDIKKAAEEGKTARVEIAFFKCPKCGHVGPEHLCPVCGTRKEL |
| | | | LWHCPKCGADYPESDAKDFNYRCPKCDVELKPYAEREIKPADLLRQ |
| | | | AMDNVKVYGIDRLKGVKGMTSGYKMAEPLEKGLLRVKNDVYVFKDG |
| | | | TIRFDATDAPITHFKPKEIGTSVEKLRELGYTHDFEGKPLERDDQI |
| | | | LELKVQDVILPYEAGRYLLKVARFIDDLLEKFYGLPRFYNAEKMED |
| | | | LVGHLVIGLAPHTSAGIIGRIIGFSDVLVGYAHPYYHAAKRRNCDG |
| | | | DEDAVMLLLDALLNFSKYYLPEKRGGKMDAPLVVTTRLDPREVDSE |
| | | | VHNMDVVRYYPLEFYKATYELKSPKEVKVIERVEDRLGKPEMYEGI |
| | | | KFTHDTDDIGLGPKMSLYKQLGDMEEKVARQLALAERIRAVDEHHV |
| | | | AETIINSHLVPDLRGNLRSFTRQEFRCVKCNTKYRRPPLTGKCPKC |
| | | | GGKIVLTVSKGAIEKYLPTAKMLVTKYRVKDYTRQRICITEKDIKT |
| | | | LFENVFPEKQRTLMGFSADICEKMVKERTGHSNGKNGYLDEFNGKN |
| | | | GKASKKSGSLASKLSGKGKEPSKKKESAKPKRSEKVKNLTSFEAAA |
| | | | KNEQARGTAGNAKKAESEKPKRKKRKGISLDEFFGS |
| 10 | DP2 | PRT | MTELYSDEMKAYFESLQREIDRAYEIAEKARAQGKDPVPEIEVPQA |
| | Palaeococcus | | TDMAGRVESLVGPKGVAERIRALVKEHGKELAALKIVDEIIEGRFG |
| | ferrophilus | | ELGDEETLAEQAVRTSLAVLTEGIVSAPLEGIAFVKVRQNFDGSRY |
| | | | LALYYAGPIRSSGGTAQALSVLVGDYVRKKLGLDRYKPTPEEIERY |
| | | | VEEVDLYHRAVSRLQYHPSADEVRLAIKSIPVEITGEATDQVEVSG |
| | | | YRNLPRVETNQLRGGAILVLAEGVLQKAKKLLKYIDKLEVEGWEWL |
| | | | REFVESKESGKGKEKQEVVEESAEEETTAEVEMGFYYRLYEEFRKK |
| | | | IAPNKKYTKEIIGGRPLFAEPSTNGGFRLRYGRSRTSGFATWSVHP |
| | | | ATMLLLDEFIAVGTQMKTERPGKGCITTPATTIEGPIVKLRDGSVV |
| | | | RVEDYETALRVRDSIEEILYVGDALVNFGDFVENNQTLLPANYAEE |
| | | | WWIQEFVKAVKDVYEVELKPFEENDGEDVEEAAEYLDVEPDFLGRL |
| | | | LYDPLRVRPRVEEAIHFSRVLRIPLHPYYTLYWNTLGVEELLALQE |
| | | | GLVDASIEWDSFRGVKFAKRLTTRNFSIKRSLELLGLPHRVENGEI |
| | | | VVEYPWSAALLTPLLNLERRLEPKPFHTPIDIINAVSDVKLRDRGM |
| | | | SWIGARMGRPEKAKERKMKPPVHVLFPIGLAGGSSRDIYKAFQEGK |
| | | | STTVEIARFKCPSCGHVDIFPRCPKCGSRTHLYTCPSCGHTSTEER |
| | | | VCPKCKVEMKPYAKTTFKIGDYLRPAMDNVKVYTLDRLKGVLGMSS |
| | | | AFKLAEPLEKGLLRAKNDVYVFKDGTIRFDATDAPVTHFRPREIGT |
| | | | SVEKLRELGYTHDFEGKPLESEDQILELKVQDVILPKTAGEYLLKV |
| | | | ANFIDDLLERFYGLPRFYNAEKMEDLVGHLVIGLAPHTSAGIMGRI |
| | | | IGFVDALVGYAHPYYHAAKRRNCDGDEDAVMLLLDALLNFSKYYLP |
| | | | EKRGGKMDAPLVVTTRLDPREVDSEVHNMDVVRYYPLEFYRATYEL |
| | | | KSPKEVKVIERVEDRLGKPEMYEGIKFTHDSDDIALGPKMSLYKQL |
| | | | GDMEDKVRRQLDLAKRIRAVDEHHVAETIINSHLIPDLRGNLRSFT |
| | | | RQEFRCVKCNTKYRRPPMSGKCPKCGGKIVLTVSKGAIEKYLSTAK |
| | | | MLVTEFDVLDYTRQRICITEKDIDTLFTNVFPRVQTTLMVATPKDV |
| | | | CSTMIARRRGTLKEKPKKSNGKSWDPSKDFKKPNGEAPKNGESKPA |
| | | | KAPKKKKVIGLDEFFGKKS |
| 11 | DP2 | PRT | MSELYSEEMKAYFEMLQKEIDKAYEIAKKARAQGKDPVRDVEIPQA |
| | Thermococcus | | TDMAGRVESLVGPKGVAERIRELAREYGKELAALKIVDEIIEGKFG |
| | barophilus | | KFDSKEKLAEQAVRTALAILTEGIVSAPIEGIAQVKIKKNPDGTEY |
| | | | LALYYAGPIRSSGGTAQALSVLVGDYVRKKLGLDKFKPTEKHIERM |
| | | | VEEVDLYHRAVTRLQYHPSADEVRLAMQNIPVEITGEATDDVEVSH |
| | | | RDVPGVETNQLRGGAILVLAEGVLQKAKKLVKYIDKMGIEGWGWLK |
| | | | EFVEAKEKGKTEEGTEEKAEKSKGEEAEIQTSVKVKKGFYYELYEK |
| | | | FRANIAPNNKYTKEIIGGRPLFAEPSENGGFRLRYGRSRVSGFATW |
| | | | SINPAVMILVDEFLAVGTQMKTERPGKGCIVTPATTAEGPIVKLKD |
| | | | GSVIRVDDYYLALKIRDQVEEILYLGDAIIAFGDFVENNQTLLPAN |
| | | | YVEEWWIQEFVKAVEDIYEVPLKPFAENDEETVEEAADYLDLKPGF |
| | | | LAELLKDPLKIRPKVEEAIHLSKVLDIPLHPYYTLYWNTLKPEEVA |
| | | | ELQSLLVNAEIEWDKYLGTKYAKKIVINLEVLGREGKRYLELLGLP |
| | | | HRVEDKSVIIEYPWSAALLTPLANLEKKFEPKEFHTTIDIVNEVSE |
| | | | VKLRDRGISWIGARMGRPEKAKERKMKPPVHVLFPIGLAGGSSRDI |
| | | | YKAAQEGKLAEVEIAQFKCTNCGHIDIFPTCPVCGAEAKLLYRCPK |
| | | | CNYSSTEETICPKCGIEMRTYVKKTINPSHLVKQAMQNVRVNTLDK |
| | | | LKGVMGMTSAHKVPEPLEKGILRAKNDVYVFKDGTIRFDATDAPIT |
| | | | HFKPREIGVSVEKLRELGYTHDFEGKPLVSEDQIVELKVQDVILSK |
| | | | EAGKYLVRVAKFIDDLLERFYGLPRFYNVEKMEDLIGHLVIGLAPH |
| | | | TSAGIIGRIIGFVDALVGYAHPYYHAAKRRNCDGDEDSVMLLMDAL |
| | | | LNFSRYYLPEKRGGKMDAPLVVTTRLDPREVDSEVHNMDIARYYPL |
| | | | EFYEATYELKSPKELVGVVERVEDRLGKPEMYEGLKFTHDTDDIAL |
| | | | GPKLSKYKQLGDMEEKVRQQLALAEKIRAVNEHHVAETIINSHLIP |
| | | | DLRGNLRSFTRQEFRCVKCNTKYRRPPLSGKCPKCGGKIVLTVSKG |
| | | | AIEKYLPTAKLLVTNYDVLTYTRQRICLTEKDIKSLFANVFPETQR |
| | | | TLLALNTNDICDRMIAERTGKIVTKNGYLDEFHGNGRKVQKKVEAP |
| | | | KKVEKRELTKMQAHKSEKPGKHKPKKKKKVVSLDEFFGA |
| 12 | DP2 | PRT | MELPKEIEEYFEMLQREIDKAYEIAKKARSQGKDPSTDVEIPQATD |
| | Pyrococcus | | MAGRVESLVGPPGVAQRIRELLKEYDKEIVALKIVDEIIEGKFGDF |
| | furiosus | | GSKEKYAEQAVRTALAILTEGIVSAPLEGIADVKIKRNTWADNSEY |
| | | | LALYYAGPIRSSGGTAQALSVLVGDYVRRKLGLDRFKPSGKHIERM |
| | | | VEEVDLYHRAVSRLQYHPSPDEVRLAMRNIPIEITGEATDDVEVSH |
| | | | RDVEGVETNQLRGGAILVLAEGVLQKAKKLVKYIDKMGIDGWEWLK |
| | | | EFVEAKEKGEEIEESESKAEESKVETRVEVEKGFYYKLYEKFRAEI |
| | | | APSEKYAKEIIGGRPLFAGPSENGGFRLRYGRSRVSGFATWSINPA |
| | | | TMVLVDEFLAIGTQMKTERPGKGAVVTPATTAEGPIVKLKDGSVVR |
| | | | VDDYNLALKIRDEVEEILYLGDAIIAFGDFVENNQTLLPANYVEEW |
| | | | WIQEFVKAVNEAYEVELRPFEENPRESVEEAAEYLEVDPEFLAKML |
| | | | YDPLRVKPPVELAIHFSEILEIPLHPYYTLYWNTVNPKDVERLWGV |
| | | | LKDKATIEWGTFRGIKFAKKIEISLDDLGSLKRTLELLGLPHTVRE |
| | | | GIVVVDYPWSAALLTPLGNLEWEFKAKPFYTVIDIINENNQIKLRD |
| | | | RGISWIGARMGRPEKAKERKMKPPVQVLFPIGLAGGSSRDIKKAAE |
| | | | EGKIAEVEIAFFKCPKCGHVGPETLCPECGIRKELIWTCPKCGAEY |
| | | | TNSQAEGYSYSCPKCNVKLKPFTKRKIKPSELLNRAMENVKVYGVD |
| | | | KLKGVMGMTSGWKIAEPLEKGLLRAKNEVYVFKDGTIRFDATDAPI |
| | | | THFRPREIGVSVEKLRELGYTHDFEGKPLVSEDQIVELKPQDVILS |
| | | | KEAGKYLLRVARFVDDLLEKFYGLPRFYNAEKMEDLIGHLVIGLAP |
| | | | HTSAGIVGRIIGFVDALVGYAHPYFHAAKRRNCDGDEDSVMLLLDA |
| | | | LLNFSRYYLPEKRGGKMDAPLVITTRLDPREVDSEVHNMDVVRYYP |
| | | | LEFYEATYELKSPKELVGVIERVEDRLGKPEMYYGIKFTHDTDDIA |
| | | | LGPKMSLYKQLGDMEEKVKRQLTLAERIRAVDQHYVAETILNSHLI |
| | | | PDLRGNLRSFTRQEFRCVKCNTKYRRPPLDGKCPVCGGKIVLTVSK |
| | | | GAIEKYLGTAKMLVANYNVKPYTRQRICLTEKDIDSLFEYLFPEAQ |
| | | | LTLIVDPNDICMKMIKERTGETVQGGLLENFNSSGNNGKKIEKKEK |
| | | | KAKEKPKKKKVISLDDFFSKR |
| 13 | DP1-ΔN (1- | PRT | MGENGESVVVLDKYGYPILYAPEEIGEEKEYSKYEDVVIEWNPSVT |
| | 144) | | PVQIEKNYEVKFDVRQVKLRPPKVKNGSGKEGEIIVEAYASLFKSR |
| | | | LSKLKRILRENPEISNVVDIGKLNYVSGDEEVTIIGLVNSKRETNR |
| | | | GLIFEVEDKTGIVKVFLPKDSEDYREAFKVLPDAVVAFKGFYSKKG |
| | | | IFFANKFYLPDVPLYRKQKPPLEEKVYAILISDIHVGSREFCEKAF |
| | | | LKFLEWLNGHVESKEEEEIVSRVKYLIIAGDVVDGIGIYPGQYSDL |
| | | | VIPDIFDQYEALANLLANVPEHITMFIGPGNHDAARPAIPQPEFYK |
| | | | EYAKPIYKLKNAIIISNPAVIRLHGRDFLIAHGRGIEDVVSFVPGL |
| | | | THHKPGLPMVELLKMRHLAPTFGGKVPIAPDPEDLLVIEEVPDLVQ |
| | | | MGHVHVYDAVVYRGVQLVNSATWQAQTEFQKMVNIVPTPAKVPVVD |
| | | | VESARVVKVLDFSGWC |
| 14 | DP1-ΔN (1- | PRT | MGENGESVVVLDKYGYPILYAPEEIGEEKEYSKYEDVVIEWNPSVT |
| | 144) -H451A | | PVQIEKNYEVKFDVRQVKLRPPKVKNGSGKEGEIIVEAYASLFKSR |
| | | | LSKLKRILRENPEISNVVDIGKLNYVSGDEEVTIIGLVNSKRETNR |
| | | | GLIFEVEDKTGIVKVFLPKDSEDYREAFKVLPDAVVAFKGFYSKKG |
| | | | IFFANKFYLPDVPLYRKQKPPLEEKVYAILISDIHVGSREFCEKAF |
| | | | LKFLEWLNGHVESKEEEEIVSRVKYLIIAGDVVDGIGIYPGQYSDL |
| | | | VIPDIFDQYEALANLLANVPEHITMFIGPGNADAARPAIPQPEFYK |
| | | | EYAKPIYKLKNAIIISNPAVIRLHGRDFLIAHGRGIEDVVSFVPGL |
| | | | THHKPGLPMVELLKMRHLAPTFGGKVPIAPDPEDLLVIEEVPDLVQ |
| | | | MGHVHVYDAVVYRGVQLVNSATWQAQTEFQKMVNIVPTPAKVPVVD |
| | | | VESARVVKVLDFSGWC |
| 15 | DP2-ΔC (1217- | PRT | MELPKEMEEYFEMLQREIDKAYEIAKKARAQGKDPSLDVEIPQATD |
| | 1270) | | MAGRVESLVGPPGVAKRIRELVKEYGKEIAALKIVDEIIEGKFGDL |
| | construct | | GSREKYAEQAVRTALAILTEGIVSAPIEGIANVKIKRNTWADNSEY |
| | | | LALYYAGPIRSSGGTAQALSVLVGDYVRRKLGLDRFKPSEKHIERM |
| | | | VEEVDLYHRAVTRLQYHPSPEEVRLAMRNIPIEITGEATDDVEVSH |
| | | | RDVPGVETNQLRGGAILVLAEGVLQKAKKLVKYIDKMGIEGWEWLK |
| | | | EFVEAKEKGEPKEEGKEESLAESTLEETKVEVDMGFYYSLYQKFKE |
| | | | EIAPSDKYAKEVIGGRPLFSDPSKPGGFRLRYGRSRASGFATWGIN |
| | | | PATMILVDEFLAIGTQLKTERPGKGAVVTPVTTIEGPIVKLKDGSV |
| | | | LRVDDYNLALKVREDVEEILYLGDAVIAFGDFVENNQTLLPANYCE |
| | | | EWWILEFVKALKEIYEVHLEPFTENEEESIEEASDYLEIDPEFLKE |
| | | | MLRDPLRVKPPVELAIHFSEVLGIPLHPYYTLYWNSVEPKDVEKLW |
| | | | RLLKNYAEIEWSNFRGIKFAKKIVISQEKLGDSKRTLELLGLPHTV |
| | | | RDGNVIVDYPWAAALLTPLGNLNWEFMAKPLYATIDIINENNEIKL |
| | | | RDRGISWIGARMGRPEKAKERKMKPPVQVLFPIGLAGGSSRDIKKA |
| | | | AEEGKVAEVEIAFFKCPKCGHVGPEHLCPNCGTRKELLWVCPRCNA |
| | | | EYPESQAEGYNYTCPKCNVKLRPYAKRKIRPSELLNRAMENVKVYG |
| | | | VDKLKGVMGMTSGWKMPEPLEKGLLRAKNDVYVFKDGTIRFDATDA |
| | | | PITHFRPREIGVSVEKLRELGYTHDFEGKPLVSEDQIVELKPQDII |
| | | | LSKEAGRYLLKVAKFVDDLLEKFYGLPRFYNAEKMEDLIGHLVIGL |
| | | | APHTSAGIVGRIIGFVDALVGYAHPYFHAAKRRNCDGDEDAVMLLL |
| | | | DALLNFSRYYLPEKRGGKMDAPLVITTRLDPREVDSEVHNMDIVRY |
| | | | YPLEFYEATYELKSPKELVGVIERVEDRLGKPEMYYGLKFTHDTDD |
| | | | IALGPKMSLYKQLGDMEEKVRRQLEVAKRIRAVDEHGVAEKILNSH |
| | | | LIPDLRGNLRSFTRQEFRCVKCNTKFRRPPLNGKCPVCGGKIVLTV |
| | | | SKGAIEKYLGTAKMLVTEYNVKNYTRQRICLTERDIDSLFENVFPE |
| | | | TQLTLIVNPNDICQRLVMAR |
| 16 | DP1 wt | PRT | MGKHHHHSGHHHTGHHHHSGSHHHTSSSASTGENLYFQGTGDGSDE |
| | construct | | LVKALERAGYLLTPSAYYLLVDHFKEGKFSLVELVKFAKSKGVFII |
| | | | DGDLAYEFLQFLGLGVPQEIKESYISTGEEAEKTVESQETRASELE |
| | | | EGGVSQVSSGELQELKEESPEISTTEEEIGGLELVQSSISTGSEVE |
| | | | YNNGENGESVVVLDKYGYPILYAPEEIGEEKEYSKYEDVVIEWNPS |
| | | | VTPVQIEKNYEVKFDVRQVKLRPPKVKNGSGKEGEIIVEAYASLFK |
| | | | SRLSKLKRILRENPEISNVVDIGKLNYVSGDEEVTIIGLVNSKRET |
| | | | NRGLIFEVEDKTGIVKVFLPKDSEDYREAFKVLPDAVVAFKGFYSK |
| | | | KGIFFANKFYLPDVPLYRKQKPPLEEKVYAILISDIHVGSREFCEK |
| | | | AFLKFLEWLNGHVESKEEEEIVSRVKYLIIAGDVVDGIGIYPGQYS |
| | | | DLVIPDIFDQYEALANLLANVPEHITMFIGPGNHDAARPAIPQPEF |
| | | | YKEYAKPIYKLKNAIIISNPAVIRLHGRDFLIAHGRGIEDVVSFVP |
| | | | GLTHHKPGLPMVELLKMRHLAPTFGGKVPIAPDPEDLLVIEEVPDL |
| | | | VQMGHVHVYDAVVYRGVQLVNSATWQAQTEFQKMVNIVPTPAKVPV |
| | | | VDVESARVVKVLDFSGWC |
| 17 | DP1-H451A | PRT | MGKHHHHSGHHHTGHHHHSGSHHHTSSSASTGENLYFQGTGDGSDE |
| | construct | | LVKALERAGYLLTPSAYYLLVDHFKEGKFSLVELVKFAKSKGVFII |
| | | | DGDLAYEFLQFLGLGVPQEIKESYISTGEEAEKTVESQETRASELE |
| | | | EGGVSQVSSGELQELKEESPEISTTEEEIGGLELVQSSISTGSEVE |
| | | | YNNGENGESVVVLDKYGYPILYAPEEIGEEKEYSKYEDVVIEWNPS |
| | | | VTPVQIEKNYEVKFDVRQVKLRPPKVKNGSGKEGEIIVEAYASLFK |
| | | | SRLSKLKRILRENPEISNVVDIGKLNYVSGDEEVTIIGLVNSKRET |
| | | | NRGLIFEVEDKTGIVKVFLPKDSEDYREAFKVLPDAVVAFKGFYSK |
| | | | KGIFFANKFYLPDVPLYRKQKPPLEEKVYAILISDIHVGSREFCEK |
| | | | AFLKFLEWLNGHVESKEEEEIVSRVKYLIIAGDVVDGIGIYPGQYS |
| | | | DLVIPDIFDQYEALANLLANVPEHITMFIGPGNADAARPAIPQPEF |
| | | | YKEYAKPIYKLKNAIIISNPAVIRLHGRDFLIAHGRGIEDVVSFVP |
| | | | GLTHHKPGLPMVELLKMRHLAPTFGGKVPIAPDPEDLLVIEEVPDL |
| | | | VQMGHVHVYDAVVYRGVQLVNSATWQAQTEFQKMVNIVPTPAKVPV |
| | | | VDVESARVVKVLDFSGWC |
| 18 | DP1-ΔN (1- | PRT | MGKHHHHSGHHHTGHHHHSGSHHHTSSSASTGENLYFQGTGDGSGE |
| | 144) | | NGESVVVLDKYGYPILYAPEEIGEEKEYSKYEDVVIEWNPSVTPVQ |
| | construct | | IEKNYEVKFDVRQVKLRPPKVKNGSGKEGEIIVEAYASLFKSRLSK |
| | | | LKRILRENPEISNVVDIGKLNYVSGDEEVTIIGLVNSKRETNRGLI |
| | | | FEVEDKTGIVKVFLPKDSEDYREAFKVLPDAVVAFKGFYSKKGIFF |
| | | | ANKFYLPDVPLYRKQKPPLEEKVYAILISDIHVGSREFCEKAFLKF |
| | | | LEWLNGHVESKEEEEIVSRVKYLIIAGDVVDGIGIYPGQYSDLVIP |
| | | | DIFDQYEALANLLANVPEHITMFIGPGNHDAARPAIPQPEFYKEYA |
| | | | KPIYKLKNAIIISNPAVIRLHGRDFLIAHGRGIEDVVSFVPGLTHH |
| | | | KPGLPMVELLKMRHLAPTFGGKVPIAPDPEDLLVIEEVPDLVQMGH |
| | | | VHVYDAVVYRGVQLVNSATWQAQTEFQKMVNIVPTPAKVPVVDVES |
| | | | ARVVKVLDFSGWC |
| 19 | DP1-ΔN (1- | PRT | MGKHHHHSGHHHTGHHHHSGSHHHTSSSASTGENLYFQGTGDGSGE |
| | 144)-H451A | | NGESVVVLDKYGYPILYAPEEIGEEKEYSKYEDVVIEWNPSVTPVQ |
| | construct | | IEKNYEVKFDVRQVKLRPPKVKNGSGKEGEIIVEAYASLFKSRLSK |
| | | | LKRILRENPEISNVVDIGKLNYVSGDEEVTIIGLVNSKRETNRGLI |
| | | | FEVEDKTGIVKVFLPKDSEDYREAFKVLPDAVVAFKGFYSKKGIFF |
| | | | ANKFYLPDVPLYRKQKPPLEEKVYAILISDIHVGSREFCEKAFLKF |
| | | | LEWLNGHVESKEEEEIVSRVKYLIIAGDVVDGIGIYPGQYSDLVIP |
| | | | DIFDQYEALANLLANVPEHITMFIGPGNADAARPAIPQPEFYKEYA |
| | | | KPIYKLKNAIIISNPAVIRLHGRDFLIAHGRGIEDVVSFVPGLTHH |
| | | | KPGLPMVELLKMRHLAPTFGGKVPIAPDPEDLLVIEEVPDLVQMGH |
| | | | VHVYDAVVYRGVQLVNSATWQAQTEFQKMVNIVPTPAKVPVVDVES |
| | | | ARVVKVLDFSGWC |
| 20 | DP1 wt | DNA | ccatgggcaagcatcaccatcattcaggccatcaccataccggaca |
| | construct | | ccaccatcattcaggcagtcatcaccatactagtagcagcgctagc |
| | | | accggcgagaacctgtattttcaagggacgggggatggatcCGATG |
| | | | AACTGGTTAAAGCACTGGAACGTGCAGGTTATCTGCTGACCCCGAG |
| | | | CGCATATTACCTGCTGGTTGATCATTTTAAAGAGGGCAAATTTTCA |
| | | | CTGGTGGAACTGGTGAAATTTGCCAAAAGCAAAGGCGTGTTTATTA |
| | | | TCGATGGTGATCTGGCCTATGAATTCCTGCAGTTTTTAGGTCTGGG |
| | | | TGTTCCGCAAGAAATCAAAGAAAGCTATATTAGCACCGGTGAAGAA |
| | | | GCAGAGAAAACCGTTGAAAGCCAAGAAACCCGTGCAAGCGAACTGG |
| | | | AAGAAGGTGGCGTTAGCCAGGTGAGCAGCGGTGAACTGCAAGAACT |
| | | | GAAAGAAGAAAGTCCGGAAATTAGCACCACCGAAGAAGAAATTGGC |
| | | | GGTCTGGAATTAGTTCAGAGCAGCATTAGTACCGGTAGCGAAGTTG |
| | | | AATACAATAATGGTGAAAATGGCGAAAGCGTTGTGGTGCTGGATAA |
| | | | ATATGGTTATCCGATTCTGTATGCACCGGAAGAAATCGGCGAAGAA |
| | | | AAAGAGTATAGCAAATACGAAGATGTGGTGATTGAATGGAATCCGA |
| | | | GCGTTACACCGGTTCAGATTGAAAAGAATTACGAGGTGAAATTCGA |
| | | | TGTGCGTCAGGTTAAACTGCGTCCGCCTAAAGTTAAAAATGGTAGC |
| | | | GGTAAAGAAGGCGAGATTATCGTTGAAGCCTATGCAAGCCTGTTTA |
| | | | AAAGCCGTCTGAGCAAACTGAAACGTATTCTGCGTGAAAACCCGGA |
| | | | AATTTCAAATGTGGTGGATATTGGCAAGCTGAATTATGTTAGCGGT |
| | | | GATGAAGAAGTGACGATTATTGGTCTGGTTAATAGCAAACGCGAAA |
| | | | CCAATCGTGGTCTGATTTTTGAGGTTGAAGATAAAACCGGCATCGT |
| | | | GAAAGTTTTTCTGCCGAAAGATAGCGAAGATTATCGCGAAGCATTT |
| | | | AAAGTTCTGCCGGATGCAGTTGTTGCGTTTAAAGGTTTTTATAGCA |
| | | | AGAAAGGCATCTTCTTCGCCAACAAATTTTACCTGCCTGATGTTCC |
| | | | GCTGTATCGTAAACAGAAACCGCCTCTGGAAGAGAAAGTTTATGCA |
| | | | ATTCTGATCAGCGATATTCATGTTGGCAGCCGTGAATTTTGTGAAA |
| | | | AAGCCTTTCTGAAATTCCTGGAATGGCTGAATGGTCATGTGGAATC |
| | | | TAAAGAAGAAGAGGAAATTGTCAGTCGCGTCAAGTATCTGATTATT |
| | | | GCCGGTGATGTTGTTGATGGCATTGGTATTTATCCGGGTCAGTATA |
| | | | GCGATCTGGTTATCCCGGATATTTTTGATCAGTATGAAGCACTGGC |
| | | | AAATCTGCTGGCAAATGTGCCGGAACATATCACCATGTTTATTGGT |
| | | | CCGGGTAATCATGATGCAGCACGTCCGGCAATTCCGCAGCCGGAAT |
| | | | TCTATAAAGAATATGCCAAACCGATCTATAAACTGAAAAACGCCAT |
| | | | CATCATTAGCAATCCGGCAGTTATTCGTCTGCATGGTCGTGATTTT |
| | | | CTGATTGCACATGGTCGCGGTATTGAAGATGTTGTTAGCTTTGTTC |
| | | | CGGGTCTGACCCATCATAAACCGGGTCTGCCGATGGTTGAACTGCT |
| | | | GAAAATGCGTCATCTGGCACCGACCTTTGGTGGTAAAGTTCCGATT |
| | | | GCACCTGATCCGGAAGATCTGCTGGTTATTGAAGAAGTTCCGGATC |
| | | | TGGTTCAGATGGGTCATGTTCATGTGTATGATGCCGTTGTTTATCG |
| | | | TGGTGTTCAGCTGGTGAATAGCGCAACCTGGCAGGCACAGACCGAA |
| | | | TTCCAGAAAATGGTTAATATTGTTCCGACACCGGCAAAAGTTCCGG |
| | | | TTGTGGATGTTGAAAGCGCACGTGTTGTTAAAGTGCTGGATTTTAG |
| | | | CGGTTGGTGCTAAgcggccgc |
| 21 | DP2 wt | DNA | catatggagcttccaaaggaaatggaagagtatttcgaaatgctgc |
| | construct | | agcgtgagattgataaggcctatgagattgctaaaaaggcgcgtgc |
| | | | tcagggtaaagatccgagcttggatgttgagattcctcaagctacc |
| | | | gatatggccggacgtgttgaaagcttagttggcccgccaggagttg |
| | | | ctaaacgtattcgtgagttagttaaagagtatggaaaggagattgc |
| | | | cgccctcaaaattgttgacgagattattgaaggtaaattcggcgac |
| | | | cttggaagtcgtgagaagtacgctgagcaggccgtgcgtactgcat |
| | | | tggcaattctgaccgagggaattgtttccgctccgattgagggtat |
| | | | tgctaacgtcaagattaagcgtaacacttgggctgacaactccgag |
| | | | tatctggccctctactatgcgggcccaattcgtagctcaggtggaa |
| | | | ccgctcaggcccttagcgttttggttggtgattacgttcgtcgtaa |
| | | | gctcggcctggaccgttttaagccaagcgagaagcacattgagcgt |
| | | | atggttgaagaggtggatctttatcatcgtgccgtcactcgtttgc |
| | | | agtatcatccgtctccagaggaagttcgtcttgctatgcgtaacat |
| | | | tccaatcgagattacaggtgaggcaacggatgatgttgaagtctcc |
| | | | catcgtgacgttcctggggtcgaaactaatcagcttcgtggcggtg |
| | | | caattctggttctggctgaaggtgtcctccaaaaggctaagaagct |
| | | | tgttaaatatattgacaagatggggattgagggttgggaatggctc |
| | | | aaggaattcgttgaggctaaggagaagggagaacctaaggaggaag |
| | | | gtaaagaggaaagcctggctgaatcaacccttgaggaaacgaaggt |
| | | | cgaagttgatatgggcttctactactctctttatcagaagttcaag |
| | | | gaggatatcgctccaagcgacaagtacgctaaggaagttattgggg |
| | | | gccgtcctttattctctgatccatctaaaccaggtggatttcgttt |
| | | | gcgttacgggcgtagtcgtgcgagcgggttcgcaacttggggaatt |
| | | | aaccctgcaaccatgattttagttgacgagttcttggcaattggaa |
| | | | ctcaactgaaaaccgaacgtccagggaagggtgcggtggttactcc |
| | | | agtaacgacgattgagggcccaattgtcaagctcaaggatggtagc |
| | | | gttctgcgtgttgatgattataatttggccctcaaggttcgtgaag |
| | | | atgttgaggagattctttaccttggtgacgcggtgattgctttcgg |
| | | | agatttcgttgagaacaaccaaaccctccttccagctaattattgc |
| | | | gaggagtggtggattcttgagttcgtcaaggccctcaaagagattt |
| | | | atgaggtccaccttgagccttttaccgaaaatgaggaagagagcat |
| | | | tgaggaagcttctgattacctggagattgacccagaatttttaaag |
| | | | gaaatgctccgtgatccgttgcgtgtaaaacctccggttgagcttg |
| | | | ccattcacttctccgaggttcttggaattccgttgcatccgtatta |
| | | | caccttgtactggaattccgtcgagccgaaagacgttgaaaagctg |
| | | | tggcgtttgctcaaaaattatgccgagattgaatggagcaactttc |
| | | | gtggaattaaatttgcgaagaaaattgtcatttcccaagaaaagct |
| | | | tggagattctaagcgtactttagagctcctgggattgcctcacacc |
| | | | gttcgtgatgggaacgttattgtggactatccgtgggctgccgccc |
| | | | tcttaactccgctcggcaacttgaattgggagttcatggcaaagcc |
| | | | gctttacgccaccattgatattatcaacgagaataacgagattaag |
| | | | cttcgtgatcgtggaattagctggattggcgcgcgtatgggtcgtc |
| | | | cagagaaggcgaaagagcgtaagatgaagcctcctgttcaagttct |
| | | | gttcccaattggtctggccggcgggagcagtcgtgacattaagaaa |
| | | | gccgctgaagagggtaaagtggccgaggttgagattgctttcttca |
| | | | agtgcccgaagtgcggtcacgttggcccagagcatttatgtccaaa |
| | | | ctgtggaacgcgtaaagaacttctctgggtctgtccacgttgtaac |
| | | | gcggagtaccctgagagccaggccgagggctataattacacttgtc |
| | | | caaagtgcaacgtcaagctgcgtccgtacgccaagcgtaagattcg |
| | | | tccttccgaattgctgaatcgtgcgatggagaacgtcaaagtctac |
| | | | ggagttgacaagttaaagggagtcatgggaatgacttctggctgga |
| | | | agatgccggagccacttgagaaaggtcttctgcgtgctaaaaacga |
| | | | tgtttacgtgttcaaggatggtaccattcgttttgacgcaacagat |
| | | | gccccaattacccacttccgtccgcgtgaaattggagtttccgttg |
| | | | agaagttacgtgaacttggttatactcatgacttcgagggtaaacc |
| | | | gctggttagcgaggatcaaattgttgagctcaagccgcaggatatt |
| | | | atcctgtcaaaggaagccgggcgttatctcctgaaggtggccaagt |
| | | | ttgttgatgacctccttgagaaattctacggcttgccgcgttttta |
| | | | caacgccgaaaagatggaagatttaattggccatctcgtcattggc |
| | | | ttggctccacacacctccgctggtattgtgggtcgtattattggct |
| | | | ttgtcgatgccctggttggttacgctcacccgtacttccacgctgc |
| | | | gaagcgtcgtaactgtgatggagatgaagacgctgttatgctcctc |
| | | | ctggatgccctcctgaacttctctcgctattaccttccggaaaagc |
| | | | gtggtggaaagatggacgctcctttggtaattacaacgcgtcttga |
| | | | tccgcgtgaagttgatagcgaagtccataacatggacattgtccgt |
| | | | tattatccgctggagttctacgaggcaacttatgagcttaaatccc |
| | | | cgaaggaactcgttggggtaattgaacgtgttgaggatcgcctggg |
| | | | aaagcctgagatgtactatggcctgaagttcacccacgatacggat |
| | | | gacattgctttgggtccaaagatgagtctgtacaagcagttagggg |
| | | | acatggaagagaaggtccgtcgtcagcttgaagttgctaagcgtat |
| | | | tcgtgctgtggatgaacatggagttgccgagaagattttgaattcc |
| | | | cacctgattcctgacttacgtggtaatctgcgtagctttacgcgtc |
| | | | aagagttccgttgcgtcaagtgcaacactaagttccgtcgtccgcc |
| | | | actgaatggaaagtgcccggtttgcggaggtaaaattgtccttacc |
| | | | gtgagcaagggggcaatcgagaaatacttgggaactgccaagatgc |
| | | | tcgttaccgaatacaacgtgaagaattacacgcgtcagcgtatttg |
| | | | cttgactgaacgtgacattgattcactgttcgaaaacgtgttccca |
| | | | gagacccagttaactttaatcgttaatccaaatgatatttgccagc |
| | | | gtctcgtgatggcgcgtactggagaagttaataagtcaggcctcct |
| | | | tgaaaatctttctaacggttcgaagaagactgagaaagctgaaaag |
| | | | gccgagaagccgcgtaagaaatcagatgagaagccaaagaagaaac |
| | | | gtgttatcagcctcgaagagttcttttcacgtaaatcaaagtagct |
| | | | cgag |
| 22 | DP1-H451A | DNA | ccatgggcaagcatcaccatcattcaggccatcaccataccggaca |
| | construct | | ccaccatcattcaggcagtcatcaccatactagtagcagcgctagc |
| | | | accggcgagaacctgtattttcaagggacgggggatggatcCGATG |
| | | | AACTGGTTAAAGCACTGGAACGTGCAGGTTATCTGCTGACCCCGAG |
| | | | CGCATATTACCTGCTGGTTGATCATTTTAAAGAGGGCAAATTTTCA |
| | | | CTGGTGGAACTGGTGAAATTTGCCAAAAGCAAAGGCGTGTTTATTA |
| | | | TCGATGGTGATCTGGCCTATGAATTCCTGCAGTTTTTAGGTCTGGG |
| | | | TGTTCCGCAAGAAATCAAAGAAAGCTATATTAGCACCGGTGAAGAA |
| | | | GCAGAGAAAACCGTTGAAAGCCAAGAAACCCGTGCAAGCGAACTGG |
| | | | AAGAAGGTGGCGTTAGCCAGGTGAGCAGCGGTGAACTGCAAGAACT |
| | | | GAAAGAAGAAAGTCCGGAAATTAGCACCACCGAAGAAGAAATTGGC |
| | | | GGTCTGGAATTAGTTCAGAGCAGCATTAGTACCGGTAGCGAAGTTG |
| | | | AATACAATAATGGTGAAAATGGCGAAAGCGTTGTGGTGCTGGATAA |
| | | | ATATGGTTATCCGATTCTGTATGCACCGGAAGAAATCGGCGAAGAA |
| | | | AAAGAGTATAGCAAATACGAAGATGTGGTGATTGAATGGAATCCGA |
| | | | GCGTTACACCGGTTCAGATTGAAAAGAATTACGAGGTGAAATTCGA |
| | | | TGTGCGTCAGGTTAAACTGCGTCCGCCTAAAGTTAAAAATGGTAGC |
| | | | GGTAAAGAAGGCGAGATTATCGTTGAAGCCTATGCAAGCCTGTTTA |
| | | | AAAGCCGTCTGAGCAAACTGAAACGTATTCTGCGTGAAAACCCGGA |
| | | | AATTTCAAATGTGGTGGATATTGGCAAGCTGAATTATGTTAGCGGT |
| | | | GATGAAGAAGTGACGATTATTGGTCTGGTTAATAGCAAACGCGAAA |
| | | | CCAATCGTGGTCTGATTTTTGAGGTTGAAGATAAAACCGGCATCGT |
| | | | GAAAGTTTTTCTGCCGAAAGATAGCGAAGATTATCGCGAAGCATTT |
| | | | AAAGTTCTGCCGGATGCAGTTGTTGCGTTTAAAGGTTTTTATAGCA |
| | | | AGAAAGGCATCTTCTTCGCCAACAAATTTTACCTGCCTGATGTTCC |
| | | | GCTGTATCGTAAACAGAAACCGCCTCTGGAAGAGAAAGTTTATGCA |
| | | | ATTCTGATCAGCGATATTCATGTTGGCAGCCGTGAATTTTGTGAAA |
| | | | AAGCCTTTCTGAAATTCCTGGAATGGCTGAATGGTCATGTGGAATC |
| | | | TAAAGAAGAAGAGGAAATTGTCAGTCGCGTCAAGTATCTGATTATT |
| | | | GCCGGTGATGTTGTTGATGGCATTGGTATTTATCCGGGTCAGTATA |
| | | | GCGATCTGGTTATCCCGGATATTTTTGATCAGTATGAAGCACTGGC |
| | | | AAATCTGCTGGCAAATGTGCCGGAACATATCACCATGTTTATTGGT |
| | | | CCGGGTAATGCGGATGCAGCACGTCCGGCAATTCCGCAGCCGGAAT |
| | | | TCTATAAAGAATATGCCAAACCGATCTATAAACTGAAAAACGCCAT |
| | | | CATCATTAGCAATCCGGCAGTTATTCGTCTGCATGGTCGTGATTTT |
| | | | CTGATTGCACATGGTCGCGGTATTGAAGATGTTGTTAGCTTTGTTC |
| | | | CGGGTCTGACCCATCATAAACCGGGTCTGCCGATGGTTGAACTGCT |
| | | | GAAAATGCGTCATCTGGCACCGACCTTTGGTGGTAAAGTTCCGATT |
| | | | GCACCTGATCCGGAAGATCTGCTGGTTATTGAAGAAGTTCCGGATC |
| | | | TGGTTCAGATGGGTCATGTTCATGTGTATGATGCCGTTGTTTATCG |
| | | | TGGTGTTCAGCTGGTGAATAGCGCAACCTGGCAGGCACAGACCGAA |
| | | | TTCCAGAAAATGGTTAATATTGTTCCGACACCGGCAAAAGTTCCGG |
| | | | TTGTGGATGTTGAAAGCGCACGTGTTGTTAAAGTGCTGGATTTTAG |
| | | | CGGTTGGTGCTAAgcggccgc |
| 23 | DP1-ΔN (1- | DNA | ccATGGGCAAGCATCACCATCATTCAGGCCATCACCATACCGGACA |
| | 144) | | CCACCATCATTCAGGCAGTCATCACCATACTAGTAGCAGCGCTAGC |
| | construct | | ACCGGCGAGAACCTGTATTTTCAAGGGACGGGGGATGGATCCGGTG |
| | | | AAAATGGCGAAAGCGTTGTGGTGCTGGATAAATATGGTTATCCGAT |
| | | | TCTGTATGCACCGGAAGAAATCGGCGAAGAAAAAGAGTATAGCAAA |
| | | | TACGAAGATGTGGTGATTGAATGGAATCCGAGCGTTACACCGGTTC |
| | | | AGATTGAAAAGAATTACGAGGTGAAATTCGATGTGCGTCAGGTTAA |
| | | | ACTGCGTCCGCCTAAAGTTAAAAATGGTAGCGGTAAAGAAGGCGAG |
| | | | ATTATCGTTGAAGCCTATGCAAGCCTGTTTAAAAGCCGTCTGAGCA |
| | | | AACTGAAACGTATTCTGCGTGAAAACCCGGAAATTTCAAATGTGGT |
| | | | GGATATTGGCAAGCTGAATTATGTTAGCGGTGATGAAGAAGTGACG |
| | | | ATTATTGGTCTGGTTAATAGCAAACGCGAAACCAATCGTGGTCTGA |
| | | | TTTTTGAGGTTGAAGATAAAACCGGCATCGTGAAAGTTTTTCTGCC |
| | | | GAAAGATAGCGAAGATTATCGCGAAGCATTTAAAGTTCTGCCGGAT |
| | | | GCAGTTGTTGCGTTTAAAGGTTTTTATAGCAAGAAAGGCATCTTCT |
| | | | TCGCCAACAAATTTTACCTGCCTGATGTTCCGCTGTATCGTAAACA |
| | | | GAAACCGCCTCTGGAAGAGAAAGTTTATGCAATTCTGATCAGCGAT |
| | | | ATTCATGTTGGCAGCCGTGAATTTTGTGAAAAAGCCTTTCTGAAAT |
| | | | TCCTGGAATGGCTGAATGGTCATGTGGAATCTAAAGAAGAAGAGGA |
| | | | AATTGTCAGTCGCGTCAAGTATCTGATTATTGCCGGTGATGTTGTT |
| | | | GATGGCATTGGTATTTATCCGGGTCAGTATAGCGATCTGGTTATCC |
| | | | CGGATATTTTTGATCAGTATGAAGCACTGGCAAATCTGCTGGCAAA |
| | | | TGTGCCGGAACATATCACCATGTTTATTGGTCCGGGTAATCATGAT |
| | | | GCAGCACGTCCGGCAATTCCGCAGCCGGAATTCTATAAAGAATATG |
| | | | CCAAACCGATCTATAAACTGAAAAACGCCATCATCATTAGCAATCC |
| | | | GGCAGTTATTCGTCTGCATGGTCGTGATTTTCTGATTGCACATGGT |
| | | | CGCGGTATTGAAGATGTTGTTAGCTTTGTTCCGGGTCTGACCCATC |
| | | | ATAAACCGGGTCTGCCGATGGTTGAACTGCTGAAAATGCGTCATCT |
| | | | GGCACCGACCTTTGGTGGTAAAGTTCCGATTGCACCTGATCCGGAA |
| | | | GATCTGCTGGTTATTGAAGAAGTTCCGGATCTGGTTCAGATGGGTC |
| | | | ATGTTCATGTGTATGATGCCGTTGTTTATCGTGGTGTTCAGCTGGT |
| | | | GAATAGCGCAACCTGGCAGGCACAGACCGAATTCCAGAAAATGGTT |
| | | | AATATTGTTCCGACACCGGCAAAAGTTCCGGTTGTGGATGTTGAAA |
| | | | GCGCACGTGTTGTTAAAGTGCTGGATTTTAGCGGTTGGTGCTAAgc |
| | | | ggccgc |
| 24 | DP1-ΔN (1- | nt | ccatgggcaagcatcaccatcattcaggccatcaccataccggaca |
| | 144)-H451A | | ccaccatcattcaggcagtcatcaccatactagtagcagcgctagc |
| | construct | | accggcgagaacctgtattttcaagggacgggggatggatcCGGTG |
| | | | AAAATGGCGAAAGCGTTGTGGTGCTGGATAAATATGGTTATCCGAT |
| | | | TCTGTATGCACCGGAAGAAATCGGCGAAGAAAAAGAGTATAGCAAA |
| | | | TACGAAGATGTGGTGATTGAATGGAATCCGAGCGTTACACCGGTTC |
| | | | AGATTGAAAAGAATTACGAGGTGAAATTCGATGTGCGTCAGGTTAA |
| | | | ACTGCGTCCGCCTAAAGTTAAAAATGGTAGCGGTAAAGAAGGCGAG |
| | | | ATTATCGTTGAAGCCTATGCAAGCCTGTTTAAAAGCCGTCTGAGCA |
| | | | AACTGAAACGTATTCTGCGTGAAAACCCGGAAATTTCAAATGTGGT |
| | | | GGATATTGGCAAGCTGAATTATGTTAGCGGTGATGAAGAAGTGACG |
| | | | ATTATTGGTCTGGTTAATAGCAAACGCGAAACCAATCGTGGTCTGA |
| | | | TTTTTGAGGTTGAAGATAAAACCGGCATCGTGAAAGTTTTTCTGCC |
| | | | GAAAGATAGCGAAGATTATCGCGAAGCATTTAAAGTTCTGCCGGAT |
| | | | GCAGTTGTTGCGTTTAAAGGTTTTTATAGCAAGAAAGGCATCTTCT |
| | | | TCGCCAACAAATTTTACCTGCCTGATGTTCCGCTGTATCGTAAACA |
| | | | GAAACCGCCTCTGGAAGAGAAAGTTTATGCAATTCTGATCAGCGAT |
| | | | ATTCATGTTGGCAGCCGTGAATTTTGTGAAAAAGCCTTTCTGAAAT |
| | | | TCCTGGAATGGCTGAATGGTCATGTGGAATCTAAAGAAGAAGAGGA |
| | | | AATTGTCAGTCGCGTCAAGTATCTGATTATTGCCGGTGATGTTGTT |
| | | | GATGGCATTGGTATTTATCCGGGTCAGTATAGCGATCTGGTTATCC |
| | | | CGGATATTTTTGATCAGTATGAAGCACTGGCAAATCTGCTGGCAAA |
| | | | TGTGCCGGAACATATCACCATGTTTATTGGTCCGGGTAATGCGGAT |
| | | | GCAGCACGTCCGGCAATTCCGCAGCCGGAATTCTATAAAGAATATG |
| | | | CCAAACCGATCTATAAACTGAAAAACGCCATCATCATTAGCAATCC |
| | | | GGCAGTTATTCGTCTGCATGGTCGTGATTTTCTGATTGCACATGGT |
| | | | CGCGGTATTGAAGATGTTGTTAGCTTTGTTCCGGGTCTGACCCATC |
| | | | ATAAACCGGGTCTGCCGATGGTTGAACTGCTGAAAATGCGTCATCT |
| | | | GGCACCGACCTTTGGTGGTAAAGTTCCGATTGCACCTGATCCGGAA |
| | | | GATCTGCTGGTTATTGAAGAAGTTCCGGATCTGGTTCAGATGGGTC |
| | | | ATGTTCATGTGTATGATGCCGTTGTTTATCGTGGTGTTCAGCTGGT |
| | | | GAATAGCGCAACCTGGCAGGCACAGACCGAATTCCAGAAAATGGTT |
| | | | AATATTGTTCCGACACCGGCAAAAGTTCCGGTTGTGGATGTTGAAA |
| | | | GCGCACGTGTTGTTAAAGTGCTGGATTTTAGCGGTTGGTGCTAAgc |
| | | | ggccgc |
| 25 | DP2-ΔC (1217- | nt | catatggagcttccaaaggaaatggaagagtatttcgaaatgctgc |
| | 1270) | | agcgtgagattgataaggcctatgagattgctaaaaaggcgcgtgc |
| | construct | | tcagggtaaagatccgagcttggatgttgagattcctcaagctacc |
| | | | gatatggccggacgtgttgaaagcttagttggcccgccaggagttg |
| | | | ctaaacgtattcgtgagttagttaaagagtatggaaaggagattgc |
| | | | cgccctcaaaattgttgacgagattattgaaggtaaattcggcgac |
| | | | cttggaagtcgtgagaagtacgctgagcaggccgtgcgtactgcat |
| | | | tggcaattctgaccgagggaattgtttccgctccgattgagggtat |
| | | | tgctaacgtcaagattaagcgtaacacttgggctgacaactccgag |
| | | | tatctggccctctactatgcgggcccaattcgtagctcaggtggaa |
| | | | ccgctcaggcccttagcgttttggttggtgattacgttcgtcgtaa |
| | | | gctcggcctggaccgttttaagccaagcgagaagcacattgagcgt |
| | | | atggttgaagaggtggatctttatcatcgtgccgtcactcgtttgc |
| | | | agtatcatccgtctccagaggaagttcgtcttgctatgcgtaacat |
| | | | tccaatcgagattacaggtgaggcaacggatgatgttgaagtctcc |
| | | | catcgtgacgttcctggggtcgaaactaatcagcttcgtggcggtg |
| | | | caattctggttctggctgaaggtgtcctccaaaaggctaagaagct |
| | | | tgttaaatatattgacaagatggggattgagggttgggaatggctc |
| | | | aaggaattcgttgaggctaaggagaagggagaacctaaggaggaag |
| | | | gtaaagaggaaagcctggctgaatcaacccttgaggaaacgaaggt |
| | | | cgaagttgatatgggcttctactactctctttatcagaagttcaag |
| | | | gaggatatcgctccaagcgacaagtacgctaaggaagttattgggg |
| | | | gccgtcctttattctctgatccatctaaaccaggtggatttcgttt |
| | | | gcgttacgggcgtagtcgtgcgagcgggttcgcaacttggggaatt |
| | | | aaccctgcaaccatgattttagttgacgagttcttggcaattggaa |
| | | | ctcaactgaaaaccgaacgtccagggaagggtgcggtggttactcc |
| | | | agtaacgacgattgagggcccaattgtcaagctcaaggatggtagc |
| | | | gttctgcgtgttgatgattataatttggccctcaaggttcgtgaag |
| | | | atgttgaggagattctttaccttggtgacgcggtgattgctttcgg |
| | | | agatttcgttgagaacaaccaaaccctccttccagctaattattgc |
| | | | gaggagtggtggattcttgagttcgtcaaggccctcaaagagattt |
| | | | atgaggtccaccttgagccttttaccgaaaatgaggaagagagcat |
| | | | tgaggaagcttctgattacctggagattgacccagaatttttaaag |
| | | | gaaatgctccgtgatccgttgcgtgtaaaacctccggttgagcttg |
| | | | ccattcacttctccgaggttcttggaattccgttgcatccgtatta |
| | | | caccttgtactggaattccgtcgagccgaaagacgttgaaaagctg |
| | | | tggcgtttgctcaaaaattatgccgagattgaatggagcaactttc |
| | | | gtggaattaaatttgcgaagaaaattgtcatttcccaagaaaagct |
| | | | tggagattctaagcgtactttagagctcctgggattgcctcacacc |
| | | | gttcgtgatgggaacgttattgtggactatccgtgggctgccgccc |
| | | | tcttaactccgctcggcaacttgaattgggagttcatggcaaagcc |
| | | | gctttacgccaccattgatattatcaacgagaataacgagattaag |
| | | | cttcgtgatcgtggaattagctggattggcgcgcgtatgggtcgtc |
| | | | cagagaaggcgaaagagcgtaagatgaagcctcctgttcaagttct |
| | | | gttcccaattggtctggccggcgggagcagtcgtgacattaagaaa |
| | | | gccgctgaagagggtaaagtggccgaggttgagattgctttcttca |
| | | | agtgcccgaagtgcggtcacgttggcccagagcatttatgtccaaa |
| | | | ctgtggaacgcgtaaagaacttctctgggtctgtccacgttgtaac |
| | | | gcggagtaccctgagagccaggccgagggctataattacacttgtc |
| | | | caaagtgcaacgtcaagctgcgtccgtacgccaagcgtaagattcg |
| | | | tccttccgaattgctgaatcgtgcgatggagaacgtcaaagtctac |
| | | | ggagttgacaagttaaagggagtcatgggaatgacttctggctgga |
| | | | agatgccggagccacttgagaaaggtcttctgcgtgctaaaaacga |
| | | | tgtttacgtgttcaaggatggtaccattcgttttgacgcaacagat |
| | | | gccccaattacccacttccgtccgcgtgaaattggagtttccgttg |
| | | | agaagttacgtgaacttggttatactcatgacttcgagggtaaacc |
| | | | gctggttagcgaggatcaaattgttgagctcaagccgcaggatatt |
| | | | atcctgtcaaaggaagccgggcgttatctcctgaaggtggccaagt |
| | | | ttgttgatgacctccttgagaaattctacggcttgccgcgttttta |
| | | | caacgccgaaaagatggaagatttaattggccatctcgtcattggc |
| | | | ttggctccacacacctccgctggtattgtgggtcgtattattggct |
| | | | ttgtcgatgccctggttggttacgctcacccgtacttccacgctgc |
| | | | gaagcgtcgtaactgtgatggagatgaagacgctgttatgctcctc |
| | | | ctggatgccctcctgaacttctctcgctattaccttccggaaaagc |
| | | | gtggtggaaagatggacgctcctttggtaattacaacgcgtcttga |
| | | | tccgcgtgaagttgatagcgaagtccataacatggacattgtccgt |
| | | | tattatccgctggagttctacgaggcaacttatgagcttaaatccc |
| | | | cgaaggaactcgttggggtaattgaacgtgttgaggatcgcctggg |
| | | | aaagcctgagatgtactatggcctgaagttcacccacgatacggat |
| | | | gacattgctttgggtccaaagatgagtctgtacaagcagttagggg |
| | | | acatggaagagaaggtccgtcgtcagcttgaagttgctaagcgtat |
| | | | tcgtgctgtggatgaacatggagttgccgagaagattttgaattcc |
| | | | cacctgattcctgacttacgtggtaatctgcgtagctttacgcgtc |
| | | | aagagttccgttgcgtcaagtgcaacactaagttccgtcgtccgcc |
| | | | actgaatggaaagtgcccggtttgcggaggtaaaattgtccttacc |
| | | | gtgagcaagggggcaatcgagaaatacttgggaactgccaagatgc |
| | | | tcgttaccgaatacaacgtgaagaattacacgcgtcagcgtatttg |
| | | | cttgactgaacgtgacattgattcactgttcgaaaacgtgttccca |
| | | | gagacccagttaactttaatcgttaatccaaatgatatttgccagc |
| | | | gtctcgtgatggcgcgttaactcgag |
| 26 | Tag | PRT | MGKHHHHSGHHHTGHHHHSGSHHHTSSSASTGENLYFQGTGDGS |
| 27 | TEV cleavage | PRT | ENLYFQG |
| | site | | |
| 28 | Primer | DNA | ctactactctctttatcagaagttcaaggaggat |
| | P2.6kb_Fwd | | |
| 29 | Primer | DNA | cgattaaagttaactgggtctctgggaa |
| | P2. 6kb_rev | | |
| 30 | Primer | DNA | 6FAM-GAGGTCTCGCTCCGACCGCTCCCG |
| 31 | Template | DNA | AGTGCCTAACGA-TG-CGGGAGCGGTCGGAGCGAGACCTC |
| | DNA12 | | |
| 32 | Template | RNA/ | (AGUGCCUAACGA) -TG-CGGGAGCGGTCGGAGCGAGACCTC |
| | RNA12 | DNA | |
| 33 | Template | RNA/ | [AGUGCCUAACGA] - TG-CGGGAGCGGTCGGAGCGAGACCTC |
| | 2′-O-MeRNA12 | DNA | |
| 34 | Template | DNA | AGTGCCTAACCAAGTGCCTAACCAAGTGCCTAACGA-TG- |
| | DNA36 | | CGGGAGCGGTCGGAGCGAGACCTC |
| 35 | Template | RNA/ | (AGUGCCUAACCAAGUGCCUAACCAAGUGCCUAACGA) -TG- |
| | RNA36 | DNA | CGGGAGCGGTCGGAGCGAGACCTC |

1-15. (canceled)

16. An engineered DNA polymerase of the family D (PolD) comprising:
(i) a truncated subunit DP1 comprising a N-terminal deletion of at least the helix-strand-helix (HSH) domain from positions 1 to 67, the indicated positions being determined by alignment with SEQ ID NO: 1; and
(ii) a truncated subunit DP2 comprising a C-terminal deletion of at least the residues from positions 1220 to 1270, the indicated positions being determined by alignment with SEQ ID NO: 2.

17. The engineered PolD according to claim 16, wherein the N-terminal deletion of DP1 is from position 1 to any one of positions 67 to 196, the indicated positions being determined by alignment with SEQ ID NO: 1.

18. The engineered PolD according to claim 17, wherein the N-terminal deletion of DP1 is from positions 1 to 144 or from positions 1 to 196, the indicated positions being determined by alignment with SEQ ID NO: 1.

19. The engineered PolD according to claim 16, wherein the C-terminal deletion of DP2 is from any one of positions 1191 to 1220 to position 1270, the indicated positions being determined by alignment with SEQ ID NO: 2.

20. The engineered PolD according to claim 19, wherein the C-terminal deletion of DP2 is from positions 1194 to 1270, 1195 to 1270 or 1217 to 1270, the indicated positions being determined by alignment with SEQ ID NO: 2.

21. The engineered PolD according to claim 16, wherein the truncated subunits are from DP1 and DP2 of a Thermococcales archaea chosen from Pyrococcus abyssi, Pyrococcus furiosus, Thermococcus nautili, Thermococcus kodakarensis, Thermococcus barophilus, Palaeococcus ferrophilus, and a functional variant thereof.

22. The engineered PolD according to claim 21, wherein the truncated subunits are from DP1 of any one of SEQ ID NO: 1 and 3 to 7 and DP2 of any one of SEQ ID NO: 2 and 8 to 12, or a functional variant thereof.

23. The engineered PolD according to claim 16, wherein the truncated DP1 subunit comprises a truncated DP1 amino acid sequence having at least 70% identity with the sequence from any one of positions 68 to 197 to position 619 of SEQ ID NO: 1.

24. The engineered PolD according to claim 23, wherein the truncated DP1 subunit comprises a truncated DP1 amino acid sequence having at least 70% identity with the sequence from positions 145 to 619 or 197 to 619 of SEQ ID NO: 1.

25. The engineered PolD according to claim 16, wherein the truncated DP2 subunit comprises a truncated DP2 amino acid sequence having at least 70% identity with the sequence from position 1 to any one of positions 1190 to 1219 of SEQ ID NO: 2.

26. The engineered PolD according to claim 25, wherein the truncated DP2 subunit comprises a truncated DP2 amino acid sequence having at least 70% identity with the sequence from positions 1 to 1193, 1 to 1194 or 1 to 1216 of SEQ ID NO: 2.

27. The engineered PolD according to claim 16, which is an exonuclease deficient variant comprising a DP1 subunit having at least one mutation which inactivates PolD exonuclease activity situated at any one of positions D360, H362, D404, Y412, N450, H451, H497, K536, H560, H562, F586 and V590, the indicated positions being determined by alignment with SEQ ID NO: 1.

28. The engineered PolD according to claim 27, wherein the exonuclease deficient variant is a DP1 variant chosen from H451A; D360A and H362A; or N450A, H560A and H562A.

29. An expression vector for the recombinant production of an engineered PolD according to claim 16 in a host cell, comprising a nucleic acid encoding said engineered PolD.

30. A method for amplifying a nucleic acid comprising incubating the engineered PolD according to claim 16 with a nucleic acid template, at least one oligonucleotide primer and nucleotides under conditions that allow amplification of the nucleic acid template.

31. A kit for nucleic acid amplification, in particular polymerase chain reaction (PCR), comprising at least an engineered PolD according to claim 16, and optionally, nucleotides, reaction buffer, and/or oligonucleotide primer(s).

32. A method for reverse transcription (RT) comprising incubating a polymerase of the family D (PolD) or a functional variant thereof with an RNA template, an oligonucleotide primer and nucleotides under conditions that allow reverse transcription of the RNA template, thereby obtaining a cDNA; optionally wherein the method is a method for reverse transcription (RT) and polymerase chain reaction (PCR), further comprising amplifying the obtained cDNA by PCR using said PolD or functional variant thereof.

33. The method according to claim 32, wherein the PolD is a thermostable PolD of a hyperthermophilic Thermococcales archaea chosen from Pyrococcus abyssi, Pyrococcus furiosus, Thermococcus nautili, Thermococcus kodakarensis, Thermococcus barophilus, Palaeococcus ferrophilus, a variant thereof, or an engineered PolD according to claim 1.

34. The method of claim 33, wherein the PolD is an exonuclease deficient PolD comprising a DP1 subunit having at least one mutation which inactivates PolD exonuclease activity situated at any one of positions D360, H362, D404, Y412, N450, H451, H497, K536, H560, H562, F586 and V590, the indicated positions being determined by alignment with SEQ ID NO: 1.

35. A kit for reverse transcription (RT) or for reverse transcription and polymerase chain reaction (RT-PCR) comprising a polymerase of the family D (PolD) or a functional variant thereof as defined in claim 16, wherein the kit does not comprise a reverse transcriptase.
